(19) United States
(12) Patent Application Publication
(10) Pub. No.: US 2023/0272037 A1
(43) Pub. Date: Aug. 31, 2023

(54) INHIBITORY CHIMERIC RECEPTOR ARCHITECTURES

(71) Applicant: Senti Biosciences, Inc., South San Francisco, CA (US)

(72) Inventors: Russell Morrison Gordley, San Francisco, CA (US); Marcela Guzman Ayala, San Francisco, CA (US); Gary Lee, Castro Valley, CA (US); Nicholas Frankel, San Francisco, CA (US)

(21) Appl. No.: 17/820,804

(22) Filed: Aug. 18, 2022

Related U.S. Application Data
(63) Continuation of application No. PCT/US2021/018847, filed on Feb. 19, 2021.
(60) Provisional application No. 63/127,843, filed on Dec. 18, 2020.
(60) Provisional application No. 62/979,310, filed on Feb. 20, 2020.

(51) Int. Cl. C07K 14/725; C07K 14/705; C07K 14/47; C12N 15/63

(52) U.S. Cl. CPC C07K 14/7051; C07K 14/70596; C07K 14/4703; C07K 14/70521; C12N 15/63; C07K 2319/03; C07K 2319/02; C07K 2317/622; C07K 2317/55

(57) ABSTRACT

Provided herein are inhibitory chimeric antigen receptor compositions and cells comprising such compositions. Also provided are methods of using inhibitory chimeric antigen receptors and cells.

### CROSS REFERENCE TO RELATED APPLICATIONS

This application is continuation of International Application No. PCT/US2021/018847, filed Feb. 19, 2021, which claims the benefit of U.S. Provisional Application Nos. 63/127,843 filed Dec. 18, 2020 and 62/979,310 filed Feb. 20, 2020, each of which is hereby incorporated by reference in their entirety for all purposes.

### SEQUENCE LISTING

The instant application contains a Sequence Listing which has been submitted electronically in XML format and is hereby incorporated by reference in its entirety. Said Sequence Listing XML, created on Sep. 1, 2022, is named STB-021WOC1_SL.xml, and is 126,885 bytes in size.

### BACKGROUND

Chimeric antigen receptors (CARs) enable targeted in vivo activation of immunomodulatory cells, such as T cells. These recombinant membrane receptors have an antigen-binding domain and one or more signaling domains (e.g., T cell activation domains). These special receptors allow the T cells to recognize a specific protein antigen on tumor cells and induce T cell activation and signaling pathways. Recent results of clinical trials with chimeric receptor-expressing T cells have provided compelling support of their utility as agents for cancer immunotherapy. However, despite these promising results, a number of side effects associated the CAR T-cell therapeutics were identified, raising significant safety concerns. One side effect is “on-target but off-tissue” adverse events from TCR and CAR engineered T cells, in which a CAR T cell binds to its ligand outside of the target tumor tissue and induces an immune response. Therefore, the ability to identify appropriate CAR targets is important to effectively targeting and treating the tumor without damaging normal cells that express the same target antigen.

Inhibitory chimeric antigen receptors (also known as iCARs) are protein constructions that inhibit or reduce immunomodulatory cell activity after binding their cognate ligands on a target cell. Current iCAR designs leverage PD-1 intracellular domains for inhibition, but have proven difficult to reproduce. Thus, alternative inhibitory domains for use in iCARs are needed.

### SUMMARY

Provided herein are chimeric inhibitory receptors comprising: an extracellular protein-binding domain; a transmembrane domain, wherein the transmembrane domain is operably linked to the extracellular protein-binding domain; and one or more intracellular signaling domains, wherein the one or more intracellular signaling domains are operably linked to the transmembrane domain, and wherein at least one of the one or more intracellular signaling domain is capable of preventing, attenuating, or inhibiting activation of a tumor-targeting chimeric receptor expressed on an immunomodulatory cell.

In some aspects, the one or more intracellular signaling domains are each derived from a protein selected from the group consisting of: SLAP1, SLAP2, Dok-1, Dok-2, LAIR1, GRB-2, CD200R, SIRPα, HAVR, GITR, PD-L1, KIR2DL1, KIR2DL2, KIR2DL3KIR3DL2, CD94, KLRG-1, CEACAM1, LIR2, LIR3, LIR5, SIGLEC-2, and SIGLEC-10.

In some aspects, the transmembrane domain is derived from the same protein as one of the one or more intracellular signaling domains.

In some aspects, the transmembrane domain further comprises at least a portion of an extracellular domain of the same protein.

In some aspects, the transmembrane domain is derived from a first protein and the one or more intracellular signaling domains are derived from a second protein that are distinct from the first protein.

In some aspects, one of the one or more intracellular signaling domains are derived from SLAP1.

In some aspects, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 4)
PAPAERPLPNPEGLDSDFLAVLSDYPSPDISPPIF
RRGEKLRVISDEGGWWKAISLSTGRESYIPGICVA
RVYHGWLFEGLGRDKAEELLQLPDTKVGSFMIRES
ETKKGFYSLSVRHRQVKHYRIFRLPNNWYYISPRL
TFQCLEDLVNHYSEVADGLCCVLTTPCLTQSTAAP
AVRASSSPVTLRQKTVDWRRVSRLQEDPEGTENPL
GVDESLFSYGLRESIASYLSLTSEDNTSFDRKKKS
ISLMYGGSKRKSSFFSSPPYFED,
or
(SEQ ID NO: 5)
PAPAERPLPNPEGLDSDFLA
VLSDYPSPDISPPIFRRGEKLRVISDEGGWWKAIS
LSTGRESYIPGICVARVYHGWLFEGLGRDKAEELL
QLPDTKVGSFMIRESETKKGFYSLSVRHRQVKHYR
IFRLPNNWYYISPRLTFQCLEDLVNHYSEVADGLC
CVLTTPCLTQSTAAPAVRASSSPVTLRQKTVDWRR
VSRLQEDPEGTENPLGVDESLFSYGLRESIASYLS
LTSEDNTSF.

In some aspects, the intracellular signaling domain comprises the amino acid sequence of

(SEQ ID NO: 4)
PAPAERPLPNPEGLDSDFLAVLSDYPSPDISPPIF
RRGEKLRVISDEGGWWKAISLSTGRESYIPGICVA
RVYHGWLFEGLGRDKAEELLQLPDTKVGSFMIRES
ETKKGFYSLSVRHRQVKHYRIFRLPNNWYYISPRL
TFQCLEDLVNHYSEVADGLCCVLTTPCLTQSTAAP
AVRASSSPVTLRQKTVDWRRVSRLQEDPEGTENPL
GVDESLFSYGLRESIASYLSLTSEDNTSFDRKKKS
ISLMYGGSKRKSSFFSSPPYFED,
or
(SEQ ID NO: 5)
PAPAERPLPNPEGLDSDFLAV
LSDYPSPDISPPIFRRGEKLRVISDEGGWWKAISL
STGRESYIPGICVARVYHGWLFEGLGRDKAEELLQ
LPDTKVGSFMIRESETKKGFYSLSVRHRQVKHYRI
FRLPNNWYYISPRLTFQCLEDLVNHYSEVADGLCC
VLTTPCLTQSTAAPAVRASSSPVTLRQKTVDWRRV
SRLQEDPEGTENPLGVDESLFSYGLRESIASYLSL
TSEDNTSF.

In some aspects, one of the one or more intracellular signaling domains is derived from SLAP2.

In some aspects, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 6)
RKSLPSPSLSSSVQGQGPVTMEAERSKATAVALGS
FPAGGPAELSLRLGEPLTIVSEDGDWWTVLSEVSG
REYNIPSVHVAKVSHGWLYEGLSREKAEELLLLPG
NPGGAFLIRESQTRRGSYSLSVRLSRPASWDRIRH
YRIHCLDNGWLYISPRLTFPSLQALVDHYSELADD
ICCLLKEPCVLQRAGPLPGKDIPLPVTVQRTPLNW
KELDSSLLFSEAATGEESLLSEGLRESLSFYISLN
DEAVSLDDA.

In some aspects, the intracellular signaling domain comprises the amino acid sequence of

(SEQ ID NO: 6)
RKSLPSPSLSSSVQGQGPVTMEAERSKATAVALGS
FPAGGPAELSLRLGEPLTIVSEDGDWWTVLSEVSG
REYNIPSVHVAKVSHGWLYEGLSREKAEELLLLPG
NPGGAFLIRESQTRRGSYSLSVRLSRPASWDRIRH
YRIHCLDNGWLYISPRLTFPSLQALVDHYSELADD
ICCLLKEPCVLQRAGPLPGKDIPLPVTVQRTPLNW
KELDSSLLFSEAATGEESLLSEGLRESLSFYISLN
DEAVSLDDA.

In some aspects, one of the one or more intracellular signaling domains is derived from KIR2DL1.

In some aspects, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 60)
HRWCSNKKNAAVMDQESAGNRTANSEDSDEQDPQE
VTYTQLNHCVFTQRKITRPSQRPKTPPTDIIVYTE
LPNAESRSKVVSCP.

In some aspects, the intracellular signaling domain comprises the amino acid sequence of

(SEQ ID NO: 60)
HRWCSNKKNAAVMDQESAGNRTANSEDSDEQDPQE
VTYTQLNHCVFTQRKITRPSQRPKTPPTDIIVYTE
LPNAESRSKVVSCP.

In some aspects, one of the one or more intracellular signaling domains is derived from KLRG-1.

In some aspects, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to MTDSVIYSMLELPTATQAQNDYGPQQKSSSSRPSCSCLGSG (SEQ ID NO: 61).

In some aspects, the intracellular signaling domain comprises the amino acid sequence of MTDSVIYSMLELPTATQAQNDYGPQQKSSSSRPSCSCLGSG (SEQ ID NO: 61).

In some aspects, one of the one or more intracellular signaling domains is derived from LAIR1.

In some aspects, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 62)
HRQNQIKQGPPRSKDEEQKPQQRPDLAVDVLERTA
DKATVNGLPEKDRETDTSALAAGSSQEVTYAQLDH
WALTQRTARAVSPQSTKPMAESITYAAVARH.

In some aspects, the intracellular signaling domain comprises the amino acid sequence of

(SEQ ID NO: 62)
HRQNQIKQGPPRSKDEEQKPQQRPDLAVDVLERTA
DKATVNGLPEKDRETDTSALAAGSSQEVTYAQLDH
WALTQRTARAVSPQSTKPMAESITYAAVARH.

In some aspects, one of the one or more intracellular signaling domains is derived from LIR2.

In some aspects, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 63)
LRHRRQGKHWTSTQRKADFQHPAGAVGPEPTDRGL
QWRSSPAADAQEENLYAAVKDTQPEDGVEMDTRAA
ASEAPQDVTYAQLHSLTLRRKATEPPPSQEREPPA
EPSIYATLAIH.

In some aspects, the intracellular signaling domain comprises the amino acid sequence of

(SEQ ID NO: 63)
LRHRRQGKHWTSTQRKADFQHPAGAVGPEPTDRGL
QWRSSPAADAQEENLYAAVKDTQPEDGVEMDTRAA
ASEAPQDVTYAQLHSLTLRRKATEPPPSQEREPPA
EPSIYATLAIH.

In some aspects, one of the one or more intracellular signaling domains is derived from LIR3.

In some aspects, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 64)
RRQRHSKHRTSDQRKTDFQRPAGAAETEPKDRGLLR
RSSPAADVQEENLYAAVKDTQSEDRVELDSQSPHD
EDPQAVTYAPVKHSSPRREMASPPSSLSGEFLDTK
DRQVEEDRQMDTEAAASEASQDVTYAQLHSLTLRR
KATEPPPSQEGEPPAEPSIYATLAIH.

In some aspects, the intracellular signaling domain comprises the amino acid sequence of

(SEQ ID NO: 64)
RRQRHSKHRTSDQRKTDFQRPAGAAETEPKDRGLL
RRSSPAADVQEENLYAAVKDTQSEDRVELDSQSPH
DEDPQAVTYAPVKHSSPRREMASPPSSLSGEFLDT
KDRQVEEDRQMDTEAAASEASQDVTYAQLHSLTLR
RKATEPPPSQEGEPPAEPSIYATLAIH.

In some aspects, wherein one of the one or more intracellular signaling domains is derived from LIR5.

In some aspects, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 65)
QHWRQGKHRTLAQRQADFQRPPGAAEPEPKDGGLQ
RRSSPAADVQGENFCAAVKNTQPEDGVEMDTRQSP
HDEDPQAVTYAKVKHSRPRREMASPPSPLSGEFLD
TKDRQAEEDRQMDTEAAASEAPQDVTYAQLHSFTL
RQKATEPPPSQEGASPAEPSVYATLAIH.

In some aspects, the intracellular signaling domain comprises the amino acid sequence of

(SEQ ID NO: 65)
QHWRQGKHRTLAQRQADFQRPPGAAEPEPKDGGLQ
RRSSPAADVQGENFCAAVKNTQPEDGVEMDTRQSP
HDEDPQAVTYAKVKHSRPRREMASPPSPLSGEFLD
TKDRQAEEDRQMDTEAAASEAPQDVTYAQLHSFTL
RQKATEPPPSQEGASPAEPSVYATLAIH.

In some aspects, one of the one or more intracellular signaling domains is derived from SIGLEC-2.

In some aspects, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 66)
KLQRRWKRTQSQQGLQENSSGQSFFVRNKKVRRAP
LSEGPHSLGCYNPMMEDGISYTTLRFPEMNIPRTG
DAESSEMQRPPPDCDDTVTYSALHKRQVGDYENVI
PDFPEDEGIHYSELIQFGVGERPQAQENVDYVILK
H.

In some aspects, the intracellular signaling domain comprises the amino acid sequence of

(SEQ ID NO: 66)
KLQRRWKRTQSQQGLQENSSGQSFFVRNKKVRRAP
LSEGPHSLGCYNPMMEDGISYTTLRFPEMNIPRTG
DAESSEMQRPPPDCDDTVTYSALHKRQVGDYENVI
PDFPEDEGIHYSELIQFGVGERPQAQENVDYVILK
H.

In some aspects, one of the one or more intracellular signaling domains is derived from SIGLEC-10.

In some aspects, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 67)
KILPKRRTQTETPRPRFSRHSTILDYINVVPTAGP
LAQKRNQKATPNSPRTPLPPGAPSPESKKNQKKQY
QLPSFPEPKSSTQAPESQESQEELHYATLNFPGVR
PRPEARMPKGTQADYAEVKFQ.

In some aspects, the intracellular signaling domain comprises the amino acid sequence of

(SEQ ID NO: 67)
KILPKRRTQTETPRPRFSRHSTILDYINVVPTAGPLAQKRNQKATPNSPR
TPLPPGAPSPESKKNQKKQYQLPSFPEPKSSTQAPESQESQEELHYATLN
FPGVRPRPEARMPKGTQADYAEVKFQ.

In some aspects, the transmembrane domain is derived from a protein selected from the group consisting of: CD8, CD28, CD3ζ, CD4, 4-IBB, OX40, ICOS, 2B4, CD25, CD7, LAX, LAT, LAIR1, GRB-2, Dok-1, Dok-2, SLAP1, SLAP2, CD200R, SIRPα, HAVR, GITR, PD-L1, KIR2DL1, KIR2DL2, KIR2DL3, KIR3DL2, CD94, KLRG-1, CEACAM1, LIR2, LIR3, LIR5, SIGLEC-2, and SIGLEC-10.

In some aspects, the chimeric inhibitory receptor comprises a transmembrane domain derived from CD28.

In some aspects, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 20)
FWVLVVVGGVLACYSLLVTVAFIIFWV.

In some aspects, the transmembrane domain comprises the amino acid sequence of

(SEQ ID NO: 20)
FWVLVVVGGVLACYSLLVTVAFIIFWV.

In some aspects, the chimeric inhibitory receptor comprises a transmembrane domain derived from KIR2DL1.

In some aspects, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 76)
ILIGTSVVIILFILLFFLL.

In some aspects, the transmembrane domain comprises the amino acid sequence of

(SEQ ID NO: 76)
ILIGTSVVIILFILLFFLL.

In some aspects, the chimeric inhibitory receptor comprises a transmembrane domain derived from KLRG-1.

In some aspects, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 78)
VAIALGLLTAVLLSVLLYQWI.

In some aspects, the transmembrane domain comprises the amino acid sequence of

(SEQ ID NO: 78)
VAIALGLLTAVLLSVLLYQWI.

In some aspects, the chimeric inhibitory receptor comprises a transmembrane domain derived from LAIR1.

In some aspects, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 79)
ILIGVSVVFLFCLLLLVLFCL.

In some aspects, the transmembrane domain comprises the amino acid sequence of

(SEQ ID NO: 79)
ILIGVSVVFLFCLLLLVLFCL.

In some aspects, the chimeric inhibitory receptor comprises a transmembrane domain derived from LIR2.

In some aspects, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 80)
VIGILVAVVLLLLLLLLLFLI.

In some aspects, the transmembrane domain comprises the amino acid sequence of

(SEQ ID NO: 80)
VIGILVAVVLLLLLLLLLFLI.

In some aspects, the chimeric inhibitory receptor comprises a transmembrane domain derived from LIR3.

In some aspects, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 81)
VLIGVSVAFVLLLFLLLFLLL.

In some aspects, the transmembrane domain comprises the amino acid sequence of

(SEQ ID NO: 81)
VLIGVSVAFVLLLFLLLFLLL.

In some aspects, the chimeric inhibitory receptor comprises a transmembrane domain derived from LIR5.

In some aspects, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 82)
VLIGVLVVSILLLSLLLFLLL.

In some aspects, the transmembrane domain comprises the amino acid sequence of

(SEQ ID NO: 82)
VLIGVLVVSILLLSLLLFLLL.

In some aspects, the chimeric inhibitory receptor comprises a transmembrane domain derived from SIGLEC-2.

In some aspects, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 83)
VAVGLGSCLAILILAICGL.

In some aspects, the transmembrane domain comprises the amino acid sequence of

(SEQ ID NO: 83)
VAVGLGSCLAILILAICGL.

In some aspects, the chimeric inhibitory receptor comprises a transmembrane domain derived from SIGLEC-10.

In some aspects, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 84)
GAFLGIGITALLFLCLALIIM.

In some aspects, the transmembrane domain comprises the amino acid sequence of

(SEQ ID NO: 84)
GAFLGIGITALLFLCLALIIM.

In some aspects, the one or more intracellular signaling domains are two intracellular signaling domains.

In some aspects, the chimeric inhibitory receptor comprises a first intracellular signaling domain derived from KIR2DL1 and a second intracellular signaling domain derived from LIR2.

In some aspects, the chimeric inhibitory receptor comprises a first intracellular signaling domain derived from KIR2DL1 and a second intracellular signaling domain derived from LIR3.

In some aspects, the chimeric inhibitory receptor comprises a first intracellular signaling domain derived from KIR2DL1 and a second intracellular signaling domain derived from LIR5.

In some aspects, the first intracellular signaling domain further comprises a transmembrane domain derived from KIR2DL1.

In some aspects, the chimeric inhibitory receptor comprises a first intracellular signaling domain derived from LIR2 and a second intracellular signaling domain derived from KIR2DL1.

In some aspects, first intracellular signaling domain further comprises a transmembrane domain derived from LIR2.

In some aspects, the chimeric inhibitory receptor comprises a first intracellular signaling domain derived from LIR3 and a second intracellular signaling domain derived from KIR2DL1.

In some aspects, the first intracellular signaling domain further comprises a transmembrane domain derived from LIR3.

In some aspects, the chimeric inhibitory receptor comprises a first intracellular signaling domain derived from LIR5 and a second intracellular signaling domain derived from KIR2DL1.

In some aspects, the first intracellular signaling domain further comprises a transmembrane domain derived from LIR5.

In some aspects, the protein is not expressed on the target tumor.

In some aspects, the protein is expressed on a non-tumor cell.

In some aspects, the protein is expressed on a non-tumor cell derived from a tissue selected from the group consisting of brain, neuronal tissue, endocrine, endothelial, bone, bone marrow, immune system, muscle, lung, liver, gallbladder, pancreas, gastrointestinal tract, kidney, urinary bladder, male reproductive organs, female reproductive organs, adipose, soft tissue, and skin.

In some aspects, the extracellular protein binding domain comprises a ligand-binding domain.

In some aspects, the extracellular protein binding domain comprises a receptor-binding domain.

In some aspects, the extracellular protein binding domain comprises an antigen-binding domain.

In some aspects, the antigen-binding domain comprises an antibody, an antigen-binding fragment of an antibody, a F(ab) fragment, a F(ab′) fragment, a single chain variable fragment (scFv), or a single-domain antibody (sdAb).

In some aspects, the antigen-binding domain comprises a single chain variable fragment (scFv).

In some aspects, each scFv comprises a heavy chain variable domain (VH) and a light chain variable domain (VL).

In some aspects, the VH and VL are separated by a peptide linker.

In some aspects, the peptide linker comprises an amino acid sequence selected from the group consisting of: GGS (SEQ ID NO: 23), GGSGGS (SEQ ID NO: 24), GGSGGSGGS (SEQ ID NO: 25), GGSGGSGGSGGS (SEQ ID NO: 26), GGSGGSGGSGGSGGS (SEQ ID NO: 27), GGGS (SEQ ID NO: 28), GGGSGGGS (SEQ ID NO: 29), GGGSGGGSGGGS (SEQ ID NO: 30), GGGSGGGSGGGSGGGS (SEQ ID NO: 31), GGGSGGGSGGGSGGGSGGGS (SEQ ID NO: 32), GGGGS (SEQ ID NO: 33), GGGGSGGGGS (SEQ ID NO: 34), GGGGSGGGGSGGGGS (SEQ ID NO: 35), GGGGSGGGGSGGGGSGGGGS (SEQ ID NO: 36), GGGGSGGGGSGGGGSGGGGSGGGGS (SEQ ID NO: 37), and TTTPAPRPPTPAPTIALQPLSLRPEACRPAAGGAVHTRGLDFACDQTTPGERSSLPAFY PGTSGSCSGCGSLSLP (SEQ ID NO: 94).

In some aspects, the scFv comprises the structure VH-L-VL or VL-L-VH, wherein VH is the heavy chain variable domain, L is the peptide linker, and VL is the light chain variable domain.

In some aspects, the transmembrane domain is physically linked to the extracellular protein-binding domain.

In some aspects, one of the one or more intracellular signaling domain is physically linked to the transmembrane domain.

In some aspects, the transmembrane domain is physically linked to the extracellular protein binding domain and one of the one or more intracellular signaling domains is physically linked to the transmembrane domain.

In some aspects, the extracellular protein binding has a high binding affinity.

In some aspects, the extracellular protein binding has a low binding affinity.

In some aspects, the chimeric inhibitory receptor is capable of suppressing cytokine production from an activated immunomodulatory cell.

In some aspects, the chimeric inhibitory receptor is capable of suppressing a cell-mediated immune response to a target cell, wherein the immune response is induced by activation of the immunomodulatory cell.

In some aspects, the target cell is a tumor cell.

In some aspects, the one or more intracellular signaling domains comprises one or more modifications.

In some aspects, the one or more modifications modulate sensitivity of the chimeric inhibitory receptor relative to the otherwise identical, unmodified receptor.

In some aspects, the one or more modifications increase sensitivity of the chimeric inhibitory receptor relative to the otherwise identical, unmodified receptor.

In some aspects, the one or more modifications reduce sensitivity of the chimeric inhibitory receptor relative to the otherwise identical, unmodified receptor.

In some aspects, the one or more modifications modulate potency of the chimeric inhibitory receptor relative to the otherwise identical, unmodified receptor.

In some aspects, the one or more modifications increase potency of the chimeric inhibitory receptor relative to the otherwise identical, unmodified receptor.

In some aspects, the one or more modifications reduce potency of the chimeric inhibitory receptor relative to the otherwise identical, unmodified receptor.

In some aspects, the one or more modifications modulate basal prevention, attenuation, or inhibition of activation of the tumor-targeting chimeric receptor when expressed on an immunomodulatory cell relative to the otherwise identical, unmodified receptor.

In some aspects, the one or more modifications reduce basal prevention, attenuation, or inhibition relative to the otherwise identical, unmodified receptor.

In some aspects, the one or more modifications increase basal prevention, attenuation, or inhibition relative to the otherwise identical, unmodified receptor.

In some aspects, the chimeric inhibitory receptor further comprises a spacer region positioned between the extracellular protein binding domain and the transmembrane domain and operably linked to each of the extracellular protein-binding domain and the transmembrane domain.

In some aspects, the chimeric inhibitory receptor further comprises a spacer region positioned between the extracellular protein binding domain and the transmembrane domain and physically linked to each of the extracellular protein binding domain and the transmembrane domain.

In some aspects, the spacer region is derived from a protein selected from the group consisting of: CD8α, CD4, CD7, CD28, IgG1, IgG4, FcγRIIIα, LNGFR, and PDGFR.

In some aspects, the spacer region comprises an amino acid sequence selected from the group consisting of:

(SEQ ID NO: 39)
AAAIEVMYPPPYLDNEKSNGTIIHVKGKHLCPSPLFPGPSKP,
(SEQ ID NO: 40)
ESKYGPPCPSCP,
(SEQ ID NO: 41)
ESKYGPPAPSAP,
(SEQ ID NO: 42)
ESKYGPPCPPCP,
(SEQ ID NO: 43)
EPKSCDKTHTCP,
(SEQ ID NO: 44)
AAAFVPVFLPAKPTTTPAPRPPTPAPTIASQPLSLRPEACRPAAGGAV
HTRGLDFACDIYIWAPLAGTCGVLLLSLVITLYCNHRN,
(SEQ ID NO: 46)
ACPTGLYTHSGECCKACNLGEGVAQPCGANQTVCEPCLDSVTFSDVVS
ATEPCKPCTECVGLQSMSAPCVEADDAVCRCAYGYYQDETTGRCEACR
VCEAGSGLVFSCQDKQNTVCEECPDGTYSDEADAEC,
(SEQ ID NO: 47)
ACPTGLYTHSGECCKACNLGEGVAQPCGANQTVC,
and
(SEQ ID NO: 48)
AVGQDTQEVIVVPHSLPFKV.

In some aspects, the spacer region modulates sensitivity of the chimeric inhibitory receptor relative to an otherwise identical chimeric inhibitory receptor lacking the spacer region.

In some aspects, the spacer region increases sensitivity of the chimeric inhibitory receptor relative to an otherwise identical chimeric inhibitory receptor lacking the spacer region.

In some aspects, the spacer region reduces sensitivity of the chimeric inhibitory receptor relative to an otherwise identical chimeric inhibitory receptor lacking the spacer region.

In some aspects, the spacer region modulates potency of the chimeric inhibitory receptor relative to an otherwise identical chimeric inhibitory receptor lacking the spacer region.

In some aspects, the spacer region increases potency of the chimeric inhibitory receptor relative to an otherwise identical chimeric inhibitory receptor lacking the spacer region.

In some aspects, the spacer region reduces potency of the chimeric inhibitory receptor relative to an otherwise identical chimeric inhibitory receptor lacking the spacer region.

In some aspects, the spacer region modulates basal prevention, attenuation, or inhibition of activation of the tumor-targeting chimeric receptor when expressed on an immunomodulatory cell relative to an otherwise identical chimeric inhibitory receptor lacking the spacer region.

In some aspects, the spacer region reduces basal prevention, attenuation, or inhibition relative to an otherwise identical chimeric inhibitory receptor lacking the spacer region.

In some aspects, the spacer region increases basal prevention, attenuation, or inhibition relative to an otherwise identical chimeric inhibitory receptor lacking the spacer region.

In some aspects, the chimeric inhibitory receptor further comprises an intracellular spacer region positioned between the transmembrane domain and one of the one or more intracellular signaling domains and operably linked to each of the transmembrane domain and one of the one or more intracellular signaling domains.

In some aspects, the chimeric inhibitory receptor further comprises an intracellular spacer region positioned between the transmembrane domain and one of the one or more intracellular signaling domains and physically linked to each of the transmembrane domain and one of the one or more intracellular signaling domains.

In some aspects, the intracellular spacer region modulates sensitivity of the chimeric inhibitory receptor relative to an otherwise identical chimeric inhibitory receptor lacking the intracellular spacer region.

In some aspects, the intracellular spacer region increases sensitivity of the chimeric inhibitory receptor relative to an otherwise identical chimeric inhibitory receptor lacking the intracellular spacer region.

In some aspects, the intracellular spacer region reduces sensitivity of the chimeric inhibitory receptor relative to an otherwise identical chimeric inhibitory receptor lacking the intracellular spacer region.

In some aspects, the intracellular spacer region modulates potency of the chimeric inhibitory receptor relative to an otherwise identical chimeric inhibitory receptor lacking the intracellular spacer region.

In some aspects, the intracellular spacer region increases potency of the chimeric inhibitory receptor relative to an otherwise identical chimeric inhibitory receptor lacking the intracellular spacer region.

In some aspects, the intracellular spacer region reduces potency of the chimeric inhibitory receptor relative to an otherwise identical chimeric inhibitory receptor lacking the intracellular spacer region.

In some aspects, the intracellular spacer region modulates basal prevention, attenuation, or inhibition of activation of the tumor-targeting chimeric receptor when expressed on an immunomodulatory cell relative to an otherwise identical chimeric inhibitory receptor lacking the intracellular spacer region.

In some aspects, the intracellular spacer region reduces basal prevention, attenuation, or inhibition relative to an otherwise identical chimeric inhibitory receptor lacking the intracellular spacer region.

In some aspects, the intracellular spacer region increases basal prevention, attenuation, or inhibition relative to an otherwise identical chimeric inhibitory receptor lacking the intracellular spacer region.

In some aspects, the inhibitory chimeric receptor further comprises an enzymatic inhibitory domain.

In some aspects, the enzymatic inhibitory domain is capable of preventing, attenuating, or inhibiting activation of a tumor-targeting chimeric receptor when expressed on an immunomodulatory cell relative to an otherwise identical chimeric inhibitory receptor lacking the enzymatic inhibitory domain.

In some aspects, the enzymatic inhibitory domain comprises an enzyme catalytic domain.

In some aspects, the enzyme catalytic domain is derived from an enzyme selected from the group consisting of: CSK, SHP-1, PTEN, CD45, CD148, PTP-MEG1, PTP-PEST, c-CBL, CBL-b, PTPN22, LAR, PTPH1, SHIP-1, and RasGAP.

In some aspects, the enzymatic inhibitory domain comprises one or more modifications that modulate basal prevention, attenuation, or inhibition.

In some aspects, the one or more modifications reduce basal prevention, attenuation, or inhibition relative to an otherwise identical enzymatic inhibitory domain lacking the one or more modifications.

In some aspects, the one or more modifications increase basal prevention, attenuation, or inhibition relative to an otherwise identical enzymatic inhibitory domain lacking the one or more modifications.

In some aspects, the tumor-targeting chimeric receptor is a chimeric antigen receptor (CAR) or an engineered T cell receptor (TCR).

In some aspects, the immunomodulatory cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell.

In some aspects, the immunomodulatory cell is a Natural Killer (NK) cell.

Also provided herein are compositions comprising the chimeric inhibitory receptor as described herein and a pharmaceutically acceptable carrier.

Also provided herein are engineered nucleic acids encoding the chimeric inhibitory receptor as described herein.

Also provided herein are expression vectors comprising the engineered nucleic acid as described herein.

Also provided herein are composition comprising the engineered nucleic acid as described herein or the expression vector as described herein, and a pharmaceutically acceptable carrier

Also provided herein are isolated immunomodulatory cells comprising the chimeric inhibitory receptor as described herein.

In some aspects, the cell further comprises a tumor-targeting chimeric receptor expressed on the surface of the cell.

In some aspects, upon binding of the protein to the chimeric inhibitory receptor, the chimeric inhibitory receptor prevents, attenuates, or inhibits activation of the tumor-targeting chimeric receptor relative to an otherwise identical cell lacking a chimeric inhibitory receptor.

Also provided herein are isolated immunomodulatory cells comprising a chimeric inhibitory receptor, wherein the chimeric inhibitory receptor comprises: an extracellular protein binding domain, a transmembrane domain, wherein the transmembrane domain is operably linked to the extracellular protein binding domain, and one or more intracellular signaling domains, wherein the one or more intracellular signaling domains is operably linked to the transmembrane domain, and wherein the one or more intracellular signaling domain are each derived from a protein selected from the group consisting of: SLAP1, SLAP2, Dok-1, Dok-2, LAIR1, GRB-2, CD200R, SIRPα, HAVR, GITR, PD-L1, KIR2DL1, KIR2DL2, KIR2DL3, KIR3DL2, CD94, KLRG-1, CEACAM1, LIR2, LIR3, LIR5, SIGLEC-2, and SIGLEC-10; and wherein upon binding of the protein to the chimeric inhibitory receptor, the chimeric inhibitory receptor prevents, attenuates, or inhibits activation of a tumor-targeting chimeric receptor expressed on the surface of the cell.

In some aspects, the cell further comprises a tumor-targeting chimeric receptor expressed on the surface of the cell.

Also provided herein are isolated cells comprising: a chimeric inhibitory receptor, wherein and the chimeric inhibitory receptor comprises: an extracellular protein binding domain, a transmembrane domain, wherein the transmembrane domain is operably linked to the extracellular protein binding domain, and one or more intracellular signaling domains, wherein the one or more intracellular signaling domains are operably linked to the transmembrane domain, and wherein the one or more intracellular signaling domain are each derived from a protein selected from the group consisting of: SLAP1, SLAP2, Dok-1, Dok-2, LAIR1, GRB-2, CD200R, SIRPα, HAVR, GITR, PD-L1, KIR2DL1, KIR2DL2, KIR2DL3, KIR3DL2, CD94, KLRG-1, CEACAM1, LIR2, LIR3, LIR5, SIGLEC-2, and SIGLEC-10; and a tumor-targeting chimeric receptor expressed on the surface of the cell, wherein upon binding of the protein to the chimeric inhibitory receptor, the chimeric inhibitory receptor prevents, attenuates, or inhibits activation of the tumor-targeting chimeric receptor.

In some aspects, the chimeric inhibitory receptor is recombinantly expressed.

In some aspects, the chimeric inhibitory receptor is expressed from a vector or a selected locus from the genome of the cell.

In some aspects, the tumor-targeting chimeric receptor is a chimeric antigen receptor (CAR) or an engineered T cell receptor.

In some aspects, prior to binding of the protein to the chimeric inhibitory receptor, the tumor-targeting chimeric receptor is capable of activating the cell.

In some aspects, upon binding of the protein to the chimeric inhibitory receptor, the chimeric inhibitory receptor suppresses cytokine production from the activated cell.

In some aspects, upon binding of the protein to the chimeric inhibitory receptor, the chimeric inhibitory receptor suppresses a cell-mediated immune response to a target cell, wherein the immune response is induced by activation of the immunomodulatory cell.

In some aspects, the transmembrane domain is physically linked to the extracellular protein binding domain.

In some aspects, the intracellular signaling domain is physically linked to the transmembrane domain.

In some aspects, the transmembrane domain is physically linked to the extracellular protein binding domain and one of the one or more intracellular signaling domains is physically linked to the transmembrane domain.

In some aspects, the target cell is a tumor cell.

In some aspects, the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell.

In some aspects, the immunomodulatory cell is a Natural Killer (NK) cell.

In some aspects, the cell is autologous.

In some aspects, the cell is allogeneic.

Also provided herein are compositions comprising the isolated cell as described herein and a pharmaceutically acceptable carrier.

Also provided herein are methods of preventing, attenuating, or inhibiting a cell-mediated immune response induced by a tumor-targeting chimeric receptor expressed of the surface of an immunomodulatory cell, comprising: engineering the immunomodulatory cell to express the chimeric inhibitory receptor of any one of claims 1-75 on the surface of the immunomodulatory cell, wherein upon binding of a cognate antigen to the chimeric inhibitory receptor, the intracellular signaling domain prevents, attenuates, or inhibits activation of the tumor-targeting chimeric receptor.

Also provided herein are methods of preventing, attenuating, or inhibiting activation of a tumor-targeting chimeric receptor expressed on the surface of an immunomodulatory cell, comprising: contacting the isolated cell as described herein or the composition as described herein with a cognate antigen of the chimeric inhibitory receptor under conditions suitable for the chimeric inhibitory receptor to bind the cognate antigen, wherein upon binding of the antigen to the chimeric inhibitory receptor, the intracellular signaling domain prevents, attenuates, or inhibits activation of the tumor-targeting chimeric receptor.

In some aspects, the tumor-targeting chimeric receptor is a chimeric antigen receptor (CAR) or an engineered T cell receptor.

In some aspects, the CAR binds one or more antigens expressed on the surface of a tumor cell.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1A shows an exemplary diagram of a T cell co-expressing an anti-CD19-SLAP iCAR and an anti-CD20-CD28/CD3ζ aCAR contacting a target cell expressing CD19 and CD20. FIG. 1B shows negative control cells with no expression of either CAR construct.

FIG. 1C shows anti-CD20-CD28/CD3ζ aCAR expression in transduced T cells. FIG. 1D shows anti-CD20-CD28/CD3 aCAR and anti-CD19-SLAP iCAR expression in transduced T cells.

FIG. 2A shows TNF-α production by T cells is reduced by co-expression of an anti-CD20 aCAR and an anti-CD19 iCAR as compared to an anti-CD20 aCAR alone. FIG. 2B shows IFN-γ production by T cells is reduced by co-expression of an anti-CD20 aCAR and an anti-CD19 iCAR as compared to an anti-CD20 aCAR alone. FIG. 2C shows IL-2 production by T cells is reduced by co-expression of an anti-CD20 aCAR and an anti-CD19 iCAR as compared to an anti-CD20 aCAR alone.

FIG. 3 shows expression profiles of an anti-FLT3 aCAR and various iCAR formats with an anti-EMCN binding domain, including co-expression, following transduction of NK cells as assessed by flow cytometry. Between 1 and 3 biological replicates per condition (indicated as separate points).

FIG. 4 shows NK cell mediated killing (top panels) and cytokine secretion (bottom panel). Shown are for the various NK cells engineered to co-express an anti-FLT3 aCAR and the indicated anti-EMCN iCARs. “Separate”=each type of SEM cell presented separately (top left panel). “Mixed”=both types of SEM cells mixed together in the same culture (top right panel). Between 1 and 3 biological replicates per condition (indicated as separate points). 3 technical replicates per measurement, X and Y SEM plotted where relevant. KLRG1 is not shown where its iCAR protection is negative.

### DETAILED DESCRIPTION

### Definitions

Terms used in the claims and specification are defined as set forth below unless otherwise specified.

The term “inhibitory chimeric receptor” or “inhibitory chimeric antigen receptor” or “chimeric inhibitory receptor” as used herein refers to a polypeptide or a set of polypeptides, which when expressed in an immune effector cell, provides the cell with specificity for a target cell, and with inhibitory intracellular signal generation. Inhibitory chimeric receptors typically include an extracellular protein binding domain (e.g., antibody fragment as an antigen-binding domain), a spacer domain, a transmembrane domain, and one or more intracellular signaling/co-signaling domains. An inhibitory chimeric receptor may also be called an “iCAR.”

The term “tumor targeting chimeric receptor” refers to activating chimeric receptors, tumor-targeting chimeric antigen receptors (CARs), or engineered T cell receptors. A tumor targeting chimeric receptor may also be called an “aCAR.”

The term “chimeric antigen receptor” or alternatively a “CAR” as used herein refers to a polypeptide or a set of polypeptides, which when expressed in an immune effector cell, provides the cell with specificity for a target cell, and with intracellular signal generation. CARs typically include an extracellular protein binding domain (e.g., antibody fragment as an antigen-binding domain), a spacer domain, a transmembrane domain, and one or more intracellular signaling/co-signaling domains. In some embodiments, a CAR comprises at least an extracellular antigen binding domain, a transmembrane domain and a cytoplasmic signaling domain (also referred to herein as “an intracellular signaling domain”) comprising a functional signaling domain derived from a inhibitory molecule or a stimulatory molecule and/or costimulatory molecule. In some aspects, the set of polypeptides that comprise the inhibitory chimeric receptor or tumor targeting chimeric receptor are contiguous with each other. In some embodiments, the inhibitory chimeric receptor or tumor targeting chimeric receptor further comprises a spacer domain between the extracellular antigen binding domain and the transmembrane domain. In some embodiments, the set of polypeptides include recruitment domains, such as dimerization or multimerization domains, that can couple the polypeptides to one another. In some embodiments, an inhibitory chimeric receptor comprises a chimeric fusion protein comprising an extracellular antigen binding domain, a transmembrane domain and an intracellular signaling domain comprising a functional signaling domain derived from an inhibitory molecule or a stimulatory molecule. In one aspect, an inhibitory chimeric receptor comprises a chimeric fusion protein comprising an extracellular antigen binding domain, a transmembrane domain and an intracellular signaling domain comprising a functional inhibitory domain derived from an inhibitory molecule. In one aspect, a tumor targeting chimeric receptor comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule.

The term, “intracellular signaling domain” as used herein, refers to a functional domain of the inhibitory chimeric receptor or the tumor targeting chimeric receptor located inside the cell. In some embodiments, the intracellular signaling domain is an inhibitory signaling domain. Following binding of the molecular binding domain to an protein, for example, an inhibitory signaling domain represses receptor signaling while an activation signaling domain transmits a signal (e.g., proliferative/survival signal) to the cell.

The term, “transmembrane domain” as used herein, refers to a domain that spans a cellular membrane. In some embodiments, a transmembrane domain comprises a hydrophobic alpha helix.

The term, “extracellular protein binding domain” or “extracellular antigen binding domain” as used herein, refers to a molecular binding domain which is typically an ectodomain of a cell receptor or the antigen binding domains of an antibody and is located outside the cell, exposed to the extracellular space. An extracellular antigen binding domain can include any molecule (e.g., protein or peptide) capable of binding to another protein or peptide. In some embodiments, an extracellular protein or antigen binding domain comprises an antibody, an antigen-binding fragment thereof, F(ab), F(ab′), a single chain variable fragment (scFv), or a single-domain antibody (sdAb). In some embodiments, an extracellular protein or antigen binding domain binds to a cell-surface ligand (e.g., an antigen, such as a cancer antigen or a protein expressed on the surface of a cell).

The term “tumor” refers to tumor cells and the associated tumor microenvironment (TME). In some embodiments, tumor refers to a tumor cell or tumor mass. In some embodiments, tumor refers to the tumor microenvironment.

The term “not expressed” refers to expression that is at least 2-fold lower than the level of expression in non-tumor cells that would result in activation of the tumor-targeting chimeric antigen receptor. In some embodiments, the expression is at least 2-fold, at least 3-fold, at least 4-fold, at least 5-fold, at least 6-fold, at least 7-fold, at least 8-fold, at least 9-fold, or at least 10-fold or more lower than the level of expression in non-tumor cells that would result in activation of the tumor-targeting chimeric antigen receptor.

The term “ameliorating” refers to any therapeutically beneficial result in the treatment of a disease state, e.g., a cancer disease state, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

It must be noted that, as used in the specification and the appended claims, the singular forms “a,” “an” and “the” include plural referents unless the context clearly dictates otherwise.

Chimeric Inhibitory Receptors

In one aspect, provided herein are chimeric inhibitory receptors comprising (i) an extracellular protein binding domain; (ii) a transmembrane domain, wherein the transmembrane domain is operably linked to the extracellular protein binding domain; and (iii) one or more intracellular signaling domains, wherein the one or more intracellular signaling domains are operably linked to the transmembrane domain, and wherein at least one of the one or more intracellular signaling domains is capable of preventing, attenuating, or inhibiting activation of a tumor-targeting chimeric receptor expressed on an immunomodulatory cell.

Generally, an inhibitory or tumor targeting chimeric receptor is designed for a T cell, or NK cell, and is a chimera of an intracellular signaling domain and an antigen-recognizing domain (e.g., a single chain fragment (scFv) of an antibody) (Enblad et al., Human Gene Therapy. 2015; 26(8):498-505). A T cell that expresses a chimeric antigen receptor (CAR) is known in the art as a CAR T cell. An activating or tumor targeting CAR generally induces T cell signaling pathways upon binding to its cognate ligand via an intracellular signaling domain that results in activation of the T cell and an immune response. Activation CAR, activating CAR, and tumor-targeting CAR are interchangeable terms.

An inhibitory chimeric receptor, generally, is an artificial immune cell receptor engineered to recognize and bind to proteins expressed by cells. Inhibitory chimeric receptors generally recognize proteins that are not expressed on tumor cells, while activating or tumor targeting chimeric receptors (e.g., aCARs) generally recognize proteins that are expressed on tumor cells. Chimeric receptors in general typically include an antibody fragment as an extracellular protein binding domain, a spacer or hinge domains, a hydrophobic alpha helix transmembrane domain, and one or more intracellular signaling/co-signaling domains.

An inhibitory chimeric receptor generally follows the structure of activating CARs (aCARs) but uses an inhibitory domain for the intracellular signaling domain, instead of an activation signaling domain derived from a T-cell receptor (TCR). The intracellular signaling/co-signaling domain are inhibitory domains that reduce or inhibit signaling by other receptor proteins in the same cell. An inhibitory chimeric receptor cell can contain an antigen-specific inhibitory receptor, for example, to block nonspecific immunoactivation, which may result from extra-tumor target expression. In some embodiments, an inhibitory chimeric receptor blocks T cell responses in T cells activated by either their endogenous T cell receptor or an activating or tumor-targeting CAR. For example, an immunomodulatory cell can express both an inhibitory chimeric receptor that recognizes a non-tumor protein target and a tumor-targeting chimeric receptor that recognizes a tumor protein. When such an immunomodulatory cell contacts a tumor cell, only the tumor-targeting receptor recognizes and binds its cognate ligand and is activated, resulting in induction of cell signaling pathways and immune cell activation. In contrast, when the immunomodulatory cell contacts a non-tumor target, the inhibitory chimeric receptor binds to its cognate ligand and represses or inhibits any signaling induced by the activation of the tumor-targeting chimeric receptor. Thus, the immunomodulatory cell can be constructed so that immune signaling only occurs when the cell contacts tumor cells.

In some embodiments, the protein bound by the inhibitory chimeric receptor is not expressed on the target tumor. In some embodiments, the expression is at least 2-fold, at least 3-fold, at least 4-fold, at least 5-fold, at least 6-fold, at least 7-fold, at least 8-fold, at least 9-fold, or at least 10-fold or more lower than the level of expression in non-tumor cells that would result in activation of the tumor-targeting chimeric antigen receptor.

In some embodiments, the protein bound by the inhibitory chimeric receptor is expressed on a non-tumor cell.

In some embodiments, the protein bound by the inhibitory chimeric receptor is expressed on a non-tumor cell derived from a tissue selected from the group consisting of brain, neuronal tissue, endocrine, endothelial, bone, bone marrow, immune system, muscle, lung, liver, gallbladder, pancreas, gastrointestinal tract, kidney, urinary bladder, male reproductive organs, female reproductive organs, adipose, soft tissue, and skin.

Intracellular Signaling Domains

The inhibitory chimeric receptors of the present disclosure comprise intracellular signaling domains that are capable of preventing, attenuating, or inhibiting activation of a tumor-targeting chimeric receptor expressed on an immunomodulatory cell. In some embodiments, the chimeric inhibitory receptor comprises one or more intracellular signaling domains.

In some embodiments, the intracellular signaling domain comprises one or more modifications. In some embodiments, the one or more modifications modulate sensitivity of the chimeric inhibitory receptor relative to the otherwise identical, unmodified receptor. In some embodiments, the one or more modifications increase sensitivity of the chimeric inhibitory receptor relative to the otherwise identical, unmodified receptor. In some embodiments, the one or more modifications reduce sensitivity of the chimeric inhibitory receptor relative to the otherwise identical, unmodified receptor. In some embodiments, the one or more modifications modulate potency of the chimeric inhibitory receptor relative to the otherwise identical, unmodified receptor. In some embodiments, the one or more modifications increase potency of the chimeric inhibitory receptor relative to the otherwise identical, unmodified receptor. In some embodiments, the one or more modifications reduce potency of the chimeric inhibitory receptor relative to the otherwise identical, unmodified receptor.

In some embodiments, the one or more modifications modulate basal prevention, attenuation, or inhibition of activation of the tumor-targeting chimeric receptor expressed on an immunomodulatory cell relative to the otherwise identical, unmodified receptor. In some embodiments, the one or more modifications reduce basal prevention, attenuation, or inhibition relative to the otherwise identical, unmodified receptor. In some embodiments, the one or more modifications increase basal prevention, attenuation, or inhibition relative to the otherwise identical, unmodified receptor.

Inhibitory Domains

In some embodiments, the inhibitory intracellular signaling domain is derived from a protein selected from the group consisting of: SLAP1, SLAP2, LAIR1, GRB-2, Dok-1, Dok-2, CD200R, SIRPalpha (SIRPα), HAVR, GITR, PD-L1, KIR2DL1, KIR2DL2, KIR2DL3, KIR3DL2, CD94, KLRG-1, CEACAM1, LIR2, LIR3, LIR5, SIGLEC-2, and SIGLEC-10. In some embodiments, the inhibitory chimeric receptor described herein comprises an inhibitory intracellular signaling domain. In some embodiments, the inhibitory intracellular signaling domain is a SLAP1 domain. In some embodiments, the SLAP1 domain comprises amino acid residues 8-276 of the full length SLAP1 protein. In some embodiments, the SLAP1 domain comprises amino acid residues 8-247 of the full length SLAP1 protein. In some embodiments, the SLAP1 domain comprises amino acid residues 8-261 of the full length SLAP1 protein. In some embodiments, the inhibitory intracellular signaling domain is a SLAP2 domain. In some embodiments, the inhibitory intracellular signaling domain is a Dok-2 domain. In some embodiments, the inhibitory intracellular signaling domain is a Dok-1 domain. In some embodiments, the inhibitory intracellular signaling domain is a GRB2 domain. In some embodiments, the inhibitory intracellular signaling domain is a CD200R domain. In some embodiments, the inhibitory intracellular signaling domain is a SIRPα domain.

Src-like adaptor proteins 1 and 2 (SLAP1 and SLAP2) are adaptor proteins involved in intracellular signaling pathways and are expressed in lymphocytes. Both SLAP1 and SLAP2 contain common SH2 and SH3 domains. SH2 domains allow proteins to bind to phosphorylated tyrosine epitopes. SLAP1 and SLAP2 function as negative regulators of T cell receptor (TCR) signaling, likely by associating with the E3 ubiquitin ligase c-Cbl, which promotes the ubiquitination and degradation of the TCR-chain, resulting in decreased TCR signaling.

Docking protein 2 (Dok-2) is part of a negative signaling complex in T cells. Docking protein 1 (Dok-1) is part of the negative regulation of the insulin receptor signaling pathway. Growth factor receptor-bound protein 2 (GRB2) is an adaptor protein involved in signal transduction and contains one SH2 domain and two SH3 domains. Signal-regulator protein alpha (SIRPα) is an inhibitory receptor that contains four immunoreceptor tyrosine-based inhibition motifs (ITIMs). Cell surface transmembrane glycoprotein CD200 receptor 1 (CD200R) is involved in signaling pathways that regulate the expression of pro-inflammatory molecules and associates with Dok-1 and Dok-2.

Exemplary inhibitory intracellular signaling domain amino acid sequences are shown in Table 1. Exemplary inhibitory intracellular signaling domain nucleic acid sequences are shown in Table 2.

TABLE 1
Exemplary inhibitory intracellular signaling domain amino acid sequences

| | | |
|---|---|---|
| | SEQ ID | |
| Amino Acid Sequence | NO: | Description |

Inhibitory Intracellular Signaling Domains

| | | |
|---|---|---|
| MEAIAKYDFKATADDELSFKRGDILKVLNEECDQNWYKAELNGK | 1 | GRB2 intracellular |
| DGFIPKNYIEMKPHPWFFGKIPRAKAEEMLSKQRHDGAFLIRESES | | signaling domain |
| APGDFSLSVKFGNDVQHFKVLRDGAGKYFLWVVKFNSLNELVDY | | |
| HRSTSVSRNQQIFLRDIEQVPQQPTYVQALFDFDPQEDGELGFRRG | | |
| DFIHVMDNSDPNWWKGACHGQTGMFPRNYVTPVNRNV | | |
| MDGAVMEGPLFLQSQRFGTKRWRKTWAVLYPASPHGVARLEFFD | 2 | Dok-1 intracellular |
| HKGSSSGGGRGSSRRLDCKVIRLAECVSVAPVTVETPPEPGATAFR | | signaling domain |
| LDTAQRSHLLAADAPSSAAWVQTLCRNAFPKGSWTLAPTDNPPKL | | |
| SALEMLENSLYSPTWEGSQFWVTVQRTEAAERCGLHGSYVLRVE | | |
| AERLTLLTVGAQSQILEPLLSWPYTLLRRYGRDKVMFSFEAGRRCP | | |
| SGPGTFTFQTAQGNDIFQAVETAIHRQKAQGKAGQGHDVLRADSH | | |
| EGEVAEGKLPSPPGPQELLDSPPALYAEPLDSLRIAPCPSQDSLYSD | | |
| PLDSTSAQAGEGVQRKKPLYWDLYEHAQQQLLKAKLTDPKEDPIY | | |
| DEPEGLAPVPPQGLYDLPREPKDAWWCQARVKEEGYELPYNPAT | | |
| DDYAVPPPRSTKPLLAPKPQGPAFPEPGTATGSGIKSHNSALYSQV | | |
| QKSGASGSWDCGLSRVGTDKTGVKSEGST | | |
| MGDGAVKQGFLYLQQQQTFGKKWRRFGASLYGGSDCALARLEL | 3 | Dok-2 intracellular |
| QEGPEKPRRCEAARKVIRLSDCLRVAEAGGEASSPRDTSAFFLETK | | signaling domain |
| ERLYLLAAPAAERGDWVQAICLLAFPGQRKELSGPEGKQSRPCME | | |
| ENELYSSAVTVGPHKEFAVTMRPTEASERCHLRGSYTLRAGESAL | | |
| ELWGGPEPGTQLYDWPYRFLRRFGRDKVTFSFEAGRRCVSGEGNF | | |
| EFETRQGNEIFLALEEAISAQKNAAPATPQPQPATIPASLPRPDSPYS | | |
| RPHDSLPPPSPTTPVPAPRPRGQEGEYAVPFDAVARSLGKNFRGILA | | |
| VPPQLLADPLYDSIEETLPPRPDHIYDEPEGVAALSLYDSPQEPRGE | | |
| AWRRQATADRDPAGLQHVQPAGQDFSASGWQPGTEYDNVVLKK | | |
| GPK | | |
| PAPAERPLPNPEGLDSDFLAVLSDYPSPDISPPIFRRGEKLRVISDEG | 4 | SLAP1ₐₐ₈₋ₐₐ₂₇₆ |
| GWWKAISLSTGRESYIPGICVARVYHGWLFEGLGRDKAEELLQLP | | intracellular signaling |
| DTKVGSFMIRESETKKGFYSLSVRHRQVKHYRIFRLPNNWYYISPR | | domain |
| LTFQCLEDLVNHYSEVADGLCCVLTTPCLTQSTAAPAVRASSSPVT | | |
| LRQKTVDWRRVSRLQEDPEGTENPLGVDESLFSYGLRESIASYLSL | | |
| TSEDNTSFDRKKKSISLMYGGSKRKSSFFSSPPYFED | | |
| PAPAERPLPNPEGLDSDFLAVLSDYPSPDISPPIFRRGEKLRVISDEG | 5 | SLAP1ₐₐ₈₋ₐₐ₂₄₇ |
| GWWKAISLSTGRESYIPGICVARVYHGWLFEGLGRDKAEELLQLP | | intracellular signaling |
| DTKVGSFMIRESETKKGFYSLSVRHRQVKHYRIFRLPNNWYYISPR | | domain |
| LTFQCLEDLVNHYSEVADGLCCVLTTPCLTQSTAAPAVRASSSPVT | | |
| LRQKTVDWRRVSRLQEDPEGTENPLGVDESLFSYGLRESIASYLSL | | |
| TSEDNTSF | | |
| RKSLPSPSLSSSVQGQGPVTMEAERSKATAVALGSFPAGGPAELSL | 6 | SLAP2ₐₐ₈₋ₐₐ₂₆₁ |
| RLGEPLTIVSEDGDWWTVLSEVSGREYNIPSVHVAKVSHGWLYEG | | intracellular signaling |
| LSREKAEELLLLPGNPGGAFLIRESQTRRGSYSLSVRLSRPASWDRI | | domain |
| RHYRIHCLDNGWLYISPRLTFPSLQALVDHYSELADDICCLLKEPC | | |
| VLQRAGPLPGKDIPLPVTVQRTPLNWKELDSSLLFSEAATGEESLLS | | |
| EGLRESLSFYISLNDEAVSLDDA | | |
| KVNGCRKYKLNKTESTPVVEEDEMQPYASYTEKNNPLYDTTNKV | 7 | CD200R intracellular |
| KASEALQSEVDTDLHTL | | signaling domain |
| RIRQKKAQGSTSSTRLHEPEKNAREITQDTNDITYADLNLPKGKKP | 8 | SIRPα intracellular |
| APQAAEPNNHTEYASIQTSPQPASEDTLTYADLDMVHLNRTPKQP | | signaling domain |
| APKPEPSFSEYASVQVPRK | | |
| HRWCSNKKNAAVMDQESAGNRTANSEDSDEQDPQEVTYTQLNH | 60 | KIR2DL1 intracellular |
| CVFTQRKITRPSQRPKTPPTDIIVYTELPNAESRSKVVSCP | | signaling domain |
| MTDSVIYSMLELPTATQAQNDYGPQQKSSSSRPSCSCLGSG | 61 | KLRG1 intracellular |
| | | signaling domain |
| HRQNQIKQGPPRSKDEEQKPQQRPDLAVDVLERTADKATVNGLPE | 62 | LAIR1 intracellular |
| KDRETDTSALAAGSSQEVTYAQLDHWALTQRTARAVSPQSTKPM | | signaling domain |
| AESITYAAVARH | | |
| LRHRRQGKHWTSTQRKADFQHPAGAVGPEPTDRGLQWRSSPAAD | 53 | LIR2 intracellular |
| AQEENLYAAVKDTQPEDGVEMDTRAAASEAPQDVTYAQLHSLTL | | signaling domain |
| RRKATEPPPSQEREPPAEPSIYATLAIH | | |
| RRQRHSKHRTSDQRKTDFQRPAGAAETEPKDRGLLRRSSPAADVQ | 64 | LIR3 intracellular |
| EENLYAAVKDTQSEDRVELDSQSPHDEDPQAVTYAPVKHSSPRRE | | signaling domain |
| MASPPSSLSGEFLDTKDRQVEEDRQMDTEAAASEASQDVTYAQLH | | |
| SLTLRRKATEPPPSQEGEPPAEPSIYATLAIH | | |
| QHWRQGKHRTLAQRQADFQRPPGAAEPEPKDGGLQRRSSPAADV | 65 | LIR5 intracellular |
| QGENFCAAVKNTQPEDGVEMDTRQSPHDEDPQAVTYAKVKHSRP | | signaling domain |
| RREMASPPSPLSGEFLDTKDRQAEEDRQMDTEAAASEAPQDVTYA | | |
| QLHSFTLRQKATEPPPSQEGASPAEPSVYATLAIH | | |
| KLQRRWKRTQSQQGLQENSSGQSFFVRNKKVRRAPLSEGPHSLGC | 66 | SIGLEC-2 intracellular |
| YNPMMEDGISYTTLRFPEMNIPRTGDAESSEMQRPPPDCDDTVTYS | | signaling domain |
| ALHKRQVGDYENVIPDFPEDEGIHYSELIQFGVGERPQAQENVDYV | | |
| ILKH | | |
| KILPKRRTQTETPRPRFSRHSTILDYINVVPTAGPLAQKRNQKATPN | 67 | SIGLEC-10 intracellular |
| SPRTPLPPGAPSPESKKNQKKQYQLPSFPEPKSSTQAPESQESQEEL | | signaling domain |
| HYATLNFPGVRPRPEARMPKGTQADYAEVKFQ | | |

TABLE 2
Exemplary inhibitory intracellular signaling domain nucleic acid sequences

| | | |
|---|---|---|
| | SEQ ID | |
| Nucleic Acid Sequence | NO: | Description |

Inhibitory Intracellular Signaling Domains

| | | |
|---|---|---|
| ATGGAAGCCATTGCCAAATACGACTTCAAGGCCACCGCCGACG | 9 | GRB2 intracellular |
| ACGAGCTGAGCTTCAAGAGAGGCGACATCCTGAAGGTGCTGAA | | signaling domain |
| CGAGGAATGCGACCAGAACTGGTACAAGGCCGAGCTGAACGGC | | |
| AAGGACGGCTTCATCCCCAAGAACTACATCGAGATGAAGCCCC | | |
| ATCCATGGTTCTTCGGCAAGATCCCCAGAGCCAAGGCCGAAGA | | |
| GATGCTGAGCAAGCAGAGACACGACGGCGCCTTTCTGATCCGG | | |
| GAATCTGAATCTGCCCCTGGCGACTTCAGCCTGAGCGTGAAGTT | | |
| CGGCAACGACGTGCAGCACTTCAAGGTCCTGAGAGATGGCGCC | | |
| GGAAAGTACTTCCTGTGGGTCGTGAAGTTTAACAGCCTGAACG | | |
| AGCTGGTGGACTACCACAGATCCACCAGCGTGTCCCGGAACCA | | |
| GCAGATCTTCCTGCGGGACATCGAACAGGTGCCACAGCAGCCA | | |
| ACATACGTGCAGGCCCTGTTCGACTTCGACCCTCAAGAGGATGG | | |
| CGAGCTGGGCTTTAGACGGGGCGATTTCATCCACGTGATGGAC | | |
| AACAGCGACCCCAACTGGTGGAAGGGCGCTTGTCATGGACAGA | | |
| CCGGCATGTTCCCCAGAAACTACGTGACCCCTGTGAACCGGAA | | |
| CGTG | | |
| ATGGATGGCGCCGTGATGGAAGGCCCTCTGTTTCTCCAGAGCCA | 10 | Dok-1 intracellular |
| GAGATTCGGCACCAAGCGGTGGCGGAAAACATGGGCCGTTCTG | | signaling domain |
| TACCCTGCCTCTCCTCATGGCGTGGCCCGGCTGGAATTTTTCGA | | |
| TCACAAGGGCTCTAGCAGCGGCGGAGGCAGAGGATCTAGTAGA | | |
| CGGCTGGACTGCAAAGTGATCCGGCTGGCCGAGTGTGTGTCTGT | | |
| GGCTCCTGTGACCGTGGAAACCCCTCCTGAACCTGGCGCCACAG | | |
| CCTTCAGACTGGATACAGCCCAGAGAAGCCATCTGCTGGCCGC | | |
| CGATGCTCCTTCTTCTGCTGCTTGGGTGCAGACCCTGTGCCGGA | | |
| ACGCTTTTCCTAAAGGCAGCTGGACACTGGCCCCTACCGACAAT | | |
| CCTCCTAAGCTGAGCGCCCTGGAAATGCTGGAAAACAGCCTGT | | |
| ACAGCCCCACCTGGGAGGGCTCTCAGTTTTGGGTCACCGTGCAG | | |
| AGAACAGAGGCCGCCGAAAGATGTGGCCTGCACGGCTCTTATG | | |
| TGCTGAGAGTGGAAGCCGAGAGACTGACCCTGCTGACAGTGGG | | |
| AGCCCAGTCTCAGATCCTGGAACCTCTGCTGAGCTGGCCCTACA | | |
| CACTGCTGAGAAGATACGGCCGGGACAAAGTGATGTTCAGCTT | | |
| CGAGGCCGGCAGAAGATGTCCTTCTGGCCCTGGCACATTCACAT | | |
| TTCAGACAGCCCAGGGCAACGACATCTTCCAGGCTGTGGAAAC | | |
| CGCCATCCACAGACAGAAGGCCCAGGGAAAAGCCGGCCAGGG | | |
| ACACGATGTTCTGAGAGCCGATTCTCACGAGGGCGAAGTGGCC | | |
| GAGGGAAAGCTTCCTTCTCCACCTGGACCTCAAGAGCTGCTGGA | | |
| TAGCCCTCCTGCTCTGTATGCCGAGCCTCTGGACAGCCTGAGAA | | |
| TCGCCCCTTGTCCAAGCCAGGACTCTCTGTACAGCGATCCCCTG | | |
| GATAGCACATCTGCCCAAGCTGGCGAAGGCGTGCAGAGGAAGA | | |
| AGCCCCTGTACTGGGATCTGTACGAGCACGCTCAGCAGCAACT | | |
| GCTGAAGGCCAAGCTGACAGACCCCAAAGAGGACCCCATCTAC | | |
| GACGAGCCTGAAGGACTTGCTCCAGTGCCACCTCAGGGCCTGT | | |
| ACGATCTGCCTAGAGAGCCTAAAGACGCCTGGTGGTGTCAGGC | | |
| CAGAGTGAAAGAGGAAGGCTACGAGCTGCCTTACAACCCCGCC | | |
| ACCGATGATTATGCCGTGCCTCCTCCAAGAAGCACCAAACCACT | | |
| GCTGGCCCCAAAGCCTCAGGGACCTGCTTTTCCTGAGCCTGGAA | | |
| CAGCCACAGGCAGCGGCATCAAGAGCCACAATAGCGCCCTGTA | | |
| TAGCCAGGTGCAGAAAAGCGGCGCCAGCGGCTCTTGGGATTGT | | |
| GGACTTAGCAGAGTGGGCACCGACAAGACCGGCGTGAAGTCTG | | |
| AGGGAAGCACA | | |
| ATGGGAGATGGCGCCGTGAAGCAGGGCTTTCTGTATCTCCAGC | 11 | Dok-2 intracellular |
| AGCAGCAGACCTTCGGCAAGAAGTGGCGGAGATTTGGCGCCTC | | signaling domain |
| TCTGTACGGCGGCTCTGATTGTGCTCTGGCCCGACTGGAACTGC | | |
| AAGAGGGACCTGAGAAGCCCAGAAGATGCGAGGCCGCCAGAA | | |
| AAGTGATCCGGCTGAGCGATTGTCTGAGAGTGGCTGAAGCAGG | | |
| CGGCGAAGCCAGCTCTCCTAGAGATACCAGCGCATTCTTCCTGG | | |
| AAACAAAAGAGCGGCTGTACCTGCTGGCCGCTCCTGCTGCTGA | | |
| AAGAGGCGATTGGGTCCAAGCCATCTGCCTGCTGGCTTTTCCCG | | |
| GCCAGAGAAAAGAGCTGTCTGGCCCTGAGGGCAAGCAGAGCAG | | |
| GCCTTGCATGGAAGAGAACGAGCTGTACTCCAGCGCCGTGACA | | |
| GTGGGCCCTCACAAAGAATTTGCCGTGACCATGAGGCCCACCG | | |
| AGGCCAGCGAAAGATGTCACCTGAGAGGCAGCTACACCCTGAG | | |
| AGCCGGCGAATCTGCTCTGGAACTTTGGGGAGGACCTGAGCCT | | |
| GGCACACAGCTGTACGACTGGCCCTACAGATTCCTGCGGAGATT | | |
| CGGCCGGGACAAAGTGACCTTCAGCTTTGAGGCTGGCCGCAGA | | |
| TGTGTGTCCGGCGAGGGCAATTTCGAGTTCGAGACAAGACAGG | | |
| GCAACGAGATCTTCCTGGCTCTGGAAGAGGCCATCAGCGCCCA | | |
| GAAAAATGCTGCCCCTGCTACACCTCAGCCTCAGCCTGCTACAA | | |
| TCCCTGCCTCTCTGCCCAGACCTGACAGCCCTTATAGCAGACCC | | |
| CACGACTCTCTGCCTCCACCTTCTCCAACAACACCCGTGCCTGC | | |
| TCCTAGACCTAGAGGACAAGAGGGCGAGTACGCCGTGCCTTTT | | |
| GATGCCGTGGCTAGAAGCCTGGGCAAGAACTTCAGAGGCATCC | | |
| TGGCTGTGCCTCCACAGCTGCTGGCTGACCCTCTGTACGACAGC | | |
| ATCGAGGAAACCCTGCCTCCAAGACCTGACCACATCTACGACG | | |
| AGCCTGAAGGCGTGGCAGCCCTGTCTCTGTATGACTCCCCTCAA | | |
| GAGCCTAGAGGCGAAGCCTGGCGTAGACAGGCTACCGCCGATA | | |
| GAGATCCTGCCGGACTGCAACATGTGCAGCCAGCCGGCCAGGA | | |
| TTTTTCTGCCTCTGGATGGCAGCCAGGCACCGAGTACGATAACG | | |
| TGGTGCTGAAGAAGGGCCCCAAG | | |
| CCAGCCCCAGCGGAGCGACCGCTGCCAAACCCTGAAGGGCTCG | 12 | SLAP1ₐₐ₈₋ₐₐ₂₇₆ |
| ACAGTGACTTTTTGGCTGTCCTCTCCGACTATCCTAGTCCCGATA | | intracellular signaling |
| TCAGCCCCCCGATATTCAGGCGCGGTGAAAAACTCCGAGTCATC | | domain |
| AGCGATGAGGGGGGTTGGTGGAAAGCCATCAGCCTGAGTACCG | | |
| GACGAGAGTCATACATTCCTGGAATATGTGTAGCGAGGGTGTA | | |
| CCACGGTTGGCTGTTCGAGGGTCTGGGAAGAGATAAGGCCGAG | | |
| GAACTTCTCCAACTCCCAGATACAAAAGTCGGTAGTTTTATGAT | | |
| TCGGGAAAGTGAAACTAAAAAGGGGTTCTATAGCCTCTCAGTT | | |
| CGGCATAGGCAGGTCAAGCATTATCGGATATTTCGCTTGCCTAA | | |
| CAACTGGTACTACATAAGTCCCCGACTCACATTCCAATGCCTGG | | |
| AGGACCTCGTGAATCACTATTCAGAAGTTGCAGATGGCCTCTGC | | |
| TGCGTACTCACGACACCCTGCCTTACCCAGAGTACAGCGGCCCC | | |
| GGCTGTTCGGGCATCTTCCAGCCCAGTAACACTCAGGCAAAAA | | |
| ACTGTGGATTGGCGCAGAGTCTCACGCCTTCAGGAAGATCCTGA | | |
| GGGTACGGAAAATCCGCTCGGTGTGGACGAGTCACTGTTCTCCT | | |
| ATGGCTTGAGGGAATCTATAGCGTCTTACCTTTCCTTGACGTCT | | |
| GAAGATAATACGTCTTTCGATCGCAAAAAAAAATCAATATCTCT | | |
| TATGTATGGCGGAAGCAAAAGGAAATCATCATTCTTTTCCTCTC | | |
| CACCATATTTCGAAGAT | | |
| CCAGCCCCAGCGGAGCGACCGCTGCCAAACCCTGAAGGGCTCG | 13 | SLAP1ₐₐ₈₋ₐₐ₂₄₇ |
| ACAGTGACTTTTTGGCTGTCCTCTCCGACTATCCTAGTCCCGATA | | intracellular signaling |
| TCAGCCCCCCGATATTCAGGCGCGGTGAAAAACTCCGAGTCATC | | domain |
| AGCGATGAGGGGGGTTGGTGGAAAGCCATCAGCCTGAGTACCG | | |
| GACGAGAGTCATACATTCCTGGAATATGTGTAGCGAGGGTGTA | | |
| CCACGGTTGGCTGTTCGAGGGTCTGGGAAGAGATAAGGCCGAG | | |
| GAACTTCTCCAACTCCCAGATACAAAAGTCGGTAGTTTTATGAT | | |
| TCGGGAAAGTGAAACTAAAAAGGGGTTCTATAGCCTCTCAGTT | | |
| CGGCATAGGCAGGTCAAGCATTATCGGATATTTCGCTTGCCTAA | | |
| CAACTGGTACTACATAAGTCCCCGACTCACATTCCAATGCCTGG | | |
| AGGACCTCGTGAATCACTATTCAGAAGTTGCAGATGGCCTCTGC | | |
| TGCGTACTCACGACACCCTGCCTTACCCAGAGTACAGCGGCCCC | | |
| GGCTGTTCGGGCATCTTCCAGCCCAGTAACACTCAGGCAAAAA | | |
| ACTGTGGATTGGCGCAGAGTCTCACGCCTTCAGGAAGATCCTGA | | |
| GGGTACGGAAAATCCGCTCGGTGTGGACGAGTCACTGTTCTCCT | | |
| ATGGCTTGAGGGAATCTATAGCGTCTTACCTTTCCTTGACGTCT | | |
| GAAGATAATACGTCTTTC | | |
| AGAAAATCCCTCCCCAGTCCAAGCCTGTCTAGTAGCGTTCAGGG | 14 | SLAP2ₐₐ₈₋ₐₐ₂₆₁ |
| TCAGGGCCCAGTTACTATGGAAGCGGAACGATCCAAGGCTACC | | intracellular signaling |
| GCAGTTGCTCTGGGTTCATTCCCGGCTGGAGGGCCAGCGGAACT | | domain |
| CTCCTTGCGCCTGGGTGAGCCACTTACCATCGTTTCTGAGGACG | | |
| GAGATTGGTGGACGGTTCTTTCTGAAGTATCTGGGAGAGAATAC | | |
| AACATACCGTCAGTTCATGTCGCAAAAGTTTCACACGGTTGGCT | | |
| TTACGAGGGACTCAGCAGGGAAAAAGCGGAGGAATTGTTGCTT | | |
| TTGCCCGGCAATCCTGGCGGAGCATTTTTGATAAGAGAGAGTCA | | |
| GACCCGGAGAGGCAGTTATTCCCTGTCAGTTAGACTCAGCAGA | | |
| CCTGCGAGTTGGGATAGGATTCGGCACTACAGGATTCACTGCCT | | |
| TGATAACGGCTGGCTGTATATATCTCCTCGCCTGACATTCCCTA | | |
| GCCTCCAAGCGTTGGTTGATCATTACTCTGAACTGGCAGACGAT | | |
| ATCTGCTGCCTGCTCAAGGAACCGTGCGTCCTCCAGAGGGCAG | | |
| GCCCACTTCCTGGCAAGGATATTCCACTTCCAGTCACGGTTCAA | | |
| AGAACCCCCCTCAATTGGAAGGAGCTGGATAGCTCTCTCCTCTT | | |
| TTCCGAGGCCGCTACAGGTGAAGAATCTCTGTTGTCTGAAGGAT | | |
| TGAGAGAGAGTCTCTCCTTCTACATCTCTCTGAATGATGAAGCA | | |
| GTGTCATTGGACGACGCA | | |
| AAAGTGAACGGCTGCCGGAAGTACAAGCTGAACAAGACCGAG | 15 | CD200R intracellular |
| AGCACCCCTGTGGTGGAAGAGGACGAGATGCAGCCTTACGCCA | | signaling domain |
| GCTACACCGAGAAGAACAACCCTCTGTACGACACCACCAACAA | | |
| AGTGAAGGCCAGCGAGGCCCTCCAGAGCGAGGTTGACACAGAT | | |
| CTGCACACCCTG | | |
| CACCGGTGGTGCAGCAACAAGAAAAACGCCGCCGTGATGGACC | 68 | KIR2DL1 intracellular |
| AAGAGAGCGCCGGAAATAGAACCGCCAACAGCGAGGATAGCG | | signaling domain |
| ACGAGCAGGACCCTCAAGAAGTGACCTACACACAGCTGAACCA | | |
| CTGCGTGTTCACCCAGCGGAAGATCACCAGACCTAGCCAGCGG | | |
| CCTAAGACACCACCTACCGACATCATCGTGTACACCGAGCTGCC | | |
| CAACGCCGAGAGCAGATCCAAGGTCGTGTCCTGTCCT | | |
| ATGACCGACAGCGTGATCTACAGCATGCTCGAGCTGCCTACAG | 69 | KLRG1 intracellular |
| CCACACAGGCCCAGAATGATTACGGCCCTCAGCAGAAGTCCAG | | signaling domain |
| CTCCAGCAGACCTAGCTGTAGCTGTCTTGGCTCCGGC | | |
| CACCGGCAGAACCAGATCAAGCAGGGCCCTCCTAGAAGCAAGG | 70 | LAIR1 intracellular |
| ACGAGGAACAGAAGCCTCAGCAGAGGCCTGATCTGGCCGTGGA | | signaling domain |
| CGTGCTGGAAAGAACAGCCGATAAGGCCACCGTGAACGGCCTG | | |
| CCTGAGAAGGACAGAGAGACAGACACATCTGCCCTGGCCGCTG | | |
| GCAGCTCCCAAGAAGTGACATACGCCCAGCTGGACCACTGGGC | | |
| CCTGACACAGAGAACTGCCAGAGCTGTGTCCCCTCAGAGCACC | | |
| AAACCTATGGCCGAGAGCATCACCTATGCCGCCGTGGCCAGACAT | | |
| CTGCGGCACAGAAGGCAGGGCAAGCACTGGACAAGCACCCAG | 71 | LIR2 intracellular |
| AGAAAGGCCGATTTTCAGCACCCTGCTGGCGCCGTTGGACCTGA | | signaling domain |
| GCCTACAGATAGAGGACTGCAGTGGCGGTCTAGCCCTGCTGCC | | |
| GATGCTCAAGAGGAAAACCTGTACGCCGCCGTGAAGGACACCC | | |
| AACCTGAAGATGGCGTGGAAATGGACACCAGAGCCGCCGCATC | | |
| TGAAGCCCCTCAGGATGTGACATACGCCCAGCTGCATAGCCTG | | |
| ACACTGAGGCGGAAAGCCACAGAGCCTCCACCTAGCCAAGAGA | | |
| GAGAGCCTCCTGCCGAGCCTAGCATCTATGCCACACTGGCCATT | | |
| CAC | | |
| CGGAGACAGAGACACAGCAAGCACAGAACCAGCGACCAGAGA | 72 | LIR3 intracellular |
| AAGACCGACTTCCAGAGGCCTGCTGGCGCCGCTGAGACAGAGC | | signaling domain |
| CTAAAGATAGAGGACTGCTGCGGAGAAGCAGCCCTGCTGCCGA | | |
| TGTGCAAGAGGAAAATCTGTACGCCGCCGTGAAGGACACCCAG | | |
| AGCGAGGATAGAGTGGAACTGGACAGCCAGTCTCCTCACGACG | | |
| AAGATCCTCAGGCCGTGACATACGCCCCTGTGAAGCACAGCAG | | |
| CCCTCGGAGAGAAATGGCCTCTCCACCTTCTAGCCTGAGCGGCG | | |
| AGTTCCTGGACACCAAGGACAGACAGGTGGAAGAGGACCGGCA | | |
| GATGGATACAGAAGCTGCCGCCTCTGAAGCCAGCCAGGATGTG | | |
| ACATATGCCCAGCTGCATAGCCTGACACTGCGGCGGAAAGCTA | | |
| CAGAGCCTCCTCCATCTCAAGAGGGCGAGCCTCCAGCCGAGCC | | |
| TAGCATCTATGCCACACTGGCCATTCAC | | |
| CAGCATTGGAGACAGGGAAAGCACAGAACACTGGCCCAGCGGC | 73 | LIR5 intracellular |
| AGGCCGATTTCCAAAGACCTCCAGGTGCCGCCGAACCTGAGCC | | signaling domain |
| TAAAGATGGTGGCCTGCAGCGGAGATCTTCTCCTGCTGCCGATG | | |
| TGCAGGGCGAGAATTTTTGTGCCGCCGTGAAGAACACCCAGCC | | |
| TGAGGATGGCGTGGAAATGGACACCAGACAGAGCCCTCACGAC | | |
| GAGGATCCACAGGCCGTGACATACGCCAAAGTGAAGCACAGCC | | |
| GGCCTCGGAGAGAAATGGCTAGCCCTCCAAGTCCTCTGAGCGG | | |
| CGAGTTCCTGGACACCAAAGACAGACAGGCCGAAGAGGACCGG | | |
| CAGATGGATACAGAAGCTGCCGCCTCTGAAGCCCCTCAGGATG | | |
| TGACATATGCCCAGCTGCATAGCTTCACCCTGCGGCAGAAAGCC | | |
| ACAGAGCCTCCACCTTCTCAAGAGGGCGCTTCTCCAGCCGAGCC | | |
| ATCTGTGTATGCCACACTGGCCATTCAC | | |
| AAGCTGCAGCGGAGATGGAAGAGAACACAGAGCCAGCAGGGC | 74 | SIGLEC-2 intracellular |
| CTGCAAGAGAATAGCAGCGGCCAGAGCTTCTTCGTGCGGAACA | | signaling domain |
| AGAAAGTGCGGAGAGCCCCTCTGTCTGAGGGACCTCATAGCCT | | |
| GGGCTGTTACAACCCCATGATGGAAGATGGCATCAGCTACACC | | |
| ACACTGCGGTTCCCCGAGATGAACATCCCTAGAACAGGCGACG | | |
| CCGAGAGCAGCGAAATGCAAAGACCTCCTCCTGACTGCGACGA | | |
| CACCGTGACATATAGCGCCCTGCACAAGAGACAAGTGGGCGAC | | |
| TACGAGAACGTGATCCCTGACTTCCCTGAGGACGAGGGCATCC | | |
| ACTACAGCGAGCTGATCCAGTTTGGCGTGGGCGAAAGACCCCA | | |
| GGCTCAAGAGAATGTGGACTACGTGATCCTGAAGCAC | | |
| AAGATCCTGCCTAAGCGGCGCACCCAGACCGAGACTCCTAGAC | 75 | SIGLEC-10 intracellular |
| CTAGATTCAGCCGGCACAGCACCATCCTGGACTACATCAACGTG | | signaling domain |
| GTGCCTACCGCCGGACCACTGGCTCAGAAGAGAAACCAGAAGG | | |
| CCACACCTAACAGCCCCAGAACACCTCTTCCACCTGGCGCACCT | | |
| TCTCCAGAGAGCAAGAAGAACCAGAAGAAGCAGTACCAGCTGC | | |
| CTAGCTTCCCCGAGCCTAAGAGCAGTACACAGGCCCCTGAGAG | | |
| CCAAGAGTCCCAAGAGGAACTGCACTACGCCACACTGAACTTC | | |
| CCCGGCGTCAGACCTAGACCTGAGGCCAGAATGCCTAAGGGCA | | |
| CCCAGGCCGATTACGCCGAAGTGAAGTTTCAA | | |

In some embodiments, one of the one or more intracellular signaling domains is derived from a protein selected from the group consisting of: SLAP1, SLAP2, Dok-1, Dok-2, LAIR1, GRB-2, CD200R, SIRPα, HAVR, GITR, PD-L1, KIR2DL1, KIR2DL2, KIR2DL3, KIR3DL2, CD94, KLRG-1, CEACAM1, LIR2, LIR3, LIR5, SIGLEC-2, and SIGLEC-10.

In some embodiments, the transmembrane domain is derived from the same protein as one of the one or more intracellular signaling domains. In some embodiments, the transmembrane domain is derived from a first protein and one of the one or more the intracellular signaling domains is derived from a second protein that is distinct from the first protein.

In some embodiments, one of the one or more intracellular signaling domains is derived from SLAP1.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 4)
PAPAERPLPNPEGLDSDFLAVLSDYPSPDISPPIFRRGEKLRVISDEG
GWWKAISLSTGRESYIPGICVARVYHGWLFEGLGRDKAEELLQLPDTK
VGSFMIRESETKKGFYSLSVRHRQVKHYRIFRLPNNWYYISPRLTFQC
LEDLVNHYSEVADGLCCVLTTPCLTQSTAAPAVRASSSPVTLRQKTVD
WRRVSRLQEDPEGTENPLGVDESLFSYGLRESIASYLSLTSEDNTSFD
RKKKSISLMYGGSKRKSSFFSSPPYFED,
or
(SEQ ID NO: 5)
PAPAERPLPNPEGLDSDFLAVLSDYPSPDISPPIFRRGEKLRVISDEG
GWWKAISLSTGRESYIPGICVARVYHGWLFEGLGRDKAEELLQLPDTK
VGSFMIRESETKKGFYSLSVRHRQVKHYRIFRLPNNWYYISPRLTFQC
LEDLVNHYSEVADGLCCVLTTPCLTQSTAAPAVRASSSPVTLRQKTVD
WRRVSRLQEDPEGTENPLGVDESLFSYGLRESIASYLSLTSEDNTSF.

In some embodiments, one of the one or more intracellular signaling domain comprises the amino acid sequence of

(SEQ ID NO: 4)
PAPAERPLPNPEGLDSDFLAVLSDYPSPDISPPIFRRGEKLRVISDEG
GWWKAISLSTGRESYIPGICVARVYHGWLFEGLGRDKAEELLQLPDTK
VGSFMIRESETKKGFYSLSVRHRQVKHYRIFRLPNNWYYISPRLTFQC
LEDLVNHYSEVADGLCCVLTTPCLTQSTAAPAVRASSSPVTLRQKTVD
WRRVSRLQEDPEGTENPLGVDESLFSYGLRESIASYLSLTSEDNTSFD
RKKKSISLMYGGSKRKSSFFSSPPYFED,
or
(SEQ ID NO: 5)
PAPAERPLPNPEGLDSDFLAVLSDYPSPDISPPIFRRGEKLRVISDEG
GWWKAISLSTGRESYIPGICVARVYHGWLFEGLGRDKAEELLQLPDTK
VGSFMIRESETKKGFYSLSVRHRQVKHYRIFRLPNNWYYISPRLTFQC
LEDLVNHYSEVADGLCCVLTTPCLTQSTAAPAVRASSSPVTLRQKTVD
WRRVSRLQEDPEGTENPLGVDESLFSYGLRESIASYLSLTSEDNTSF.

In some embodiments, one of the one or more intracellular signaling domain is derived from SLAP2.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 6)
RKSLPSPSLSSSVQGQGPVTMEAERSKATAVALGSFPAGGPAELSLRL
GEPLTIVSEDGDWWTVLSEVSGREYNIPSVHVAKVSHGWLYEGLSREK
AEELLLLPGNPGGAFLIRESQTRRGSYSLSVRLSRPASWDRIRHYRIH
CLDNGWLYISPRLTFPSLQALVDHYSELADDICCLLKEPCVLQRAGPL
PGKDIPLPVTVQRTPLNWKELDSSLLFSEAATGEESLLSEGLRESLSF
YISLNDEAVSLDDA.

In some embodiments, one of the one or more intracellular signaling domain comprises the amino acid sequence of

(SEQ ID NO: 6)
RKSLPSPSLSSSVQGQGPVTMEAERSKATAVALGSFPAGGPAELSLRL
GEPLTIVSEDGDWWTVLSEVSGREYNIPSVHVAKVSHGWLYEGLSREK
AEELLLLPGNPGGAFLIRESQTRRGSYSLSVRLSRPASWDRIRHYRIH
CLDNGWLYISPRLTFPSLQALVDHYSELADDICCLLKEPCVLQRAGPL
PGKDIPLPVTVQRTPLNWKELDSSLLFSEAATGEESLLSEGLRESLSF
YISLNDEAVSLDDA.

In some embodiments, one of the one or more intracellular signaling domain is derived from KIR2DL1.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 60)
HRWCSNKKNAAVMDQESAGNRTANSEDSDEQDPQEVTYTQLNHCVFTQ
RKITRPSQRPKTPPTDIIVYTELPNAESRSKVVSCP.

In some embodiments, one of the one or more intracellular signaling domain comprises the amino acid sequence of

(SEQ ID NO: 60)
HRWCSNKKNAAVMDQESAGNRTANSEDSDEQDPQEVTYTQLNHCVFTQ
RKITRPSQRPKTPPTDIIVYTELPNAESRSKVVSCP.

In some embodiments, one of the one or more intracellular signaling domain is derived from KLRG-1.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 61)
MTDSVIYSMLELPTATQAQNDYGPQQKSSSSRPSCSCLGSG.

In some embodiments, one of the one or more intracellular signaling domain comprises the amino acid sequence of

(SEQ ID NO: 61)
MTDSVIYSMLELPTATQAQNDYGPQQKSSSSRPSCSCLGSG.

In some embodiments, one of the one or more intracellular signaling domain is derived from LAIR1.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 62)
HRQNQIKQGPPRSKDEEQKPQQRPDLAVDVLERTAD
KATVNGLPEKDRETDTSALAAGSSQEVTYAQLDHWA
LTQRTARAVSPQSTKPMAESITYAAVARH.

In some embodiments, one of the one or more intracellular signaling domain comprises the amino acid sequence of

(SEQ ID NO: 62)
HRQNQIKQGPPRSKDEEQKPQQRPDLAVDVLERTAD
KATVNGLPEKDRETDTSALAAGSSQEVTYAQLDHWA
LTQRTARAVSPQSTKPMAESITYAAVARH.

In some embodiments, one of the one or more intracellular signaling domain is derived from LIR2.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 63)
LRHRRQGKHWTSTQRKADFQHPAGAVGPEPTDRGLQ
WRSSPAADAQEENLYAAVKDTQPEDGVEMDTRAAAS
EAPQDVTYAQLHSLTLRRKATEPPPSQEREPPAEPS
IYATLAIH.

In some embodiments, one of the one or more intracellular signaling domain comprises the amino acid sequence of

(SEQ ID NO: 63)
LRHRRQGKHWTSTQRKADFQHPAGAVGPEPTDRGLQ
WRSSPAADAQEENLYAAVKDTQPEDGVEMDTRAAAS
EAPQDVTYAQLHSLTLRRKATEPPPSQEREPPAEPS
IYATLAIH.

In some embodiments, one of the one or more intracellular signaling domain is derived from LIR3.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 64)
RRQRHSKHRTSDQRKTDFQRPAGAAETEPKDRGLLR
RSSPAADVQEENLYAAVKDTQSEDRVELDSQSPHDE
DPQAVTYAPVKHSSPRREMASPPSSLSGEFLDTKDR
QVEEDRQMDTEAAASEASQDVTYAQLHSLTLRRKAT
EPPPSQEGEPPAEPSIYATLAIH.

In some embodiments, one of the one or more intracellular signaling domain comprises the amino acid sequence of

(SEQ ID NO: 64)
RRQRHSKHRTSDQRKTDFQRPAGAAETEPKDRGLLR
RSSPAADVQEENLYAAVKDTQSEDRVELDSQSPHDE
DPQAVTYAPVKHSSPRREMASPPSSLSGEFLDTKDR
QVEEDRQMDTEAAASEASQDVTYAQLHSLTLRRKAT
EPPPSQEGEPPAEPSIYATLAIH.

In some embodiments, one of the one or more intracellular signaling domain is derived from LIR5.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 65)
QHWRQGKHRTLAQRQADFQRPPGAAEPEPKDGGLQR
RSSPAADVQGENFCAAVKNTQPEDGVEMDTRQSPHD
EDPQAVTYAKVKHSRPRREMASPPSPLSGEFLDTKD
RQAEEDRQMDTEAAASEAPQDVTYAQLHSFTLRQKA
TEPPPSQEGASPAEPSVYATLAIH.

In some embodiments, one of the one or more intracellular signaling domain comprises the amino acid sequence of

(SEQ ID NO: 65)
QHWRQGKHRTLAQRQADFQRPPGAAEPEPKDGGLQR
RSSPAADVQGENFCAAVKNTQPEDGVEMDTRQSPHD
EDPQAVTYAKVKHSRPRREMASPPSPLSGEFLDTKD
RQAEEDRQMDTEAAASEAPQDVTYAQLHSFTLRQKA
TEPPPSQEGASPAEPSVYATLAIH.

In some embodiments, one of the one or more intracellular signaling domain is derived from SIGLEC-2.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 66)
KLQRRWKRTQSQQGLQENSSGQSFFVRNKKVRRAPL
SEGPHSLGCYNPMMEDGISYTTLRFPEMNIPRTGDA
ESSEMQRPPPDCDDTVTYSALHKRQVGDYENVIPDF
PEDEGIHYSELIQFGVGERPQAQENVDYVILKH.

In some embodiments, one of the one or more intracellular signaling domain comprises the amino acid sequence of

(SEQ ID NO: 66)
KLQRRWKRTQSQQGLQENSSGQSFFVRNKKVRRAPL
SEGPHSLGCYNPMMEDGISYTTLRFPEMNIPRTGDA
ESSEMQRPPPDCDDTVTYSALHKRQVGDYENVIPDF
PEDEGIHYSELIQFGVGERPQAQENVDYVILKH.

In some embodiments, one of the one or more intracellular signaling domain is derived from SIGLEC-10.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 67)
KILPKRRTQTETPRPRFSRHSTILDYINVVPTAGPL
AQKRNQKATPNSPRTPLPPGAPSPESKKNQKKQYQL
PSFPEPKSSTQAPESQESQEELHYATLNFPGVRPRP
EARMPKGTQADYAEVKFQ.

In some embodiments, one of the one or more intracellular signaling domain comprises the amino acid sequence of

(SEQ ID NO: 67)
KILPKRRTQTETPRPRFSRHSTILDYINVVPTAGPL
AQKRNQKATPNSPRTPLPPGAPSPESKKNQKKQYQL
PSFPEPKSSTQAPESQESQEELHYATLNFPGVRPRP
EARMPKGTQADYAEVKFQ.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 1. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 2. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 3. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 4. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 7. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 8.

In some embodiments, the transmembrane domain and one of the one or more intracellular signaling domain are derived from the same protein. In some embodiments, the transmembrane domain is derived from a first protein and one of the one or more intracellular signaling domain is derived from a second protein that is distinct from the first protein.

Enzymatic Inhibitory Domains

In some embodiments, the inhibitory chimeric receptor comprises an enzymatic inhibitory domain. In some embodiments, the enzymatic inhibitory domain is also capable of preventing, attenuating, or inhibiting activation of a chimeric receptor when expressed on an immunomodulatory cell relative to an otherwise identical chimeric inhibitory receptor lacking the enzymatic inhibitory domain.

In some embodiments, the enzymatic inhibitory domain comprises an enzyme catalytic domain. In some embodiments, the enzyme catalytic domain is derived from an enzyme selected from the group consisting of: CSK, SHP-1, PTEN, CD45, CD148, PTP-MEG1, PTP-PEST, c-CBL, CBL-b, PTPN22, LAR, PTPH1, SHIP-1, and RasGAP.

In some embodiments, the enzymatic inhibitory domain comprises one or more modifications that modulate basal prevention, attenuation, or inhibition relative to an otherwise identical enzymatic inhibitory domain lacking the one or more modifications. In some embodiments, the one or more modifications reduce basal prevention, attenuation, or inhibition relative to an otherwise identical enzymatic inhibitory domain lacking the one or more modifications. In some embodiments, the one or more modifications increase basal prevention, attenuation, or inhibition relative to an otherwise identical enzymatic inhibitory domain lacking the one or more modifications.

Activation and Co-Stimulatory Domains

In some embodiments, a cell disclosed herein can further comprise at least one tumor-targeting chimeric receptor or T cell receptor comprising an activating intracellular domain or a co-stimulatory intracellular domain. In some embodiments, the cell comprises at least one inhibitory chimeric receptor and at least one tumor-targeting chimeric receptor. The cell can comprise at least 1, at least 2, at least 3, at least 4, at least 5, at least 6, at least 7, at least 8, at least 9, or at least 10 or more tumor-targeting CARs and at least 1, at least 2, at least 3, at least 4, at least 5, at least 6, at least 7, at least 8, at least 9, or at least 10 or more inhibitory chimeric receptors.

In some embodiments, the activating signaling domain is a CD3-zeta protein, which includes three immunoreceptor tyrosine-based activation motifs (ITAMs). Other examples of activating signaling domains include CD28, 4-1BB, and OX40. In some embodiments, a cell receptor comprises more than one activating signaling domain, each referred to as a co-stimulatory domain.

In some embodiments, the tumor-targeting chimeric receptor is a chimeric antigen receptor (CAR) or an engineered T cell receptor. In some embodiments, the CAR binds one or more proteins expressed on the surface of a tumor cell.

In some embodiments, prior to binding of the protein to the chimeric inhibitory receptor, the tumor-targeting chimeric receptor is capable of activating the cell.

Transmembrane Domains

The inhibitory chimeric receptors can contain transmembrane domains that link the protein binding domain to the intracellular domain. Different transmembrane domains result in different receptor stability. Suitable transmembrane domains include, but are not limited to, CD8, CD28, CD3zeta, CD4, 4-IBB, OX40, ICOS, 2B4, CD25, CD7, LAX, LAT, LAIR1, GRB-2, Dok-1, Dok-2, SLAP1, SLAP2, CD200R, SIRPalpha, HAVR, GITR, PD-L1, KIR2DL1, KIR2DL2, KIR2DL3, KIR3DL2, CD94, KLRG-1, CEACAM1, LIR2, LIR3, LIR5, SIGLEC-2, and SIGLEC-10.

In some embodiments, the transmembrane domain is derived from a protein selected from the group consisting of: CD8, CD28, CD3zeta, CD4, 4-IBB, OX40, ICOS, 2B4, CD25, CD7, LAX, LAT, LAIR1, GRB-2, Dok-1, Dok-2, SLAP1, SLAP2, CD200R, SIRPalpha, HAVR, GITR, PD-L1, KIR2DL1, KIR2DL2, KIR2DL3, KIR3DL2, CD94, KLRG-1, CEACAM1, LIR2, LIR3, LIR5, SIGLEC-2, and SIGLEC-10. In some embodiments, a transmembrane domain of a cell receptor is an LAX transmembrane domain. In some embodiments, a transmembrane domain of a cell receptor is a CD28 transmembrane domain. In some embodiments, a transmembrane domain of a cell receptor is a CD25 transmembrane domain. In some embodiments, a transmembrane domain of a cell receptor is a CD7 transmembrane domain. In some embodiments, a transmembrane domain of a cell receptor is an LAT transmembrane domain. In some embodiments, a transmembrane domain of a cell receptor is a SIRPα transmembrane domain.

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from CD28.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to FWVLVVVGGVLACYSLLVTVAFIIFWV (SEQ ID NO:20). In some embodiments, the transmembrane domain comprises the amino acid sequence of FWVLVVVGGVLACYSLLVTVAFIIFWV (SEQ ID NO:20).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from KIR2DL1.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to ILIGTSVVIILFILLFFLL (SEQ ID NO:76). In some embodiments, the transmembrane domain comprises the amino acid sequence of ILIGTSVVIILFILLFFLL (SEQ ID NO:76).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from KLRG-1.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VAIALGLLTAVLLSVLLYQWI (SEQ ID NO: 78). In some embodiments, the transmembrane domain comprises the amino acid sequence of

(SEQ ID NO: 78)
VAIALGLLTAVLLSVLLYQWI.

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from LAIR1.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to ILIGVSVVFLFCLLLLVLFCL (SEQ ID NO: 79). In some embodiments, the transmembrane domain comprises the amino acid sequence of

(SEQ ID NO: 79)
ILIGVSVVFLFCLLLLVLFCL.

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from LIR2.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VIGILVAVVLLLLLLLLLFLI (SEQ ID NO: 80). In some embodiments, the transmembrane domain comprises the amino acid sequence of

(SEQ ID NO: 80)
VIGILVAVVLLLLLLLLLFLI.

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from LIR3.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VLIGVSVAFVLLLFLLLFLLL (SEQ ID NO: 81). In some embodiments, the transmembrane domain comprises the amino acid sequence of

(SEQ ID NO: 81)
VLIGVSVAFVLLLFLLLFLLL.

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from LIR5.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VLIGVLVVSILLLSLLLFLLL (SEQ ID NO: 82). In some embodiments, the transmembrane domain comprises the amino acid sequence of

(SEQ ID NO: 82)
VLIGVLVVSILLLSLLLFLLL.

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from SIGLEC-2.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VAVGLGSCLAILILAICGL (SEQ ID NO: 83). In some embodiments, the transmembrane domain comprises the amino acid sequence of VAVGLGSCLAILILAICGL (SEQ ID NO: 83).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from SIGLEC-10.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to GAFLGIGITALLFLCLALIIM (SEQ ID NO: 84). In some embodiments, the transmembrane domain comprises the amino acid sequence of

(SEQ ID NO: 84)
GAFLGIGITALLFLCLALIIM.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 16. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO:17. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO:18. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO:19. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO:21.

Exemplary transmembrane domain amino acid sequences are shown in Table 3. Exemplary transmembrane domain nucleic acid sequences are shown in Table 4.

TABLE 3
Exemplary transmembrane domain
amino acid sequences

| | SEQ | |
|---|---|---|
| Amino Acid | ID | |
| Sequence | NO: | Description |
| IFSGFAGLLA | 16 | LAX transmembrane domain |
| ILLVVAVFCI | | |
| L | | |
| VAVAGCVFLL | 17 | CD25 transmembrane domain |
| ISVLLLSGL | | |
| AALAVISFLL | 18 | CD7 transmembrane domain |
| GLGLGVACVL | | |
| A | | |
| MEADALSPVG | 19 | LAT transmembrane domain |
| LGLLLLPFLV | | |
| TLLAALAVRA | | |
| RELPVS | | |
| FWVLVVVGGV | 20 | CD28 transmembrane domain |
| LACYSLLVTV | | |
| AFIIFWV | | |
| IVVGVVCTLL | 21 | SIRPalpha transmembrane |
| VALLMAALYL | | domain |
| V | | |
| ILIGTSVVII | 76 | KIR2DL1 transmembrane domain |
| LFILLFFLL | | |
| VIGILVAVIL | 77 | LIR1 transmembrane domain |
| LLLLLLLLFL | | |
| I | | |
| VAIALGLLTA | 78 | KLRG1 transmembrane domain |
| VLLSVLLYQW | | |
| I | | |
| ILIGVSVVFL | 79 | LAIR1 transmembrane domain |
| FCLLLLVLFC | | |
| L | | |
| VIGILVAVVL | 80 | LIR2 transmembrane domain |
| LLLLLLLLFL | | |
| I | | |
| VLIGVSVAFV | 81 | LIR3 transmembrane domain |
| LLLFLLLFLL | | |
| L | | |
| VLIGVLVVSI | 82 | LIR5 transmembrane domain |
| LLLSLLLFLL | | |
| L | | |
| VAVGLGSCLA | 83 | SIGLEC-2 transmembrane |
| ILILAICGL | | domain |
| GAFLGIGITA | 84 | SIGLEC-10 transmembrane |
| LLFLCLALII | | domain |
| M | | |

Exemplary transmembrane domain nucleic
acid sequences

| | SEQ | |
|---|---|---|
| Nucleic Acid | ID | |
| Sequence | NO: | Description |
| TTTTGGGTGCTGGTGGTG | 22 | CD28 transmembrane |
| GTTGGTGGAGTCCTGGC | | domain |
| TTGCTATAGCTTGCTAGT | | |
| AACAGTGGCCTTTATTAT | | |
| TTTCTGGGTG | | |
| ATCCTGATCGGCACCAGC | 85 | KIR2DL1 transmembrane |
| GTGGTCATCATCCTGTT | | domain |
| TATCCTGCTGTTCTTCCT | | |
| GCTG | | |
| GTGATCGGCATTCTGGTG | 86 | LIR1 transmembrane |
| GCCGTGATTCTGCTGCT | | domain |
| CCTGCTGTTGCTGCTGCT | | |
| GTTCCTGATC | | |
| GTGGCCATTGCTCTGGGA | 87 | KLRG1 transmembrane |
| CTGCTTACAGCCGTGCT | | domain |
| GCTGAGTGTGCTGCTGTA | | |
| CCAGTGGATC | | |
| ATCCTGATCGGAGTGTCC | 88 | LAIR1 transmembrane |
| GTGGTGTTTCTGTTCTGC | | domain |
| CTGCTCCTGCTGGTGCTG | | |
| TTCTGTCTG | | |
| GTGATCGGAATTCTGGTG | 89 | LIR2 transmembrane |
| GCCGTGGTGCTGCTCCT | | domain |
| GCTGCTTCTCCTTCTGCT | | |
| GTTCCTGATC | | |
| GTGCTGATCGGAGTGTCT | 90 | LIR3 transmembrane |
| GTGGCTTTCGTGCTGCT | | domain |
| CCTGTTCCTCCTGCTGTT | | |
| CCTGCTCCTG | | |
| GTGCTGATTGGCGTGCTG | 91 | LIR5 transmembrane |
| GTGGTGTCTATCCTGCT | | domain |
| CCTGTCACTGCTGCTGTT | | |
| TCTGCTGCTC | | |
| GTGGCCGTTGGCCTGGGA | 92 | SIGLEC-2 |
| TCTTGTCTGGCCATTCT | | transmembrane |
| GATCCTGGCCATCTGCGG | | domain |
| CCTG | | |
| GGCGCCTTTCTCGGCATC | 93 | SIGLEC-10 |
| GGAATTACAGCCCTGCT | | transmembrane |
| GTTCCTGTGCCTGGCTCT | | domain |
| GATCATCATG | | |

In some embodiments, the transmembrane domain is physically linked to the extracellular protein binding domain. In some embodiments, the intracellular signaling domain is physically linked to the transmembrane domain. In some embodiments, the transmembrane domain is physically linked to the extracellular protein binding domain and the intracellular signaling domain is physically linked to the transmembrane domain.

In some embodiments, the one or more intracellular signaling domains are two intracellular signaling domains.

In some embodiments, the chimeric inhibitory receptor comprises a first intracellular signaling domain derived from KIR2DL1 and a second intracellular signaling domain derived from LIR2. In some embodiments, the chimeric inhibitory receptor comprises a first intracellular signaling domain derived from KIR2DL1 and a second intracellular signaling domain derived from LIR3. In some embodiments, the chimeric inhibitory receptor comprises a first intracellular signaling domain derived from KIR2DL1 and a second intracellular signaling domain derived from LIR5. In some embodiments, the first intracellular signaling domain further comprises a transmembrane domain derived from KIR2DL1.

In some embodiments, the chimeric inhibitory receptor comprises a first intracellular signaling domain derived from LIR2 and a second intracellular signaling domain derived from KIR2DL1. In some embodiments, the first intracellular signaling domain further comprises a transmembrane domain derived from LIR2.

In some embodiments, the chimeric inhibitory receptor comprises a first intracellular signaling domain derived from LIR3 and a second intracellular signaling domain derived from KIR2DL1. In some embodiments, the first intracellular signaling domain further comprises a transmembrane domain derived from LIR3.

In some embodiments, the chimeric inhibitory receptor comprises a first intracellular signaling domain derived from LIR5 and a second intracellular signaling domain derived from KIR2DL1. In some embodiments, the first intracellular signaling domain further comprises a transmembrane domain derived from LIR5.

Extracellular Protein Binding Domains

The inhibitory chimeric receptors described herein further comprise extracellular protein binding domains.

In some embodiments, immune cells expressing an inhibitory chimeric receptor are genetically modified to recognize multiple targets or antigens, which permits the recognition of unique target or protein expression patterns on tumor cells.

In some embodiments, the protein is not expressed on the target tumor. In some embodiments, the expression in non-tumor cells is at least 2-fold, at least 3-fold, at least 4-fold, at least 5-fold, at least 6-fold, at least 7-fold, at least 8-fold, at least 9-fold, or at least 10-fold or more lower than the level of expression that would result in activation of the tumor-targeting chimeric antigen receptor.

In some embodiments, the protein is expressed on a non-tumor cell.

In some embodiments, the protein is expressed on a non-tumor cell derived from a tissue selected from the group consisting of brain, neuronal tissue, endocrine, endothelial, bone, bone marrow, immune system, muscle, lung, liver, gallbladder, pancreas, gastrointestinal tract, kidney, urinary bladder, male reproductive organs, female reproductive organs, adipose, soft tissue, and skin.

In some embodiments, the extracellular protein binding domain comprises a ligand-binding domain. In some embodiments, the ligand-binding domain can be a domain from a receptor, wherein the receptor is selected from the group consisting of a T cell receptor (TCR), a B cell receptor (BCR), a cytokine receptor, an RTK receptor, a serine/threonine kinase receptor, a hormone receptor, an immunoglobulin superfamily receptor, and a TNFR-superfamily receptor. In some embodiments, the extracellular protein binding domain comprises a receptor-binding domain. In some embodiments, the extracellular protein binding domain comprises an antigen-binding domain.

In some embodiments, an extracellular protein binding domain of a inhibitory chimeric receptor of the disclosure comprises an antigen binding domain, such as a single chain Fv (scFv) specific for a tumor antigen. In some embodiments, an extracellular protein binding domain comprises an antibody, an antigen-binding fragment thereof, F(ab), F(ab′), a single chain variable fragment (scFv), or a single-domain antibody (sdAb).

The term “single-chain” refers to a molecule comprising amino acid monomers linearly linked by peptide bonds. In a particular such embodiment, the C-terminus of the Fab light chain is connected to the N-terminus of the Fab heavy chain in the single-chain Fab molecule. As described in more detail herein, an scFv has a variable domain of light chain (VL) connected from its C-terminus to the N-terminal end of a variable domain of heavy chain (VH) by a polypeptide chain. Alternately the scFv comprises of polypeptide chain where in the C-terminal end of the VH is connected to the N-terminal end of VL by a polypeptide chain.

The “Fab fragment” (also referred to as fragment antigen-binding) contains the constant domain (CL) of the light chain and the first constant domain (CH1) of the heavy chain along with the variable domains VL and VH on the light and heavy chains respectively. The variable domains comprise the complementarity determining loops (CDR, also referred to as hypervariable region) that are involved in antigen-binding. Fab′ fragments differ from Fab fragments by the addition of a few residues at the carboxy terminus of the heavy chain CH1 domain including one or more cysteines from the antibody hinge region.

“F(ab′)2” fragments contain two Fab′ fragments joined, near the hinge region, by disulfide bonds. F(ab′)2 fragments may be generated, for example, by recombinant methods or by pepsin digestion of an intact antibody. The F(ab′) fragments can be dissociated, for example, by treatment with B-mercaptoethanol.

“Fv” fragments comprise a non-covalently-linked dimer of one heavy chain variable domain and one light chain variable domain.

“Single-chain Fv” or “sFv” or “scFv” includes the VH and VL domains of an antibody, wherein these domains are present in a single polypeptide chain. In one embodiment, the Fv polypeptide further comprises a polypeptide linker between the VH and VL domains which enables the scFv to form the desired structure for antigen-binding.

The term “single domain antibody” or “sdAb” refers to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety. Single domain antibodies are also known as sdAbs or nanobodies. Sdabs are fairly stable and easy to express as fusion partner with the Fc chain of an antibody (Harmsen M M, De Haard H J (2007). “Properties, production, and applications of camelid single-domain antibody fragments”. Appl. Microbiol Biotechnol. 77(1): 13-22).

An “antibody fragment” comprises a portion of an intact antibody, such as the antigen-binding or variable region of an intact antibody. Antibody fragments include, for example, Fv fragments, Fab fragments, F(ab′)2 fragments, Fab′ fragments, scFv (sFv) fragments, and scFv-Fc fragments.

In some embodiments, the antigen-binding domain comprises an antibody, an antigen-binding fragment of an antibody, a F(ab) fragment, a F(ab′) fragment, a single chain variable fragment (scFv), or a single-domain antibody (sdAb). In some embodiments, the antigen-binding domain comprises a single chain variable fragment (scFv). In some embodiments, each scFv comprises a heavy chain variable domain (VH) and a light chain variable domain (VL). In some embodiments, the VH and VL are separated by a peptide linker.

In some embodiments, the extracellular protein binding domain comprises a ligand-binding domain. The ligand-binding domain can be a domain from a receptor, wherein the receptor is selected from the group consisting of TCR, BCR, a cytokine receptor, RTK receptors, serine/threonine kinase receptors, hormone receptors, immunoglobulin superfamily receptors, and TNFR-superfamily of receptors. In some embodiments, an extracellular protein binding domain binds to a target protein comprising CD20 or CD19.

The choice of binding domain depends upon the type and number of ligands that define the surface of a target cell. For example, the extracellular protein binding domain may be chosen to recognize a ligand that acts as a cell surface marker on target cells associated with non-disease states, such as “self” or normal tissue, or the extracellular protein binding domain may be chosen to recognize a ligand that acts as a cell surface marker on targets associated with a particular disease state, such as cancer or an autoimmune disease. In general, an inhibitory chimeric receptor binding domain may be selected from a non-disease state cell surface marker, while a tumor-targeting chimeric receptor binding domain may be selected from a disease state cell surface marker. Thus, examples of cell surface markers that may act as ligands for the extracellular protein binding domain in the inhibitory chimeric receptor of the present disclosure include those associated with normal tissue and examples of cell surface markers that may act as ligands for the protein binding domain in a tumor-targeting chimeric receptor include those associated with cancer cells and/or other forms of diseased cells. In some embodiments, an inhibitory chimeric receptor is engineered to target a non-tumor protein of interest by way of engineering a desired protein binding domain that specifically binds to a protein on a non-tumor cell encoded by an engineered nucleic acid.

An extracellular protein binding domain (e.g., an scFv) that specifically binds to a target or an epitope is a term understood in the art, and methods to determine such specific binding are also known in the art. A molecule is said to exhibit specific binding if it reacts or associates more frequently, more rapidly, with greater duration and/or with greater affinity with a particular target protein than it does with alternative targets. An extracellular protein binding domain (e.g., an scFv) that specifically binds to a first target protein may or may not specifically bind to a second target protein. As such, specific binding does not necessarily require (although it can include) exclusive binding. In some embodiments, an extracellular protein binding domain is an antigen-binding domain.

In some embodiments, the extracellular protein binding domain has a high binding affinity.

In some embodiments, the extracellular protein binding domain has a low binding affinity.

Linkers

In some embodiments, the inhibitory chimeric receptor comprises a peptide linker. A linker is generally used to link two peptides of a protein binding domain, such as the peptides of an scFv or sdAb. Any appropriate linker known in the art may be used, including glycerin-serine based linkers. In some embodiments, the heavy chain variable domain (VH) and light chain variable domain (VL) of an scFv are separated by a peptide linker. In some embodiments, the scFv comprises the structure VH-L-VL or VL-L-VH, wherein VH is the heavy chain variable domain, L is the peptide linker, and VL is the light chain variable domain. In some embodiments, the peptide linker comprises an amino acid sequence selected from the group consisting of GGS (SEQ ID NO: 23), GGSGGS (SEQ ID NO: 24), GGSGGSGGS (SEQ ID NO: 25), GGSGGSGGSGGS (SEQ ID NO: 26), GGSGGSGGSGGSGGS (SEQ ID NO: 27), GGGS (SEQ ID NO: 28), GGGSGGGS (SEQ ID NO: 29), GGGSGGGSGGGS (SEQ ID NO: 30), GGGSGGGSGGGSGGGS (SEQ ID NO: 31), GGGSGGGSGGGSGGGSGGGS (SEQ ID NO: 32), GGGGS (SEQ ID NO: 33), GGGGSGGGGS (SEQ ID NO: 34), GGGGSGGGGSGGGGS (SEQ ID NO: 35), GGGGSGGGGSGGGGSGGGGS (SEQ ID NO: 36), GGGGSGGGGSGGGGSGGGGSGGGGS (SEQ ID NO: 37), and TTTPAPRPPTPAPTIALQPLSLRPEACRPAAGGAVHTRGLDFACDQTTPGERSSLPAFY PGTSGSCSGCGSLSLP (SEQ ID NO: 94).

Exemplary linker amino acid sequences are shown in Table 5. An exemplary linker nucleic acid sequence is shown in Table 6.

TABLE 5
Exemplary linker amino acid sequences

| | SEQ | |
|---|---|---|
| | ID | |
| Amino Acid Sequence | NO: | Description |
| GGS | 23 | (G₂S)₁ scFv linker |
| GGSGGS | 24 | (G₂S)₂ scFv linker |
| GGSGGSGGS | 25 | (G₂S)₃ scFv linker |
| GGSGGSGGSGGS | 26 | (G₂S)₄ scFv linker |
| GGSGGSGGSGGSGGS | 27 | (G₂S)₅ scFv linker |
| GGGS | 28 | (G₃S)₁ scFv linker |
| GGGSGGGS | 29 | (G₃S)₂ scFv linker |
| GGGSGGGSGGGS | 30 | (G₃S)₃ scFv linker |
| GGGSGGGSGGGSGGGS | 31 | (G₃S)₄ scFv linker |
| GGGSGGGSGGGSGGGSGGGS | 32 | (G₃S)₅ scFv linker |
| GGGGS | 33 | (G₄S)₁ scFv linker |
| GGGGSGGGGS | 34 | (G₄S)₂ scFv linker |
| GGGGSGGGGSGGGGS | 35 | (G₄S)₃ scFv linker |
| GGGGSGGGGSGGGGSGGGGS | 36 | (G₄S)₄ scFv linker |
| GGGGSGGGGSGGGGSGGGGS | 37 | (G₄S)₅ scFv linker |
| GGGGS | | |
| TTTPAPRPPTPAPTIALQPL | 94 | linker |
| SLRPEACRPAAGGAVHTRG | | |
| LDFACDQTTPGERSSLPAF | | |
| YPGTSGSCSGCGSLSLP | | |

TABLE 6
Exemplary linker nucleic acid sequence

| | SEQ | |
|---|---|---|
| | ID | |
| Nucleic Acid Sequence | NO: | Description |
| GGAGGCGGAGGATCTGGTGGC | 38 | (G₄S)₃ scFv linker |
| GGAGGAAGTGGCGGAGGCGGTTCT | | |

Spacers or Hinge Domains

Chimer receptors can also contain spacer or hinge domains in the polypeptide. In some embodiments, a spacer domain or a hinge domain is located between an extracellular domain (e.g., comprising the protein binding domain) and a transmembrane domain of an inhibitory chimeric receptor or tumor-targeting chimeric receptor, or between a intracellular signaling domain and a transmembrane domain of the inhibitory chimeric receptor or tumor-targeting chimeric receptor. A spacer or hinge domain is any oligopeptide or polypeptide that functions to link the transmembrane domain to the extracellular domain and/or the intracellular signaling domain in the polypeptide chain. Spacer or hinge domains provide flexibility to the inhibitory chimeric receptor or tumor-targeting chimeric receptor, or domains thereof, or prevent steric hindrance of the inhibitory chimeric receptor or tumor-targeting chimeric receptor, or domains thereof. In some embodiments, a spacer domain or hinge domain may comprise up to 300 amino acids (e.g., 10 to 100 amino acids, or 5 to 20 amino acids). In some embodiments, one or more spacer domain(s) may be included in other regions of an inhibitory chimeric receptor or tumor-targeting chimeric receptor.

Exemplary spacer or hinge domain amino acid sequences are shown in Table 7. Exemplary spacer or hinge domain nucleic acid sequences are shown in Table 8.

TABLE 7
Exemplary spacer or hinge domain
amino acid sequences

| | SEQ | |
|---|---|---|
| Amino Acid | ID | |
| Sequence | NO: | Description |
| AAAIEVMYPPPYLDNEKS | 39 | CD28 hinge |
| NGTIIHVKGKHLCPSPLF | | |
| PGPSKP | | |
| ESKYGPPCPSCP | 40 | IgG4 minimal |
| | | hinge |
| ESKYGPPAPSAP | 41 | IgG4 minimal |
| | | hinge, |
| | | no disulfides |
| ESKYGPPCPPCP | 42 | IgG4 S228P |
| | | minimal hinge, |
| | | enhanced |
| | | disulfide |
| | | formation |
| EPKSCDKTHTCP | 43 | IgG1 minimal |
| | | hinge |
| AAAFVPVFLPAKPTTTPAP | 44 | Extended |
| RPPTPAPTIASQPLSLRPE | | CD8a hinge |
| ACRPAAGGAVHTRGLDFAC | | |
| DIYIWAPLAGTCGVLLLSL | | |
| VITLYCNHRN | | |
| TTTPAPRPPTPAPTIALQP | 45 | CD8a hinge |
| LSLRPEACRPAAGGAVHTR | | |
| GLDFACD | | |
| ACPTGLYTHSGECCKACNL | 46 | LNGFR hinge |
| GEGVAQPCGANQTVCEPCL | | |
| DSVTFSDVVSATEPCKPCT | | |
| ECVGLQSMSAPCVEADDAV | | |
| CRCAYGYYQDETTGRCEAC | | |
| RVCEAGSGLVFSCQDKQNT | | |
| VCEECPDGTYSDEADAEC | | |
| ACPTGLYTHSGECCKACNL | 47 | Truncated LNGFR |
| GEGVAQPCGANQTVC | | hinge (TNFR- |
| | | Cys1) |
| AVGQDTQEVIVVPHSLPF | 48 | PDGFR-beta |
| KV | | extracellular |
| | | linker |

TABLE 8
Exemplary spacer or hinge domain nucleic acid sequences

| Nucleic Acid Sequence | SEQ ID NO: | Description |
|---|---|---|
| GCAGCAGCTATCGAGGTGATGTATCCTCCGCCCTA | 49 | CD28 hinge |
| CCTGGATAATGAAAAGAGTAATGGGACTATCATTC | | |
| ATGTAAAAGGGAAGCATCTTTGTCCTTCTCCCCTTT | | |
| TCCCCGGTCCGTCTAAACCT | | |
| GAA AGC AAG TAC GGT CCA CCT TGC CCT AGC | 50 | IgG4 minimal hinge |
| TGT CCG | | |
| GAA TCC AAG TAC GGC CCC CCA GCG CCT AGT | 51 | IgG4 minimal hinge, no |
| GCC CCA | | disulfides |
| GAA TCT AAA TAT GGC CCG CCA TGC CCG CCT | 52 | IgG4 S228P minimal hinge, |
| TGC CCA | | enhanced disulfide formation |
| GAA CCG AAG TCT TGT GAT AAA ACT CAT ACG | 53 | IgGI minimal hinge |
| TGC CCG | | |
| GCT GCT GCT TTC GTA CCC GTG TTC CTC CCT | 54 | Extended CD8a hinge |
| GCT AAG CCT ACG ACT ACC CCC GCA CCG AGA | | |
| CCA CCC ACG CCA GCA CCC ACG ATTGCT AGC | | |
| CAG CCC CTT AGT TTG CGA CCA GAA GCT TGT | | |
| CGG CCT GCT GCT GGT GGC GCG GTA CAT ACC | | |
| CGC GGC CTT GAT TTT GCTTGC GAT ATA TAT | | |
| ATC TGG GCG CCT CTG GCC GGA ACA TGC GGG | | |
| GTC CTC CTC CTT TCT CTG GTT ATT ACT CTC | | |
| TAC TGT AAT CACAGG AAT | | |
| GCC TGC CCG ACC GGG CTC TAC ACT CAT AGC | 55 | LNGFR hinge |
| GGG GAA TGT TGT AAG GCA TGT AAC TTG GGT | | |
| GAG GGC GTC GCA CAG CCC TGC GGAGCT AAC | | |
| CAA ACA GTG TGC GAA CCC TGC CTC GAT AGT | | |
| GTG ACG TTC TCT GAT GTT GTA TCA GCT ACA | | |
| GAG CCT TGC AAA CCA TGTACT GAG TGC GTT | | |
| GGA CTT CAG TCA ATG AGC GCT CCA TGT GTG | | |
| GAG GCA GAT GAT GCG GTC TGT CGA TGT GCT | | |
| TAC GGA TAC TACCAA GAC GAG ACA ACA GGG | | |
| CGG TGC GAG GCC TGT AGA GTT TGT GAG GCG | | |
| GGC TCC GGG CTG GTG TTT TCA TGT CAA GAC | | |
| AAG CAAAAT ACG GTC TGT GAA GAG TGC CCT | | |
| GAT GGC ACC TAC TCA GAC GAA GCA GAT GCA | | |
| GAA TGC | | |
| GCC TGC CCT ACA GGA CTC TAC ACG CAT AGC | 56 | Truncated LNGFR hinge (TNFR- |
| GGT GAG TGT TGT AAA GCA TGC AAC CTC GGG | | Cys1) |
| GAA GGT GTA GCC CAG CCA TGC GGG GCT AAC | | |
| CAA ACC GTT TGC | | |
| GCTGTGGGCCAGGACACGCAGGAGGTCATCGTGG | 57 | PDGFR-beta extracellular linker |
| TGCCACACTCCTTGCCCTTTAAGGTG | | |

In some embodiments, the chimeric inhibitory receptor further comprises a spacer region between the protein binding domain and the transmembrane domain.

In some embodiments, the spacer region is derived from a protein selected from the group consisting of: CD8α, CD4, CD7, CD28, IgG1, IgG4, FcγRIIIα, LNGFR, and PDGFR. In some embodiments, the spacer region comprises an amino acid sequence selected from the group consisting of:

(SEQ ID NO: 39)
AAAIEVMYPPPYLDNEKSNGTIIHVK
GKHLCPSPLFPGPSKP,
(SEQ ID NO: 40)
ESKYGPPCPSCP,
(SEQ ID NO: 41)
ESKYGPPAPSAP,
(SEQ ID NO: 42)
ESKYGPPCPPCP,
(SEQ ID NO: 43)
EPKSCDKTHTCP,
(SEQ ID NO: 44)
AAAFVPVFLPAKPTTTPAPRPPTPAPT
IASQPLSLRPEACRPAAGGAVHTRGLD
FACDIYIWAPLAGTCGVLLLSLVITLY
CNHRN,
(SEQ ID NO: 45)
TTTPAPRPPTPAPTIALQPLSLRPEAC
RPAAGGAVHTRGLDFACD,
(SEQ ID NO: 46)
ACPTGLYTHSGECCKACNLGEGVAQPC
GANQTVCEPCLDSVTFSDVVSATEPCK
PCTECVGLQSMSAPCVEADDAVCRCAY
GYYQDETTGRCEACRVCEAGSGLVFSC
QDKQNTVCEECPDGTYSDEADAEC,
(SEQ ID NO: 47)
ACPTGLYTHSGECCKACNLGEGVAQP
CGANQTVC,
and
(SEQ ID NO: 48)
AVGQDTQEVIVVPHSLPFKV.

In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 39. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 40. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 41. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 42. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 43. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 44. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 45. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 46. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 47. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 48. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 49.

In some embodiments, the spacer region modulates sensitivity of the chimeric inhibitory receptor. In some embodiments, the spacer region increases sensitivity of the chimeric inhibitory receptor relative to an otherwise identical chimeric inhibitory receptor lacking the spacer region. In some embodiments, the spacer region reduces sensitivity of the chimeric inhibitory receptor relative to an otherwise identical chimeric inhibitory receptor lacking the spacer region. In some embodiments, the spacer region modulates potency of the chimeric inhibitory receptor relative to an otherwise identical chimeric inhibitory receptor lacking the spacer region. In some embodiments, the spacer region increases potency of the chimeric inhibitory receptor relative to an otherwise identical chimeric inhibitory receptor lacking the spacer region. In some embodiments, the spacer region reduces potency of the chimeric inhibitory receptor relative to an otherwise identical chimeric inhibitory receptor lacking the spacer region. In some embodiments, the spacer region modulates basal prevention, attenuation, or inhibition of activation of the tumor-targeting chimeric receptor expressed on the immunomodulatory cell relative to an otherwise identical chimeric inhibitory receptor lacking the spacer region. In some embodiments, the spacer region reduces basal prevention, attenuation, or inhibition relative to an otherwise identical chimeric inhibitory receptor lacking the spacer region. In some embodiments, the spacer region increases basal prevention, attenuation, or inhibition relative to an otherwise identical chimeric inhibitory receptor lacking the spacer region.

In some embodiments, wherein the chimeric inhibitory receptor further comprises an intracellular spacer region positioned between the transmembrane domain and the intracellular signaling domain and operably linked to each of the transmembrane domain and the intracellular signaling domain. In some embodiments, the chimeric inhibitory receptor further comprises an intracellular spacer region positioned between the transmembrane domain and the intracellular signaling domain and physically linked to each of the transmembrane domain and the intracellular signaling domain.

In some embodiments, the intracellular spacer region modulates sensitivity of the chimeric inhibitory receptor relative to an otherwise identical chimeric inhibitory receptor lacking the intracellular spacer region. In some embodiments, the intracellular spacer region increases sensitivity of the chimeric inhibitory receptor relative to an otherwise identical chimeric inhibitory receptor lacking the intracellular spacer region. In some embodiments, the intracellular spacer region reduces sensitivity of the chimeric inhibitory receptor relative to an otherwise identical chimeric inhibitory receptor lacking the intracellular spacer region. In some embodiments, the intracellular spacer region modulates potency of the chimeric inhibitory receptor relative to an otherwise identical chimeric inhibitory receptor lacking the intracellular spacer region.

In some embodiments, the intracellular spacer region increases potency of the chimeric inhibitory receptor relative to an otherwise identical chimeric inhibitory receptor lacking the intracellular spacer region. In some embodiments, the intracellular spacer region reduces potency of the chimeric inhibitory receptor relative to an otherwise identical chimeric inhibitory receptor lacking the intracellular spacer region. In some embodiments, the intracellular spacer region modulates basal prevention, attenuation, or inhibition of activation of the tumor-targeting chimeric receptor expressed on the immunomodulatory cell when expressed on an immunomodulatory cell relative to an otherwise identical chimeric inhibitory receptor lacking the intracellular spacer region. In some embodiments, the intracellular spacer region reduces basal prevention, attenuation, or inhibition relative to an otherwise identical chimeric inhibitory receptor lacking the intracellular spacer region. In some embodiments, the intracellular spacer region increases basal prevention, attenuation, or inhibition relative to an otherwise identical chimeric inhibitory receptor lacking the intracellular spacer region.

Polynucleotides Encoding Inhibitory Chimeric Receptors

In another aspect, presented herein are a polynucleotide or set of polynucleotides encoding an inhibitory chimeric receptor, and a vector comprising such a polynucleotide. When the inhibitory chimeric receptor is a multichain receptor, a set of polynucleotides is used. In this case, the set of polynucleotides can be cloned into a single vector or a plurality of vectors. In some embodiments, the polynucleotide comprises a sequence encoding an inhibitory chimeric receptor, wherein the sequence encoding an extracellular protein binding domain is contiguous with and in the same reading frame as a sequence encoding an intracellular signaling domain and a transmembrane domain.

The polynucleotide can be codon optimized for expression in a mammalian cell. In some embodiments, the entire sequence of the polynucleotide has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleic acid sequences. A variety of codon optimization methods is known in the art, and include, e.g., methods disclosed in at least U.S. Pat. Nos. 5,786,464 and 6,114,148.

The polynucleotide encoding an inhibitory chimeric receptor can be obtained using recombinant methods known in the art, such as, for example by screening libraries from cells expressing the polynucleotide, by deriving it from a vector known to include the same, or by isolating directly from cells and tissues containing the same, using standard techniques. Alternatively, the polynucleotide can be produced synthetically, rather than cloned.

The polynucleotide can be cloned into a vector. In some embodiments, an expression vector known in the art is used. Accordingly, the present disclosure includes retroviral and lentiviral vector constructs expressing an inhibitory chimeric receptor that can be directly transduced into a cell.

The present disclosure also includes an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequence (“UTR”) (e.g., a 3′ and/or 5′ UTR described herein), a 5′ cap (e.g., a 5′ cap described herein) and/or Internal Ribosome Entry Site (IRES) (e.g., an IRES described herein), the nucleic acid to be expressed, and a polyA tail. RNA so produced can efficiently transfect different kinds of cells. In some embodiments, an RNA inhibitory chimeric receptor vector is transduced into a cell, e.g., a T cell or a NK cell, by electroporation.

Cells

In one aspect, the present disclosure provides inhibitory chimeric receptor-modified cells. The cells can be stem cells, progenitor cells, and/or immune cells modified to express an inhibitory chimeric receptor described herein. In some embodiments, a cell line derived from an immune cell is used. Non-limiting examples of cells, as provided herein, include mesenchymal stem cells (MSCs), natural killer (NK) cells, NKT cells, innate lymphoid cells, mast cells, eosinophils, basophils, macrophages, neutrophils, mesenchymal stem cells, dendritic cells, T cells (e.g., CD8+ T cells, CD4+ T cells, gamma-delta T cells, and T regulatory cells (CD4+, FOXP3+, CD25+)) and B cells. In some embodiments, the cell a stem cell, such as pluripotent stem cell, embryonic stem cell, adult stem cell, bone-marrow stem cell, umbilical cord stem cells, or other stem cell.

The cells can be modified to express an inhibitory chimeric receptor provided herein. Accordingly, the present disclosure provides a cell (e.g., a population of cells) engineered to express an inhibitory chimeric receptor, wherein the inhibitory chimeric receptor comprises a protein binding domain, a transmembrane domain, and an inhibitory intracellular signaling domain.

In some embodiments, the immunomodulatory cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell. In some embodiments, the immunomodulatory cell is a CD8+ T cell. In some embodiments, the immunomodulatory cell is a CD4+ T cell. In some embodiments, the immunomodulatory cell is a Natural Killer T (NKT) cell. In some embodiments, the immunomodulatory cell is a Natural Killer (NK) cell.

In some embodiments, the cell is autologous. In some embodiments, the cell is allogeneic.

In some embodiments, an immunomodulatory cell comprises a chimeric inhibitory receptor, wherein the chimeric inhibitory receptor comprises: an extracellular protein binding domain; a transmembrane domain, wherein the transmembrane domain is operably linked to the extracellular protein binding domain; and an intracellular signaling domain, wherein the intracellular signaling domain is operably linked to the transmembrane domain, and wherein upon binding of the protein to the chimeric inhibitory receptor, the chimeric inhibitory receptor prevents, attenuates, or inhibits activation of a tumor-targeting chimeric receptor expressed on the surface of the cell.

In some embodiments, the cell further comprises a tumor-targeting chimeric receptor expressed on the surface of the cell. In some embodiments, the chimeric inhibitory receptor is recombinantly expressed.

In some embodiments, prior to binding of the protein to the chimeric inhibitory receptor, the tumor-targeting chimeric receptor is capable of activating the cell. In some embodiments, upon binding of the protein to the chimeric inhibitory receptor, the chimeric inhibitory receptor suppresses cytokine production from the activated cell. In some embodiments, upon binding of the protein to the chimeric inhibitory receptor, the chimeric inhibitory receptor suppresses a cell-mediated immune response to a target cell, wherein the immune response is induced by activation of the immunomodulatory cell. In some embodiments, the target cell is a tumor cell. In some embodiments, the target cell is a non-tumor cell.

Cells Expressing Multiple Chimeric Receptors

The cells can be modified to express an inhibitory chimeric receptor provided herein. The cells can also be modified to express an inhibitory chimeric receptor (e.g., an iCAR) and a tumor-targeting CAR (e.g., an aCAR). If a cell is modified to express at least one inhibitory chimeric receptor and at least one tumor-targeting CAR, the cells can express multiple inhibitory and/or tumor-targeting chimeric receptor proteins and/or polynucleotides. In some embodiments, the cell expresses at least 1, at least 2, at least 3, at least 4, at least 5, at least 6, at least 7, at least 8, at least 9, or at least 10 or more inhibitory chimeric receptor polynucleotide and/or polypeptide. In some embodiments, the cell contains at least 1, at least 2, at least 3, at least 4, at least 5, at least 6, at least 7, at least 8, at least 9, or at least 10 or more tumor-targeting chimeric receptor polynucleotide and/or polypeptide.

Methods of Preparing Inhibitory Chimeric Receptor-Modified Cells

In one aspect, the present disclosure provides a method of preparing a modified immune cells comprising an inhibitory chimeric receptor for experimental or therapeutic use.

Ex vivo procedures for making therapeutic inhibitory chimeric receptor-modified cells are well known in the art. For example, cells are isolated from a mammal (e.g., a human) and genetically modified (i.e., transduced or transfected in vitro) with a vector expressing a inhibitory chimeric receptor disclosed herein. The inhibitory chimeric receptor-modified cell can be administered to a mammalian recipient to provide a therapeutic benefit. The mammalian recipient may be a human and the inhibitory chimeric receptor-modified cell can be autologous with respect to the recipient. Alternatively, the cells can be allogeneic, syngeneic or xenogeneic with respect to the recipient. The procedure for ex vivo expansion of hematopoietic stem and progenitor cells is described in U.S. Pat. No. 5,199,942, incorporated herein by reference, can be applied to the cells of the present disclosure. Other suitable methods are known in the art; therefore the present disclosure is not limited to any particular method of ex vivo expansion of the cells. Briefly, ex vivo culture and expansion of immune effector cells (e.g., T cells, NK cells) comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In some embodiments, the methods comprise culturing the population of cells (e.g. in cell culture media) to a desired cell density (e.g., a cell density sufficient for a particular cell-based therapy). In some embodiments, the population of cells are cultured in the absence of an agent that represses activity of the repressible protease or in the presence of an agent that represses activity of the repressible protease.

In some embodiments, the population of cells is cultured for a period of time that results in the production of an expanded cell population that comprises at least 2-fold the number of cells of the starting population. In some embodiments, the population of cells is cultured for a period of time that results in the production of an expanded cell population that comprises at least 4-fold the number of cells of the starting population. In some embodiments, the population of cells is cultured for a period of time that results in the production of an expanded cell population that comprises at least 16-fold the number of cells of the starting population.

Methods of Use

Methods for treatment of immune-related disorders, such as cancers, are also encompassed. Said methods include administering an inhibitory chimeric receptor or immunoresponsive inhibitory chimeric receptor-modified cell as described herein. In some embodiments, compositions comprising chimeric receptors or genetically modified immunoresponsive cells that express such chimeric receptors can be provided systemically or directly to a subject for the treatment of a proliferative disorder, such as a cancer.

In one aspect, the present disclosure provides a method of preparing a modified immune cells comprising at least one inhibitory chimeric receptor (e.g., inhibitory chimeric receptor (iCAR)-modified cells) for experimental or therapeutic use. In some embodiments, the modified immune cells further comprise at least one tumor-targeting chimeric receptor (e.g., iCAR and aCAR-modified cells).

In some aspects, methods of use encompass methods of preventing, attenuating, or inhibiting a cell-mediated immune response induced by a chimeric receptor expressed of the surface of an immunomodulatory cell, comprising: engineering the immunomodulatory cell to express the chimeric inhibitory receptor described herein on the surface of the immunomodulatory cell, wherein upon binding of a cognate protein to the chimeric inhibitory receptor, the intracellular signaling domain prevents, attenuates, or inhibits activation of the chimeric receptor. In other aspects, methods of use encompass methods of preventing, attenuating, or inhibiting activation of a chimeric receptor expressed on the surface of an immunomodulatory cell, comprising: contacting an isolated cell or a composition as described herein with a cognate protein of the chimeric inhibitory receptor under conditions suitable for the chimeric inhibitory receptor to bind the cognate protein, wherein upon binding of the protein to the chimeric inhibitory receptor, the intracellular signaling domain prevents, attenuates, or inhibits activation of the chimeric receptor.

In general, the inhibitory chimeric receptor is used to prevent, attenuate, inhibit, or suppress an immune response initiated by a tumor targeting chimeric receptor (e.g., an activating CAR). For example, an immunomodulator cell expresses an inhibitory chimeric antigen that recognizes an antigen target 1 (e.g., a non-tumor antigen) and a tumor-targeting chimeric receptor that recognizes an antigen target 2 (e.g., a tumor target). When the exemplary immunomodulatory cell contacts a target cell, the inhibitory and tumor targeting chimeric receptors may or may not bind to their cognate antigen. In exemplary instances where the target cell is a non-tumor cell that expresses both antigen target 1 and antigen target 2, both the inhibitory chimeric receptor and the tumor-targeting receptor can be activated. In such cases, the activation of the inhibitory chimeric receptor results in the prevention, attenuation, or inhibition of the tumor targeting chimeric receptor signaling and the immunomodulatory cell is not activated. Similarly, in exemplary instances where the target cell is a non-tumor cell that expresses only antigen target 1, only the inhibitory chimeric receptor can be activated. In contrast, in exemplary instances where the target cell is a tumor cell that expresses only antigen target 2, the inhibitory chimeric receptor cannot be activated while the tumor-targeting chimeric receptor can be activated, resulting in signal transduction that results in activation of the immunomodulatory cell.

Attenuation of an immune response initiated by a tumor targeting chimeric receptor can be a decrease or reduction in the activation of the tumor targeting chimeric receptor, a decrease or reduction in the signal transduction of a tumor targeting chimeric receptor, or a decrease or reduction in the activation of the immunomodulatory cell. The inhibitory chimeric receptor can attenuate activation of the tumor targeting chimeric receptor, signal transduction by the tumor targeting chimeric receptor, or activation of the immunomodulatory cell by the tumor targeting chimeric receptor 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold or more as compared to the activation of the tumor targeting chimeric receptor, signal transduction, or activation of the immunomodulatory cell as compared to an immunomodulatory cell lacking an inhibitory chimeric receptor. In some embodiments, attenuation refers to a decrease or reduction of the activity of a tumor targeting chimeric receptor after it has been activated.

Prevention of an immune response initiated by a tumor targeting chimeric receptor can be an inhibition or reduction in the activation of the tumor targeting chimeric receptor, an inhibition or reduction in the signal transduction of a tumor targeting chimeric receptor, or an inhibition or reduction in the activation of the immunomodulatory cell. The inhibitory chimeric receptor can prevent activation of the tumor targeting chimeric receptor, signal transduction by the tumor targeting chimeric receptor, or activation of the immunomodulatory cell by the tumor targeting chimeric receptor by about 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold or more as compared to the activation of the tumor targeting chimeric receptor, signal transduction, or activation of the immunomodulatory cell as compared to an immunomodulatory cell lacking an inhibitory chimeric receptor. In some embodiments, prevention refers to a blockage of the activity of a tumor targeting chimeric receptor before it has been activated.

Inhibition of an immune response initiated by a tumor targeting chimeric receptor can be an inhibition or reduction in the activation of the tumor targeting chimeric receptor, an inhibition or reduction in the signal transduction of a tumor targeting chimeric receptor, or an inhibition or reduction in the activation of the immunomodulatory cell. The inhibitory chimeric receptor can inhibit activation of the tumor targeting chimeric receptor, signal transduction by the tumor targeting chimeric receptor, or activation of the immunomodulatory cell by the tumor targeting chimeric receptor by about 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold or more as compared to the activation of the tumor targeting chimeric receptor, signal transduction, or activation of the immunomodulatory cell as compared to an immunomodulatory cell lacking an inhibitory chimeric receptor. In some embodiments, inhibition refers to a decrease or reduction of the activity of a tumor targeting chimeric receptor before or after it has been activated.

Suppression of an immune response initiated by a tumor targeting chimeric receptor can be an inhibition or reduction in the activation of the tumor targeting chimeric receptor, an inhibition or reduction in the signal transduction of a tumor targeting chimeric receptor, or an inhibition or reduction in the activation of the immunomodulatory cell. The inhibitory chimeric receptor can suppress activation of the tumor targeting chimeric receptor, signal transduction by the tumor targeting chimeric receptor, or activation of the immunomodulatory cell by the tumor targeting chimeric receptor by about 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold or more as compared to the activation of the tumor targeting chimeric receptor, signal transduction, or activation of the immunomodulatory cell as compared to an immunomodulatory cell lacking an inhibitory chimeric receptor. In some embodiments, suppression refers to a decrease or reduction of the activity of a tumor targeting chimeric receptor before or after it has been activated.

The immune response can be cytokine or chemokine production and secretion from an activated immunomodulatory cell. The immune response can be a cell-mediated immune response to a target cell.

In some embodiments, the chimeric inhibitory receptor is capable of suppressing cytokine production from an activated immunomodulatory cell. In some embodiments, the chimeric inhibitory receptor is capable of suppressing a cell-mediated immune response to a target cell, wherein the immune response is induced by activation of the immunomodulatory cell.

In one aspect, the present disclosure provides a type of cell therapy where immune cells are genetically modified to express an inhibitory chimeric receptor provided herein and the modified immune cells are administered to a subject in need thereof.

Thus, in some embodiments, the methods comprise delivering cells of the expanded population of cells to a subject in need of a cell-based therapy to treat a condition or disorder. In some embodiments, the subject is a human subject. In some embodiments, the condition or disorder is an autoimmune condition. In some embodiments, the condition or disorder is an immune related condition. In some embodiments, the condition or disorder is a cancer (e.g., a primary cancer or a metastatic cancer). In some embodiments, the cancer is a solid cancer. In some embodiments, the cancer is a liquid cancer, such as a myeloid disorder.

Pharmaceutical Compositions

The inhibitory chimeric receptor or immunoresponsive cell can be formulated in pharmaceutical compositions. Pharmaceutical compositions of the present disclosure can comprise an inhibitory chimeric receptor (e.g., an iCAR) or immunoresponsive cell (e.g., a plurality of inhibitory chimeric receptor-expressing cells), as described herein, in combination with one or more pharmaceutically or physiologically acceptable carriers, diluents or excipients. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes. In certain embodiments, the composition is directly injected into an organ of interest (e.g., an organ affected by a disorder). Alternatively, the composition may be provided indirectly to the organ of interest, for example, by administration into the circulatory system (e.g., the tumor vasculature). Expansion and differentiation agents can be provided prior to, during, or after administration of the composition to increase production of T cells, NK cells, or CTL cells in vitro or in vivo.

In certain embodiments, the compositions are pharmaceutical compositions comprising genetically modified cells, such as immunoresponsive cells or their progenitors and a pharmaceutically acceptable carrier. Administration can be autologous or heterologous. For example, immunoresponsive cells, or progenitors can be obtained from one subject, and administered to the same subject or a different, compatible subject. In some embodiments, immunoresponsive cells of the present disclosure or their progeny may be derived from peripheral blood cells (e.g., in vivo, ex vivo, or in vitro derived) and may be administered via localized injection, including catheter administration, systemic injection, localized injection, intravenous injection, or parenteral administration. When administering a therapeutic composition of the present disclosure (e.g., a pharmaceutical composition containing a genetically modified cell of the present disclosure), it will generally be formulated in a unit dosage injectable form (solution, suspension, emulsion).

Certain aspects of the present disclosure relate to formulations of compositions comprising chimeric receptors of the present disclosure or genetically modified cells (e.g., immunoresponsive cells of the present disclosure) expressing such chimeric receptors. In some embodiments, compositions of the present disclosure comprising genetically modified cells may be provided as sterile liquid preparations, including without limitation isotonic aqueous solutions, suspensions, emulsions, dispersions, and viscous compositions, which may be buffered to a selected pH. Liquid preparations are typically easier to prepare than gels, other viscous compositions, and solid compositions. Additionally, liquid compositions may be more convenient to administer, especially by injection. In some embodiments, viscous compositions can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (e.g., glycerol, propylene glycol, liquid polyethylene glycol, etc.) and suitable mixtures thereof.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. In some embodiments, compositions of the present disclosure can be isotonic, i.e., having the same osmotic pressure as blood and lacrimal fluid. In some embodiments, the desired isotonicity may be achieved using, for example, sodium chloride, dextrose, boric acid, sodium tartrate, propylene glycol, or other inorganic or organic solutes.

In some embodiments, compositions of the present disclosure may further include various additives that may enhance the stability and sterility of the compositions. Examples of such additives include, without limitation, antimicrobial preservatives, antioxidants, chelating agents, and buffers. In some embodiments, microbial contamination may be prevented by the inclusions of any of various antibacterial and antifungal agents, including without limitation parabens, chlorobutanol, phenol, sorbic acid, and the like. Prolonged absorption of an injectable pharmaceutical formulation of the present disclosure can be brought about by the use of suitable agents that delay absorption, such as aluminum monostearate and gelatin. In some embodiments, sterile injectable solutions can be prepared by incorporating genetically modified cells of the present disclosure in a sufficient amount of the appropriate solvent with various amounts of any other ingredients, as desired. Such compositions may be in admixture with a suitable carrier, diluent, or excipient such as sterile water, physiological saline, glucose, dextrose, or the like. In some embodiments, the compositions can also be lyophilized. The compositions can contain auxiliary substances such as wetting, dispersing agents, pH buffering agents, and antimicrobials depending upon the route of administration and the preparation desired.

In some embodiments, the components of the formulations of the present disclosure are selected to be chemically inert and to not affect the viability or efficacy of the genetically modified cells of the present disclosure.

One consideration concerning the therapeutic use of the genetically modified cells of the present disclosure is the quantity of cells needed to achieve optimal efficacy. In some embodiments, the quantity of cells to be administered will vary for the subject being treated. In certain embodiments, the quantity of genetically modified cells that are administered to a subject in need thereof may range from 1×10⁴ cells to 1×10¹⁰ cells. In some embodiments, the precise quantity of cells that would be considered an effective dose may be based on factors individual to each subject, including their size, age, sex, weight, and condition of the particular subject. Dosages can be readily ascertained by those skilled in the art based on the present disclosure and the knowledge in the art.

Whether it is a polypeptide, antibody, nucleic acid, small molecule or other pharmaceutically useful compound according to the present invention that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

Kits

Certain aspects of the present disclosure relate to kits for the treatment and/or prevention of a cancer or other diseases (e.g., immune-related or autoimmune disorders). In certain embodiments, the kit includes a therapeutic or prophylactic composition comprising an effective amount of one or more chimeric receptors of the present disclosure, isolated nucleic acids of the present disclosure, vectors of the present disclosure, and/or cells of the present disclosure (e.g., immunoresponsive cells). In some embodiments, the kit comprises a sterile container. In some embodiments, such containers can be boxes, ampules, bottles, vials, tubes, bags, pouches, blister-packs, or other suitable container forms known in the art. The container may be made of plastic, glass, laminated paper, metal foil, or other materials suitable for holding medicaments.

In some embodiments, therapeutic or prophylactic composition is provided together with instructions for administering the therapeutic or prophylactic composition to a subject having or at risk of developing a cancer or immune-related disorder. In some embodiments, the instructions may include information about the use of the composition for the treatment and/or prevention of the disorder. In some embodiments, the instructions include, without limitation, a description of the therapeutic or prophylactic composition, a dosage schedule, an administration schedule for treatment or prevention of the disorder or a symptom thereof, precautions, warnings, indications, counter-indications, over-dosage information, adverse reactions, animal pharmacology, clinical studies, and/or references. In some embodiments, the instructions can be printed directly on the container (when present), or as a label applied to the container, or as a separate sheet, pamphlet, card, or folder supplied in or with the container.

### Additional Embodiments

Provided below are enumerated embodiments describing specific embodiments of the invention:
Embodiment 1: A chimeric inhibitory receptor comprising:
   an extracellular protein binding domain;
   a transmembrane domain, wherein the transmembrane domain is operably linked to the extracellular protein binding domain; and
   one or more intracellular signaling domains, wherein the one or more intracellular signaling domains are operably linked to the transmembrane domain, and
      wherein at least one of the one or more intracellular signaling domains is capable of preventing, attenuating, or inhibiting activation of a tumor-targeting chimeric receptor expressed on an immunomodulatory cell.
Embodiment 2: The chimeric inhibitory receptor of embodiment 1, wherein the one or more intracellular signaling domain are each derived from a protein selected from the group consisting of: SLAP1, SLAP2, Dok-1, Dok-2, LAIR1, GRB-2, CD200R, SIRPα, HAVR, GITR, PD-L1, KIR2DL1, KIR2DL2, KIR2DL3, KIR3DL2, CD94, KLRG-1, CEACAM1, LIR2, LIR3, LIR5, SIGLEC-2, and SIGLEC-10.
Embodiment 3: The chimeric inhibitory receptor of any one of embodiments 1 or 2, wherein the transmembrane domain is derived from the same protein as one of the one or more intracellular signaling domains.
Embodiment 4: The chimeric inhibitory receptor of embodiment 3, wherein the transmembrane domain further comprises at least a portion of an extracellular domain of the same protein.
Embodiment 5: The chimeric inhibitory receptor of any one of embodiments 1 or 2, wherein the transmembrane domain is derived from a first protein and the one or more intracellular signaling domains are derived from proteins that are distinct from the first protein.
Embodiment 6: The chimeric inhibitory receptor of any one of embodiments 1-5, wherein one of the one or more intracellular signaling domains is derived from SLAP1.
Embodiment 7: The chimeric inhibitory receptor of embodiment 6, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 4)
PAPAERPLPNPEGLDSDFLAVLSDYPSPDISPPIF
RRGEKLRVISDEGGWWKAISLSTGRESYIPGICVA
RVYHGWLFEGLGRDKAEELLQLPDTKVGSFMIRES
ETKKGFYSLSVRHRQVKHYRIFRLPNNWYYISPRL
TFQCLEDLVNHYSEVADGLCCVLTTPCLTQSTAAP
AVRASSSPVTLRQKTVDWRRVSRLQEDPEGTENPL
GVDESLFSYGLRESIASYLSLTSEDNTSFDRKKKS
ISLMYGGSKRKSSFFSSPPYFED,
or
(SEQ ID NO: 5)
PAPAERPLPNPEGLDSDFLAVLSDYPSPDISPPIF
RRGEKLRVISDEGGWWKAISLSTGRESYIPGICVA
RVYHGWLFEGLGRDKAEELLQLPDTKVGSFMIRES
ETKKGFYSLSVRHRQVKHYRIFRLPNNWYYISPRL
TFQCLEDLVNHYSEVADGLCCVLTTPCLTQSTAAP
AVRASSSPVTLRQKTVDWRRVSRLQEDPEGTENPL
GVDESLFSYGLRESIASYLSLTSEDNTSF.

Embodiment 8: The chimeric inhibitory receptor of embodiment 6, wherein the intracellular signaling domain comprises the amino acid sequence of

(SEQ ID NO: 4)
PAPAERPLPNPEGLDSDFLAVLSDYPSPDISPPIF
RRGEKLRVISDEGGWWKAISLSTGRESYIPGICVA
RVYHGWLFEGLGRDKAEELLQLPDTKVGSFMIRES
ETKKGFYSLSVRHRQVKHYRIFRLPNNWYYISPRL
TFQCLEDLVNHYSEVADGLCCVLTTPCLTQSTAAP
AVRASSSPVTLRQKTVDWRRVSRLQEDPEGTENPL
GVDESLFSYGLRESIASYLSLTSEDNTSFDRKKKS
ISLMYGGSKRKSSFFSSPPYFED,
or
(SEQ ID NO: 5)
PAPAERPLPNPEGLDSDFLAVLSDYPSPDISPPIF
RRGEKLRVISDEGGWWKAISLSTGRESYIPGICVA
RVYHGWLFEGLGRDKAEELLQLPDTKVGSFMIRES
ETKKGFYSLSVRHRQVKHYRIFRLPNNWYYISPRL
TFQCLEDLVNHYSEVADGLCCVLTTPCLTQSTAAP
AVRASSSPVTLRQKTVDWRRVSRLQEDPEGTENPL
GVDESLFSYGLRESIASYLSLTSEDNTSF.

Embodiment 9: The chimeric inhibitory receptor of any one of embodiments 1-5, wherein one of the one or more intracellular signaling domains is derived from SLAP2.
Embodiment 10: The chimeric inhibitory receptor of embodiment 9, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 6)
RKSLPSPSLSSSVQGQGPVTMEAERSKATAVALGS
FPAGGPAELSLRLGEPLTIVSEDGDWWTVLSEVSG
REYNIPSVHVAKVSHGWLYEGLSREKAEELLLLPG
NPGGAFLIRESQTRRGSYSLSVRLSRPASWDRIRH
YRIHCLDNGWLYISPRLTFPSLQALVDHYSELADD
ICCLLKEPCVLQRAGPLPGKDIPLPVTVQRTPLNW
KELDSSLLFSEAATGEESLLSEGLRESLSFYISLN
DEAVSLDDA.

Embodiment 11: The chimeric inhibitory receptor of embodiment 9, wherein the intracellular signaling domain comprises the amino acid sequence of

(SEQ ID NO: 6)
RKSLPSPSLSSSVQGQGPVTMEAERSKATAVALGS
FPAGGPAELSLRLGEPLTIVSEDGDWWTVLSEVSG
REYNIPSVHVAKVSHGWLYEGLSREKAEELLLLPG
NPGGAFLIRESQTRRGSYSLSVRLSRPASWDRIRH
YRIHCLDNGWLYISPRLTFPSLQALVDHYSELADD
ICCLLKEPCVLQRAGPLPGKDIPLPVTVQRTPLNW
KELDSSLLFSEAATGEESLLSEGLRESLSFYISLN
DEAVSLDDA.

Embodiment 12: The chimeric inhibitory receptor of any one of embodiments 1-5, wherein one of the one or more intracellular signaling domains is derived from KIR2DL1.
Embodiment 13: The chimeric inhibitory receptor of embodiment 12, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 60)
HRWCSNKKNAAVMDQESAGNRTANSEDSDEQDPQE
VTYTQLNHCVFTQRKITRPSQRPKTPPTDIIVYTE
LPNAESRSKVVSCP.

Embodiment 14: The chimeric inhibitory receptor of embodiment 12, wherein the intracellular signaling domain comprises the amino acid sequence of

(SEQ ID NO: 60)
HRWCSNKKNAAVMDQESAGNRTANSEDSDEQDPQE
VTYTQLNHCVFTQRKITRPSQRPKTPPTDIIVYTE
LPNAESRSKVVSCP.

Embodiment 15: The chimeric inhibitory receptor of any one of embodiments 1-5, wherein one of the one or more intracellular signaling domains is derived from KLRG-1.
Embodiment 16: The chimeric inhibitory receptor of embodiment 15, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 61)
MTDSVIYSMLELPTATQAQNDYGPQQKSSSSRPSCSCLGSG.

Embodiment 17: The chimeric inhibitory receptor of embodiment 15, wherein the intracellular signaling domain comprises the amino acid sequence of

(SEQ ID NO: 61)
MTDSVIYSMLELPTATQAQNDYGPQQKSSSSRPSCSCLGSG.

Embodiment 18: The chimeric inhibitory receptor of any one of embodiments 1-5, wherein one of the one or more intracellular signaling domains is derived from LAIR1.
Embodiment 19: The chimeric inhibitory receptor of embodiment 18, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 62)
HRQNQIKQGPPRSKDEEQKPQQRPDLAVDVLERTADKATVNGLPEKDRE
TDTSALAAGSSQEVTYAQLDHWALTQRTARAVSPQSTKPMAESITYAAV
ARH.

Embodiment 20: The chimeric inhibitory receptor of embodiment 18, wherein the intracellular signaling domain comprises the amino acid sequence of

(SEQ ID NO: 62)
HRQNQIKQGPPRSKDEEQKPQQRPDLAVDVLERTADKATVNGLPEKDRE
TDTSALAAGSSQEVTYAQLDHWALTQRTARAVSPQSTKPMAESITYAAV
ARH.

Embodiment 21: The chimeric inhibitory receptor of any one of embodiments 1-5, wherein one of the one or more intracellular signaling domains is derived from LIR2.
Embodiment 22: The chimeric inhibitory receptor of embodiment 21, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 63)
LRHRRQGKHWTSTQRKADFQHPAGAVGPEPTDRGLQWRSSPAADAQEEN
LYAAVKDTQPEDGVEMDTRAAASEAPQDVTYAQLHSLTLRRKATEPPPS
QEREPPAEPSIYATLAIH.

Embodiment 23: The chimeric inhibitory receptor of embodiment 21, wherein the intracellular signaling domain comprises the amino acid sequence of

(SEQ ID NO: 63)
LRHRRQGKHWTSTQRKADFQHPAGAVGPEPTDRGLQWRSSPAADAQEEN
LYAAVKDTQPEDGVEMDTRAAASEAPQDVTYAQLHSLTLRRKATEPPPS
QEREPPAEPSIYATLAIH.

Embodiment 24: The chimeric inhibitory receptor of any one of embodiments 1-5, wherein one of the one or more intracellular signaling domains is derived from LIR3.
Embodiment 25: The chimeric inhibitory receptor of embodiment 24, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 64)
RRQRHSKHRTSDQRKTDFQRPAGAAETEPKDRGLLRRSSPAADVQEENL
YAAVKDTQSEDRVELDSQSPHDEDPQAVTYAPVKHSSPRREMASPPSSL
SGEFLDTKDRQVEEDRQMDTEAAASEASQDVTYAQLHSLTLRRKATEPP
PSQEGEPPAEPSIYATLAIH.

Embodiment 26: The chimeric inhibitory receptor of embodiment 24, wherein the intracellular signaling domain comprises the amino acid sequence of

(SEQ ID NO: 64)
RRQRHSKHRTSDQRKTDFQRPAGAAETEPKDRGLLRRSSPAADVQEENL
YAAVKDTQSEDRVELDSQSPHDEDPQAVTYAPVKHSSPRREMASPPSSL
SGEFLDTKDRQVEEDRQMDTEAAASEASQDVTYAQLHSLTLRRKATEPP
PSQEGEPPAEPSIYATLAIH.

Embodiment 27: The chimeric inhibitory receptor of any one of embodiments 1-5, wherein one of the one or more intracellular signaling domains is derived from LIR5.
Embodiment 28: The chimeric inhibitory receptor of embodiment 27, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 65)
QHWRQGKHRTLAQRQADFQRPPGAAEPEPKDGGLQRRSSPAADVQGENF
CAAVKNTQPEDGVEMDTRQSPHDEDPQAVTYAKVKHSRPRREMASPPSP
LSGEFLDTKDRQAEEDRQMDTEAAASEAPQDVTYAQLHSFTLRQKATEP
PPSQEGASPAEPSVYATLAIH.

Embodiment 29: The chimeric inhibitory receptor of embodiment 27, wherein the intracellular signaling domain comprises the amino acid sequence of

(SEQ ID NO: 65)
QHWRQGKHRTLAQRQADFQRPPGAAEPEPKDGGLQRRSSPAADVQGENF
CAAVKNTQPEDGVEMDTRQSPHDEDPQAVTYAKVKHSRPRREMASPPSP
LSGEFLDTKDRQAEEDRQMDTEAAASEAPQDVTYAQLHSFTLRQKATEP
PPSQEGASPAEPSVYATLAIH.

Embodiment 30: The chimeric inhibitory receptor of any one of embodiments 1-5, wherein one of the one or more intracellular signaling domains is derived from SIGLEC-2.
Embodiment 31: The chimeric inhibitory receptor of embodiment 30, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 66)
KLQRRWKRTQSQQGLQENSSGQSFFVRNKKVRRAPLSEGPHSLGCYNPM
MEDGISYTTLRFPEMNIPRTGDAESSEMQRPPPDCDDTVTYSALHKRQV
GDYENVIPDFPEDEGIHYSELIQFGVGERPQAQENVDYVILKH.

Embodiment 32: The chimeric inhibitory receptor of embodiment 30, wherein the intracellular signaling domain comprises the amino acid sequence of

(SEQ ID NO: 66)
KLQRRWKRTQSQQGLQENSSGQSFFVRNKKVRRAPLSEGPHSLGCYNPM
MEDGISYTTLRFPEMNIPRTGDAESSEMQRPPPDCDDTVTYSALHKRQV
GDYENVIPDFPEDEGIHYSELIQFGVGERPQAQENVDYVILKH.

Embodiment 33: The chimeric inhibitory receptor of any one of embodiments 1-5, wherein one of the one or more intracellular signaling domains is derived from SIGLEC-10.
Embodiment 34: The chimeric inhibitory receptor of embodiment 33, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 67)
KILPKRRTQTETPRPRFSRHSTILDYINVVPTAGPLAQKRNQKATPNSP
RTPLPPGAPSPESKKNQKKQYQLPSFPEPKSSTQAPESQESQEELHYAT
LNFPGVRPRPEARMPKGTQADYAEVKFQ.

Embodiment 35: The chimeric inhibitory receptor of embodiment 33, wherein the intracellular signaling domain comprises the amino acid sequence of

(SEQ ID NO: 67)
KILPKRRTQTETPRPRFSRHSTILDYINVVPTAGPLAQKRNQKATPNSP
RTPLPPGAPSPESKKNQKKQYQLPSFPEPKSSTQAPESQESQEELHYAT
LNFPGVRPRPEARMPKGTQADYAEVKFQ.

Embodiment 36: The chimeric inhibitory receptor of any one of embodiments 1-35, wherein the transmembrane domain is derived from a protein selected from the group consisting of: CD8, CD28, CD3, CD4, 4-IBB, OX40, ICOS, 2B4, CD25, CD7, LAX, LAT, LAIR1, GRB-2, Dok-1, Dok-2, SLAP1, SLAP2, CD200R, SIRPα, HAVR, GITR, PD-L1, KIR2DL1, KIR2DL2, KIR2DL3, KIR3DL2, CD94, KLRG-1, CEACAM1, LIR2, LIR3, LIR5, SIGLEC-2, and SIGLEC-10.
Embodiment 37: The chimeric inhibitory receptor of any one of embodiments 1-36, wherein the chimeric inhibitory receptor comprises a transmembrane domain derived from CD28.
Embodiment 38: The chimeric inhibitory receptor of embodiment 37, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 20)
FWVLVVVGGVLACYSLLVTVAFIIFWV.

Embodiment 39: The chimeric inhibitory receptor of embodiment 37, wherein the transmembrane domain comprises the amino acid sequence of

(SEQ ID NO: 20)
FWVLVVVGGVLACYSLLVTVAFIIFWV.

Embodiment 40: The chimeric inhibitory receptor of any one of embodiments 1-36, wherein the chimeric inhibitory receptor comprises a transmembrane domain derived from KIR2DL1.
Embodiment 41: The chimeric inhibitory receptor of embodiment 40, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to ILIGTSVVIILFILLFFLL (SEQ ID NO: 76).
Embodiment 42: The chimeric inhibitory receptor of embodiment 40, wherein the transmembrane domain comprises the amino acid sequence of ILIGTSVVIILFILLFFLL (SEQ ID NO: 76).
Embodiment 43: The chimeric inhibitory receptor of any one of embodiments 1-36, wherein the chimeric inhibitory receptor comprises a transmembrane domain derived from KLRG-1.
Embodiment 44: The chimeric inhibitory receptor of embodiment 43, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VAIALGLLTAVLLSVLLYQWI (SEQ ID NO: 78).
Embodiment 45: The chimeric inhibitory receptor of embodiment 43, wherein the transmembrane domain comprises the amino acid sequence of

(SEQ ID NO: 78)
VAIALGLLTAVLLSVLLYQWI.

Embodiment 46: The chimeric inhibitory receptor of any one of embodiments 1-36, wherein the chimeric inhibitory receptor comprises a transmembrane domain derived from LAIR1.
Embodiment 47: The chimeric inhibitory receptor of embodiment 46, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to ILIGVSVVFLFCLLLLVLFCL (SEQ ID NO: 79).
Embodiment 48: The chimeric inhibitory receptor of embodiment 46, wherein the transmembrane domain comprises the amino acid sequence of

(SEQ ID NO: 79)
ILIGVSVVFLFCLLLLVLFCL.

Embodiment 49: The chimeric inhibitory receptor of any one of embodiments 1-36, wherein the chimeric inhibitory receptor comprises a transmembrane domain derived from LIR2.
Embodiment 50: The chimeric inhibitory receptor of embodiment 49, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VIGILVAVVLLLLLLLLLFLI (SEQ ID NO: 80).
Embodiment 51: The chimeric inhibitory receptor of embodiment 49, wherein the transmembrane domain comprises the amino acid sequence of

(SEQ ID NO: 80)
VIGILVAVVLLLLLLLLLFLI.

Embodiment 52: The chimeric inhibitory receptor of any one of embodiments 1-36, wherein the chimeric inhibitory receptor comprises a transmembrane domain derived from LIR3.
Embodiment 53: The chimeric inhibitory receptor of embodiment 52, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VLIGVSVAFVLLLFLLLFLLL (SEQ ID NO: 81).
Embodiment 54: The chimeric inhibitory receptor of embodiment 52, wherein the transmembrane domain comprises the amino acid sequence of

(SEQ ID NO: 81)
VLIGVSVAFVLLLFLLLFLLL.

Embodiment 55: The chimeric inhibitory receptor of any one of embodiments 1-36, wherein the chimeric inhibitory receptor comprises a transmembrane domain derived from LIR5.
Embodiment 56: The chimeric inhibitory receptor of embodiment 55, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VLIGVLVVSILLLSLLLFLLL (SEQ ID NO: 82).
Embodiment 57: The chimeric inhibitory receptor of embodiment 55, wherein the transmembrane domain comprises the amino acid sequence of

(SEQ ID NO: 82)
VLIGVLVVSILLLSLLLFLLL.

Embodiment 58: The chimeric inhibitory receptor of any one of embodiments 1-36, wherein the chimeric inhibitory receptor comprises a transmembrane domain derived from SIGLEC-2.
Embodiment 59: The chimeric inhibitory receptor of embodiment 58, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VAVGLGSCLAILILAICGL (SEQ ID NO: 83).
Embodiment 60: The chimeric inhibitory receptor of embodiment 58, wherein the transmembrane domain comprises the amino acid sequence of VAVGLGSCLAILILAICGL (SEQ ID NO: 83).
Embodiment 61: The chimeric inhibitory receptor of any one of embodiments 1-36, wherein the chimeric inhibitory receptor comprises a transmembrane domain derived from SIGLEC-10.
Embodiment 62: The chimeric inhibitory receptor of embodiment 61, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to GAFLGIGITALLFLCLALIIM (SEQ ID NO: 84).
Embodiment 63: The chimeric inhibitory receptor of embodiment 61, wherein the transmembrane domain comprises the amino acid sequence of

(SEQ ID NO: 84)
GAFLGIGITALLFLCLALIIM.

Embodiment 64: The chimeric inhibitory receptor of any one of embodiments 1-63, wherein the one or more intracellular signaling domains are two intracellular signaling domains.
Embodiment 65: The chimeric inhibitory receptor of embodiment 64, wherein the chimeric inhibitory receptor comprises a first intracellular signaling domain derived from KIR2DL1 and a second intracellular signaling domain derived from LIR2.
Embodiment 66: The chimeric inhibitory receptor of embodiment 64, wherein the chimeric inhibitory receptor comprises a first intracellular signaling domain derived from KIR2DL1 and a second intracellular signaling domain derived from LIR3.
Embodiment 67: The chimeric inhibitory receptor of embodiment 64, wherein the chimeric inhibitory receptor comprises a first intracellular signaling domain derived from KIR2DL1 and a second intracellular signaling domain derived from LIR5.
Embodiment 68: The chimeric inhibitory receptor of any one of embodiments 65-67, wherein the first intracellular signaling domain further comprises a transmembrane domain derived from KIR2DL1.
Embodiment 69: The chimeric inhibitory receptor of embodiment 64, wherein the chimeric inhibitory receptor comprises a first intracellular signaling domain derived from LIR2 and a second intracellular signaling domain derived from KIR2DL1.
Embodiment 70: The chimeric inhibitory receptor of embodiment 69, wherein the first intracellular signaling domain further comprises a transmembrane domain derived from LIR2.
Embodiment 71: The chimeric inhibitory receptor of embodiment 64, wherein the chimeric inhibitory receptor comprises a first intracellular signaling domain derived from LIR3 and a second intracellular signaling domain derived from KIR2DL1.
Embodiment 72: The chimeric inhibitory receptor of embodiment 71, wherein the first intracellular signaling domain further comprises a transmembrane domain derived from LIR3.
Embodiment 73: The chimeric inhibitory receptor of embodiment 64, wherein the chimeric inhibitory receptor comprises a first intracellular signaling domain derived from LIR5 and a second intracellular signaling domain derived from KIR2DL1.
Embodiment 74: The chimeric inhibitory receptor of embodiment 73, wherein the first intracellular signaling domain further comprises a transmembrane domain derived from LIR5.
Embodiment 75: The chimeric inhibitory receptor of any one of embodiments 1-74, wherein the protein is not expressed on the target tumor.
Embodiment 76: The chimeric inhibitory receptor of any one of embodiments 1-75, wherein the protein is expressed on a non-tumor cell.
Embodiment 77: The chimeric inhibitory receptor of embodiment 76, wherein the protein is expressed on a non-tumor cell derived from a tissue selected from the group consisting of brain, neuronal tissue, endocrine, endothelial, bone, bone marrow, immune system, muscle, lung, liver, gallbladder, pancreas, gastrointestinal tract, kidney, urinary bladder, male reproductive organs, female reproductive organs, adipose, soft tissue, and skin.
Embodiment 78: The chimeric inhibitory receptor of any one of embodiments 1-77, wherein the extracellular protein binding domain comprises a ligand-binding domain.
Embodiment 79: The chimeric inhibitory receptor of any one of embodiments 1-77, wherein the extracellular protein binding domain comprises a receptor-binding domain.
Embodiment 80: The chimeric inhibitory receptor of any one of embodiments 1-77, wherein the extracellular protein binding domain comprises an antigen-binding domain.
Embodiment 81: The chimeric inhibitory receptor of embodiment 80, wherein the antigen-binding domain comprises an antibody, an antigen-binding fragment of an antibody, a F(ab) fragment, a F(ab′) fragment, a single chain variable fragment (scFv), or a single-domain antibody (sdAb).
Embodiment 82: The chimeric inhibitory receptor of embodiment 80, wherein the antigen-binding domain comprises a single chain variable fragment (scFv).
Embodiment 83: The chimeric inhibitory receptor of embodiment 82, wherein each scFv comprises a heavy chain variable domain (VH) and a light chain variable domain (VL).
Embodiment 84: The chimeric inhibitory receptor of embodiment 83, wherein the VH and VL are separated by a peptide linker.
Embodiment 85: The chimeric inhibitory receptor of embodiment 84, wherein the peptide linker comprises an amino acid sequence selected from the group consisting of: GGS (SEQ ID NO: 23), GGSGGS (SEQ ID NO: 24), GGSGGSGGS (SEQ ID NO: 25), GGSGGSGGSGGS (SEQ ID NO: 26), GGSGGSGGSGGSGGS (SEQ ID NO: 27), GGGS (SEQ ID NO: 28), GGGSGGGS (SEQ ID NO: 29), GGGSGGGSGGGS (SEQ ID NO: 30), GGGSGGGSGGGSGGGS (SEQ ID NO: 31), GGGSGGGSGGGSGGGSGGGS (SEQ ID NO: 32), GGGGS (SEQ ID NO: 33), GGGGSGGGGS (SEQ ID NO: 34), GGGGSGGGGSGGGGS (SEQ ID NO: 35), GGGGSGGGGSGGGGSGGGGS (SEQ ID NO: 36), GGGGSGGGGSGGGGSGGGGSGGGGS (SEQ ID NO: 37), and TTTPAPRPPTPAPTIALQPLSLRPEACRPAAGGAVHTRGLDFACDQTTPGERSSLPAFY PGTSGSCSGCGSLSLP (SEQ ID NO: 94).
Embodiment 86: The chimeric inhibitory receptor of any one of embodiments 83-85, wherein the scFv comprises the structure VH-L-VL or VL-L-VH, wherein VH is the heavy chain variable domain, L is the peptide linker, and VL is the light chain variable domain.
Embodiment 87: The chimeric inhibitory receptor of any one of embodiments 1-86, wherein the transmembrane domain is physically linked to the extracellular protein binding domain.
Embodiment 88: The chimeric inhibitory receptor of any one of embodiments 1-87, wherein one of the one or more intracellular signaling domains is physically linked to the transmembrane domain.
Embodiment 89: The chimeric inhibitory receptor of any one of embodiments 1-88, wherein the transmembrane domain is physically linked to the extracellular protein binding domain and one of the one or more intracellular signaling domains is physically linked to the transmembrane domain.
Embodiment 90: The chimeric inhibitory receptor of any one of embodiments 1-89, wherein the extracellular protein binding domain has a high binding affinity.
Embodiment 91: The chimeric inhibitory receptor of any one of embodiments 1-89, wherein extracellular protein binding domain has a low binding affinity.
Embodiment 92: The chimeric inhibitory receptor of any one of embodiments 1-91, wherein the chimeric inhibitory receptor is capable of suppressing cytokine production from an activated immunomodulatory cell.
Embodiment 93: The chimeric inhibitory receptor of any one of embodiments 1-92, wherein the chimeric inhibitory receptor is capable of suppressing a cell-mediated immune response to a target cell, wherein the immune response is induced by activation of the immunomodulatory cell.
Embodiment 94: The chimeric inhibitory receptor of any one of embodiments 1-93, wherein the target cell is a tumor cell.
Embodiment 95: The chimeric inhibitory receptor of any one of embodiments 1-94, wherein the one or more intracellular signaling domains comprise one or more modifications.
Embodiment 96: The chimeric inhibitory receptor of embodiment 95, wherein the one or more modifications modulate sensitivity of the chimeric inhibitory receptor relative to the otherwise identical, unmodified receptor.
Embodiment 97: The chimeric inhibitory receptor of embodiment 95, wherein the one or more modifications increase sensitivity of the chimeric inhibitory receptor relative to the otherwise identical, unmodified receptor.
Embodiment 98: The chimeric inhibitory receptor of embodiment 95, wherein the one or more modifications reduce sensitivity of the chimeric inhibitory receptor relative to the otherwise identical, unmodified receptor.
Embodiment 99: The chimeric inhibitory receptor of any one embodiments 95-98, wherein the one or more modifications modulate potency of the chimeric inhibitory receptor relative to the otherwise identical, unmodified receptor.
Embodiment 100: The chimeric inhibitory receptor of embodiment 99, wherein the one or more modifications increase potency of the chimeric inhibitory receptor relative to the otherwise identical, unmodified receptor.
Embodiment 101: The chimeric inhibitory receptor of embodiment 99, wherein the one or more modifications reduce potency of the chimeric inhibitory receptor relative to the otherwise identical, unmodified receptor.
Embodiment 102: The chimeric inhibitory receptor of any one of embodiments 95-101, wherein the one or more modifications modulate basal prevention, attenuation, or inhibition of activation of the tumor-targeting chimeric receptor when expressed on an immunomodulatory cell relative to the otherwise identical, unmodified receptor.
Embodiment 103: The chimeric inhibitory receptor of embodiment 102, wherein the one or more modifications reduce basal prevention, attenuation, or inhibition relative to the otherwise identical, unmodified receptor.
Embodiment 104: The chimeric inhibitory receptor of embodiment 102, wherein the one or more modifications increase basal prevention, attenuation, or inhibition relative to the otherwise identical, unmodified receptor.
Embodiment 105: The chimeric inhibitory receptor of any one of embodiments 1-104, wherein the chimeric inhibitory receptor further comprises a spacer region positioned between the extracellular protein binding domain and the transmembrane domain and operably linked to each of the extracellular protein binding domain and the transmembrane domain.
Embodiment 106: The chimeric inhibitory receptor of any one of embodiments 1-104, wherein the chimeric inhibitory receptor further comprises a spacer region positioned between the extracellular protein binding domain and the transmembrane domain and physically linked to each of the extracellular protein binding and the transmembrane domain.
Embodiment 107: The chimeric inhibitory receptor of embodiment 105, wherein the spacer region is derived from a protein selected from the group consisting of: CD8α, CD4, CD7, CD28, IgG1, IgG4, FcγRIIIα, LNGFR, and PDGFR.
Embodiment 108: The chimeric inhibitory receptor of embodiment 105, wherein the spacer region comprises an amino acid sequence selected from the group consisting of:

(SEQ ID NO: 39)
AAAIEVMYPPPYLDNEKSNGTIIHVKGKHLCPSPLFPGPSKP,
(SEQ ID NO: 40)
ESKYGPPCPSCP,
(SEQ ID NO: 41)
ESKYGPPAPSAP,
(SEQ ID NO: 42)
ESKYGPPCPPCP,
(SEQ ID NO: 43)
EPKSCDKTHTCP,
(SEQ ID NO: 44)
AAAFVPVFLPAKPTTTPAPRPPTPAPTIASQPLSLRPEACRPAAGGAV
HTRGLDFACDIYIWAPLAGTCGVLLLSLVITLYCNHRN,
(SEQ ID NO: 46)
ACPTGLYTHSGECCKACNLGEGVAQPCGANQTVCEPCLDSVTFSDVVS
ATEPCKPCTECVGLQSMSAPCVEADDAVCRCAYGYYQDETTGRCEACR
VCEAGSGLVFSCQDKQNTVCEECPDGTYSDEADAEC,
(SEQ ID NO: 47)
ACPTGLYTHSGECCKACNLGEGVAQPCGANQTVC,
and
(SEQ ID NO: 48)
AVGQDTQEVIVVPHSLPFKV.

Embodiment 109: The chimeric inhibitory receptor of any one of embodiments 105-108, wherein the spacer region modulates sensitivity of the chimeric inhibitory receptor relative to an otherwise identical chimeric inhibitory receptor lacking the spacer region.
Embodiment 110: The chimeric inhibitory receptor of embodiment 109, wherein the spacer region increases sensitivity of the chimeric inhibitory receptor relative to an otherwise identical chimeric inhibitory receptor lacking the spacer region.
Embodiment 111: The chimeric inhibitory receptor of embodiment 109, wherein the spacer region reduces sensitivity of the chimeric inhibitory receptor relative to an otherwise identical chimeric inhibitory receptor lacking the spacer region.
Embodiment 112: The chimeric inhibitory receptor of any one of embodiments 105-111, wherein the spacer region modulates potency of the chimeric inhibitory receptor relative to an otherwise identical chimeric inhibitory receptor lacking the spacer region.
Embodiment 113: The chimeric inhibitory receptor of embodiment 112, wherein the spacer region increases potency of the chimeric inhibitory receptor relative to an otherwise identical chimeric inhibitory receptor lacking the spacer region.
Embodiment 114: The chimeric inhibitory receptor of embodiment 112, wherein the spacer region reduces potency of the chimeric inhibitory receptor relative to an otherwise identical chimeric inhibitory receptor lacking the spacer region.
Embodiment 115: The chimeric inhibitory receptor of any one of embodiments 105-114, wherein the spacer region modulates basal prevention, attenuation, or inhibition of activation of the tumor-targeting chimeric receptor when expressed on an immunomodulatory cell relative to an otherwise identical chimeric inhibitory receptor lacking the spacer region.
Embodiment 116: The chimeric inhibitory receptor of embodiment 115, wherein the spacer region reduces basal prevention, attenuation, or inhibition relative to an otherwise identical chimeric inhibitory receptor lacking the spacer region.
Embodiment 117: The chimeric inhibitory receptor of embodiment 115, wherein the spacer region increases basal prevention, attenuation, or inhibition relative to an otherwise identical chimeric inhibitory receptor lacking the spacer region.
Embodiment 118: The chimeric inhibitory receptor of any one of embodiments 1-117, wherein the chimeric inhibitory receptor further comprises an intracellular spacer region positioned between the transmembrane domain and one of the one or more intracellular signaling domains and operably linked to each of the transmembrane domain and one of the one or more intracellular signaling domains.
Embodiment 119: The chimeric inhibitory receptor of any one of embodiments 1-117, wherein the chimeric inhibitory receptor further comprises an intracellular spacer region positioned between the transmembrane domain and one of the one or more intracellular signaling domains and physically linked to each of the transmembrane domain and one of the one or more intracellular signaling domains.
Embodiment 120: The chimeric inhibitory receptor of embodiment 118, wherein the intracellular spacer region modulates sensitivity of the chimeric inhibitory receptor relative to an otherwise identical chimeric inhibitory receptor lacking the intracellular spacer region.
Embodiment 121: The chimeric inhibitory receptor of embodiment 120, wherein the intracellular spacer region increases sensitivity of the chimeric inhibitory receptor relative to an otherwise identical chimeric inhibitory receptor lacking the intracellular spacer region.
Embodiment 122: The chimeric inhibitory receptor of embodiment 120, wherein the intracellular spacer region reduces sensitivity of the chimeric inhibitory receptor relative to an otherwise identical chimeric inhibitory receptor lacking the intracellular spacer region.
Embodiment 123: The chimeric inhibitory receptor of any one of embodiments 118-122, wherein the intracellular spacer region modulates potency of the chimeric inhibitory receptor relative to an otherwise identical chimeric inhibitory receptor lacking the intracellular spacer region.
Embodiment 124: The chimeric inhibitory receptor of embodiment 123, wherein the intracellular spacer region increases potency of the chimeric inhibitory receptor relative to an otherwise identical chimeric inhibitory receptor lacking the intracellular spacer region.
Embodiment 125: The chimeric inhibitory receptor of embodiment 123, wherein the intracellular spacer region reduces potency of the chimeric inhibitory receptor relative to an otherwise identical chimeric inhibitory receptor lacking the intracellular spacer region.
Embodiment 126: The chimeric inhibitory receptor of any one of embodiments 118-125, herein the intracellular spacer region modulates basal prevention, attenuation, or inhibition of activation of the tumor-targeting chimeric receptor when expressed on an immunomodulatory cell relative to an otherwise identical chimeric inhibitory receptor lacking the intracellular spacer region.
Embodiment 127: The chimeric inhibitory receptor of embodiment 126, wherein the intracellular spacer region reduces basal prevention, attenuation, or inhibition relative to an otherwise identical chimeric inhibitory receptor lacking the intracellular spacer region.
Embodiment 128: The chimeric inhibitory receptor of embodiment 126, wherein the intracellular spacer region increases basal prevention, attenuation, or inhibition relative to an otherwise identical chimeric inhibitory receptor lacking the intracellular spacer region.
Embodiment 129: The chimeric inhibitory receptor of any one of embodiments 1-128, wherein the inhibitory chimeric receptor further comprises an enzymatic inhibitory domain.
Embodiment 130: The chimeric inhibitory receptor of embodiment 129, wherein the enzymatic inhibitory domain is capable of preventing, attenuating, or inhibiting activation of a tumor-targeting chimeric receptor when expressed on an immunomodulatory cell relative to an otherwise identical chimeric inhibitory receptor lacking the enzymatic inhibitory domain.
Embodiment 131: The chimeric inhibitory receptor of embodiment 129 or embodiment 130, wherein the enzymatic inhibitory domain comprises an enzyme catalytic domain.
Embodiment 132: The chimeric inhibitory receptor of embodiment 131, wherein the enzyme catalytic domain is derived from an enzyme selected from the group consisting of: CSK, SHP-1, PTEN, CD45, CD148, PTP-MEG1, PTP-PEST, c-CBL, CBL-b, PTPN22, LAR, PTPH1, SHIP-1, and RasGAP.
Embodiment 133: The chimeric inhibitory receptor of any one of embodiments 129-132, wherein the enzymatic inhibitory domain comprises one or more modifications that modulate basal prevention, attenuation, or inhibition.
Embodiment 134: The chimeric inhibitory receptor of embodiment 133, wherein the one or more modifications reduce basal prevention, attenuation, or inhibition relative to an otherwise identical enzymatic inhibitory domain lacking the one or more modifications.
Embodiment 135: The chimeric inhibitory receptor of embodiment 133, wherein the one or more modifications increase basal prevention, attenuation, or inhibition relative to an otherwise identical enzymatic inhibitory domain lacking the one or more modifications.
Embodiment 136: The chimeric inhibitory receptor of any one of embodiments 1-135, wherein the tumor-targeting chimeric receptor is a chimeric antigen receptor (CAR) or an engineered T cell receptor (TCR).
Embodiment 137: The chimeric inhibitory receptor of any one of embodiments 1-136, wherein the immunomodulatory cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell.
Embodiment 138: The chimeric inhibitory receptor of any one of embodiments 1-136, wherein the immunomodulatory cell is a Natural Killer (NK) cell.
Embodiment 139: A composition comprising the chimeric inhibitory receptor of any one of embodiments 1-138 and a pharmaceutically acceptable carrier.
Embodiment 140: An engineered nucleic acid encoding the chimeric inhibitory receptor of any one of embodiments 1-138.
Embodiment 141: An expression vector comprising the engineered nucleic acid of embodiment 140.
Embodiment 142: A composition comprising the engineered nucleic acid of embodiment 140 or the expression vector of embodiment 141, and a pharmaceutically acceptable carrier
Embodiment 143: An isolated immunomodulatory cell comprising the chimeric inhibitory receptor of any one of embodiments 1-138.
Embodiment 144: The isolated cell of embodiment 143, wherein the cell further comprises a tumor-targeting chimeric receptor expressed on the surface of the cell.
Embodiment 145: The isolated cell of embodiment 144, wherein upon binding of the protein to the chimeric inhibitory receptor, the chimeric inhibitory receptor prevents, attenuates, or inhibits activation of the tumor-targeting chimeric receptor relative to an otherwise identical cell lacking a chimeric inhibitory receptor.
Embodiment 146: An isolated immunomodulatory cell comprising a chimeric inhibitory receptor, wherein the chimeric inhibitory receptor comprises:
   an extracellular protein binding domain,
   a transmembrane domain, wherein the transmembrane domain is operably linked to the extracellular protein binding domain, and
   one or more intracellular signaling domains, wherein the one or more intracellular signaling domains are operably linked to the transmembrane domain, and wherein the one or more intracellular signaling domain are each derived from a protein selected from the group consisting of: SLAP1, SLAP2, Dok-1, Dok-2, LAIR1, GRB-2, CD200R, SIRPα, HAVR, GITR, PD-L1, KIR2DL1, KIR2DL2, KIR2DL3, KIR3DL2, CD94, KLRG-1, CEACAM1, LIR2, LIR3, LIR5, SIGLEC-2, and SIGLEC-10; and
   wherein upon binding of the protein to the chimeric inhibitory receptor, the chimeric inhibitory receptor prevents, attenuates, or inhibits activation of a tumor-targeting chimeric receptor expressed on the surface of the cell.
Embodiment 147: The isolated cell of embodiment 146, wherein the cell further comprises a tumor-targeting chimeric receptor expressed on the surface of the cell.
Embodiment 148: An isolated cell comprising:
   (a) a chimeric inhibitory receptor, wherein and the chimeric inhibitory receptor comprises:
      an extracellular protein binding domain,
      a transmembrane domain, wherein the transmembrane domain is operably linked to the extracellular protein binding domain, and
      one or more intracellular signaling domains, wherein the one or more intracellular signaling domains are operably linked to the transmembrane domain, and wherein the one or more intracellular signaling domain are each derived from a protein selected from the group consisting of: SLAP1, SLAP2, Dok-1, Dok-2, LAIR1, GRB-2, CD200R, SIRPα, HAVR, GITR, PD-L1, KIR2DL1, KIR2DL2, KIR2DL3, KIR3DL2, CD94, KLRG-1, CEACAM1, LIR2, LIR3, LIR5, SIGLEC-2, and SIGLEC-10; and
   (b) a tumor-targeting chimeric receptor expressed on the surface of the cell,
   wherein upon binding of the protein to the chimeric inhibitory receptor, the chimeric inhibitory receptor prevents, attenuates, or inhibits activation of the tumor-targeting chimeric receptor.
Embodiment 149: The isolated cell of any one of embodiments 143-148, wherein the chimeric inhibitory receptor is recombinantly expressed.
Embodiment 150: The isolated cell of any one of embodiments 143-149, wherein the chimeric inhibitory receptor is expressed from a vector or a selected locus from the genome of the cell.
Embodiment 151: The isolated cell of any one of embodiments 143-150, wherein the tumor-targeting chimeric receptor is a chimeric antigen receptor (CAR) or an engineered T cell receptor.
Embodiment 152: The cell of any one of embodiments 143-151, wherein prior to binding of the protein to the chimeric inhibitory receptor, the tumor-targeting chimeric receptor is capable of activating the cell.
Embodiment 153: The cell of any one of embodiments 143-152, wherein upon binding of the protein to the chimeric inhibitory receptor, the chimeric inhibitory receptor suppresses cytokine production from the activated cell.
Embodiment 154: The cell of any one of embodiments 143-153, wherein upon binding of the protein to the chimeric inhibitory receptor, the chimeric inhibitory receptor suppresses a cell-mediated immune response to a target cell, wherein the immune response is induced by activation of the immunomodulatory cell.
Embodiment 155: The cell of any one of embodiments 143-154, wherein the transmembrane domain is physically linked to the extracellular protein binding domain.
Embodiment 156: The cell of any one of embodiments 143-154, wherein the intracellular signaling domain is physically linked to the transmembrane domain.
Embodiment 157: The cell of any one of embodiments 143-154, wherein the transmembrane domain is physically linked to the extracellular protein binding domain and one of the one or more intracellular signaling domains is physically linked to the transmembrane domain.
Embodiment 158: The isolated cell of any one of embodiments 143-154, wherein the target cell is a tumor cell.
Embodiment 159: The isolated cell of any one of embodiments 143-158, wherein the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell.
Embodiment 160: The isolated cell of any one of embodiments 143-158, wherein the cell is a Natural Killer (NK) cell.
Embodiment 161: The isolated cell of any one of embodiments 143-160, wherein the cell is autologous.
Embodiment 162: The isolated cell of any one of embodiments 143-160, wherein the cell is allogeneic.
Embodiment 163: A composition comprising the isolated cell of any one of embodiments 143-162 and a pharmaceutically acceptable carrier.
Embodiment 164: A method of preventing, attenuating, or inhibiting a cell-mediated immune response induced by a tumor-targeting chimeric receptor expressed of the surface of an immunomodulatory cell, comprising:

engineering the immunomodulatory cell to express the chimeric inhibitory receptor of any one of embodiments 1-138 on the surface of the immunomodulatory cell, wherein upon binding of a cognate antigen to the chimeric inhibitory receptor, the intracellular signaling domain prevents, attenuates, or inhibits activation of the tumor-targeting chimeric receptor.
Embodiment 165: A method of preventing, attenuating, or inhibiting activation of a tumor-targeting chimeric receptor expressed on the surface of an immunomodulatory cell, comprising:
   contacting the isolated cell of any one of embodiments 143-162 or the composition of embodiment 163 with a cognate antigen of the chimeric inhibitory receptor under conditions suitable for the chimeric inhibitory receptor to bind the cognate antigen,
   wherein upon binding of the antigen to the chimeric inhibitory receptor, the intracellular signaling domain prevents, attenuates, or inhibits activation of the tumor-targeting chimeric receptor.
Embodiment 166: The method of embodiment 164 or embodiment 165, wherein the tumor-targeting chimeric receptor is a chimeric antigen receptor (CAR) or an engineered T cell receptor.
Embodiment 167: The method of embodiment 166, wherein the CAR binds one or more antigens expressed on the surface of a tumor cell.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

### Example 1: Inhibitory Chimeric Receptor with a SLAP Signaling Domain Reduces T Cell Activation

### Methods and Materials

### T Cell Transduction and Expansion

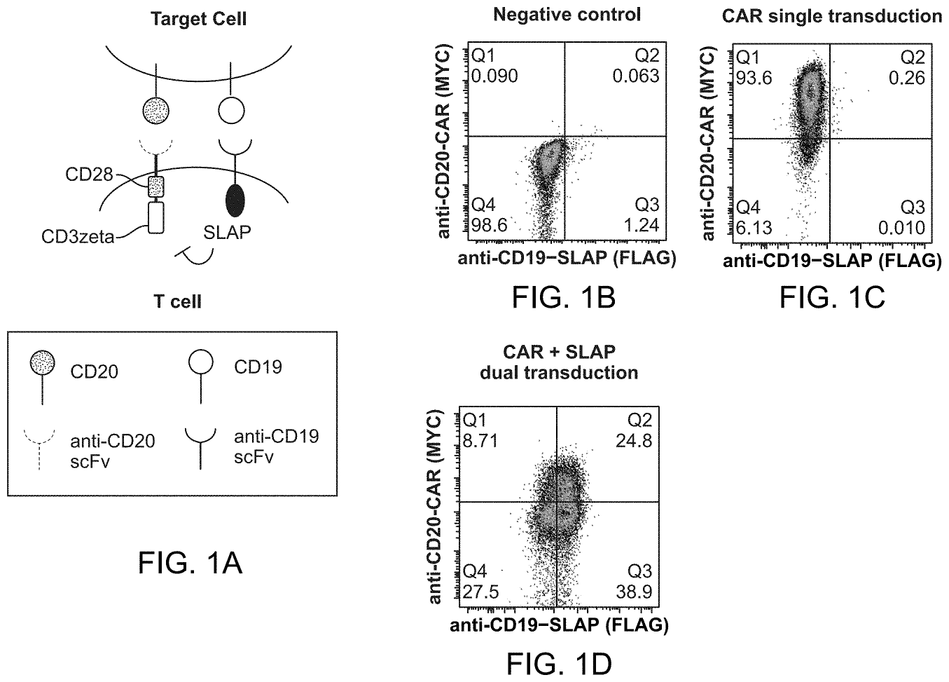

An inhibitory chimeric receptor (iCAR) with a SLAP1 (Src-like adaptor protein-1) intracellular signaling domain was synthesized. The inhibitory chimeric receptor comprised an IgGK secretion signal, an anti-CD19 scFv with a FLAG tag, a CD8 hinge domain, a CD28 transmembrane domain, and a SLAP1 intracellular signaling domain. The FLAG tag was fused to the N-terminus of the scFv (after the signal sequence) in the iCAR. A tumor-targeting CAR (an activating CAR, aCAR) was also constructed with a CD8 secretion signal, an anti-CD20 scFv with a Myc tag, a CD8 hinge domain, a CD28 transmembrane domain, and CD28 and CD3ζ intracellular signaling domains. The Myc tag was fused to the C-terminus of the scFv in the hinge region in the aCAR. An exemplary diagram of a T cell co-expressing an anti-CD19-SLAP iCAR and an anti-CD20-CD28/CD3ζ aCAR contacting a target cell expressing CD19 and CD20 is shown in FIG. 1A.

Table 9 provides the full sequences of the inhibitory chimeric receptor and tumor-targeting chimeric receptor synthesized.

TABLE 9
inhibitory chimeric receptor and tumor-targeting chimeric receptor sequences

| SEQ | | |
|---|---|---|
| ID NO | Name | Sequence |
| 58 | Anti-CD19- | METDTLLLWVLLLWVPGSTGAGGSDYKDDDDKGGSEVKLQESGPGLVAPSQSLSVTCTVSGVSLPD |
| | CD8 hinge- | YGVSWIRQPPRKGLEWLGVIWGSETTYYNSALKSRLTIIKDNSKSQVFLKMNSLQTDDTAIYYCAK |
| | CD28 TM- | HYYYGGSYAMDYWGQGTSVTVSSGGGGSGGGGSGGGGSDIQMTQTTSSLSASLGDRVTISCRASQD |
| | SLAP1 iCAR | ISKYLNWYQQKPDGTVKLLIYHTSRLHSGVPSRFSGSGSGTDYSLTISNLEQEDIATYFCQQGNTL |
| | | PYTFGGGTKLEITTTTPAPRPPTPAPTIALQPLSLRPEACRPAAGGAVHTRGLDFACDFWVLVVVG |
| | | GVLACYSLLVTVAFIIFWVPAPAERPLPNPEGLDSDFLAVLSDYPSPDISPPIFRRGEKLRVISDE |
| | | GGWWKAISLSTGRESYIPGICVARVYHGWLFEGLGRDKAEELLQLPDTKVGSFMIRESETKKGFYS |
| | | LSVRHRQVKHYRIFRLPNNWYYISPRLTFQCLEDLVNHYSEVADGLCCVLTTPCLTQSTAAPAVRA |
| | | SSSPVTLRQKTVDWRRVSRLQEDPEGTENPLGVDESLFSYGLRESIASYLSLTSEDNTSF |
| 59 | anti-CD20- | MALPVTALLLPLALLLHAARPQVQLVQSGAEVKKPGASVKVSCKASGYTFTNYWMHWVRQAPGQGL |
| | CD28/CD3 ξ | EWMGFITPTTGYPEYNQKFKDRVTMTADKSTSTAYMELSSLRSEDTAVYYCARRKVGKGVYYALDY |
| | aCAR | WGQGTTVTVSSGGGGSGGGGSGGGGSDIQMTQSPSSLSASVGDRVTITCRASGNIHNYLAWYQQKP |
| | | GKVPKLLIYNTKTLADGVPSRFSGSGSGTDYTLTISSLQPEDVATYYCQHFWSSPWTFGGGTKVEI |
| | | KEQKLISEEDLNGAATTTPAPRPPTPAPTIALQPLSLRPEACRPAAGGAVHTRGLDFACDFWVLVV |
| | | VGGVLACYSLLVTVAFIIFWVRSKRSRLLHSDYMNMTPRRPGPTRKHYQPYAPPRDFAAYRSRVKF |
| | | SRSADAPAYKQGQNQLYNELNLGRREEYDVLDKRRGRDPEMGGKPRRKNPQEGLYNELQKDKMAEA |
| | | YSEIGMKGERRRGKGHDGLYQGLSTATKDTYDALHMQALPPR |

### T Cell Transduction

On day 1, 1×10⁶ purified CD4+/CD8+ T-cells were thawed and stimulated with 3×10⁶ Dynabeads, then cultured in 1 mL Optimizer CTS T-cell expansion media (Gibco) with 0.2 ug/mL IL-2. T cells were singly or co-transduced on day 2 with lentivirus (100K each, as quantified by GoStix (Tekara)) encoding constitutive expression of the anti-CD20 activating CAR (aCAR) or the anti-CD19 inhibitory CAR (iCAR).

On day 3, the Dynabeads were removed by magnet. The T-cells were counted and passaged (0.5×10⁶ cells/mL). An aliquot of these cells was stained with PE conjugated anti-MYC and BV421 conjugated anti-FLAG antibodies (corresponding to the aCAR and the iCAR, respectively), and their transgene expression quantified using an LX CytoFlex Flow Cytometry machine. During subsequent expansion, cells were passaged every two days (0.5×10⁶ cells/mL).

### T Cell Co-Culture Assay

On day 8, the T-cells were counted and distributed into a 96-well plate for co-culture assays. Each well contained 5×10⁵ Raji target cells stained with cell trace violet dye (Invitrogen) and 5×10⁵ aCAR expressing T cells. Co-cultures were incubated (37° C., 5% CO₂) for 18 hrs.

On day 9, the co-culture supernatant was collected and cytokines in the media were measured using a Human magnetic Luminex assay (R&D systems) and MAGPIX analyzer (Millipore Sigma).

### Results

The ability of an iCAR to reduce or inhibit T cell activation in a T cell expressing an iCAR and an aCAR that each bind different antigens was assessed. An exemplary diagram of a T cell co-expressing an anti-CD20-SLAP iCAR and an anti-CD19 aCAR contacting a target cell expressing CD19 and CD20 is shown in FIG. 1A. The cells transduced with the anti-CD19−SLAP iCAR and anti-CD20 aCAR showed high levels of surface expression in primary T cells. T cells transduced with only the aCAR showed high aCAR expression and no iCAR expression (FIG. 1C), while T cells co-transduced with both the aCAR and iCAR showed high levels of expression of both CAR proteins (FIG. 1D). The negative control cells showed no expression of either construct (FIG. 1B).

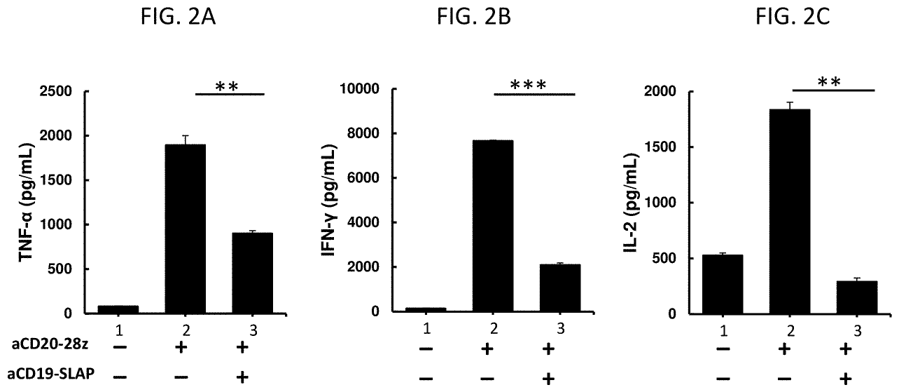

The anti-CD19−SLAP iCAR suppressed the T cell cytokine production induced by the anti-CD20 aCAR (aCD20-28z) after co-culture with Raji cells expressing CD19 and CD20. Co-culture of the Raji cells with anti-CD20 aCAR T cells induced TNF-α, IFN-γ, and IL-2 production (FIGS. 2A, 2B, and 2C, respectively). However, T cells expressing both the anti-CD20 aCAR and the anti-CD19 SLAP iCAR had significantly reduced TNF-α, IFN-γ, and IL-2 production after co-culture with the Raji target cells (**p>0.01, *** p>0.001). Thus, binding of the iCAR to its cognate ligand on the target cell successfully reduced the aCAR-induced cytokine production.

Thus, an anti-CD19-SLAP fusion (iCAR) was expressed at high levels in lentivirus transduced CD4+ and CD8+ T-cells without subsequent enrichment. Importantly, high levels of co-expression of iCAR and aCAR were observed after co-transduction. In addition, the CD19−SLAP iCAR suppressed T-cell activation responses (production of the cytokines TNF-α, IFN-γ, and IL-2) when the iCAR and aCAR target different cell surface ligands (CD19 and CD20, respectively).

### Example 2: Inhibitory Chimeric Receptors with KIR2DL1, KLRG1, LAIR, LIR2, LIR3, LIR5, SIGLEC-2, or SIGLEC-10 Signaling Domains Reduce T Cell Activation

### Methods and Materials

### T Cell Transduction and Expansion

Inhibitory chimeric receptors (iCARs) with KIR2DL1, KLRG1, LAIR, LIR2, LIR3, LIR5, SIGLEC-2, or SIGLEC-10 intracellular signaling domains are synthesized. The inhibitory chimeric receptors each comprise a CD8 signal, a pelB signal (excluding SIGLEC-2 and SIGLEC-10, which only comprise a CD8 signal), an anti-HER2 scFv with a V5 tag, a CD8 hinge domain, and a transmembrane domain and intracellular signaling domain pairing as illustrated in Table 10. The V5 tag is fused to the C-terminus of the scFv in the iCAR. A tumor-targeting CAR (an activating CAR, aCAR) is also constructed with a CD8 secretion signal, an anti-CD20 scFv with a Myc tag, a CD8 hinge domain, a CD28 transmembrane domain, and CD28 and CD3ζ intracellular signaling domains. The Myc tag is fused to the C-terminus of the scFv in the hinge region in the aCAR.

Table 10 provides the transmembrane domain and intracellular signaling domain pairings of this study.

TABLE 10
Transmembrane domain and
intracellular signaling domain pairings

| Transmembrane | Intracellular |
|---|---|
| Domain | signaling domain |
| KIR2DL1 | KIR2DL1 |
| LIR1 | KLRG1 |
| KLRG1 | KLRG1 |
| LAIR1 | LAIR1 |
| LIR2 | LIR2 |
| LIR3 | LIR3 |
| LIR5 | LIR5 |
| SIGLEC-2 | SIGLEC-2 |
| SIGLEC-10 | SIGLEC-10 |

Table 11 provides the full sequences of the inhibitory chimeric receptors and tumor-targeting chimeric receptor.

TABLE 11
inhibitory chimeric receptors and tumor-targeting chimeric receptor sequences

| SEQ ID NO | Name | Sequence |
|---|---|---|
| 95 | Anti-HER2- | MALPVTALLLPLALLLHAARPKYLLPTAAAGLLLLAAQPAMAQVQLVQSGA |
| | CD8 hinge- | EVKKPGESLKISCKGSGYSFTSYWIAWVRQMPGKGLEYMGLIYPGDSDTKY |
| | KIR2DL1 TM- | SPSFQGQVTISVDKSVSTAYLQWSSLKPSDSAVYFCARHDVGYCTDRTCAK |
| | KIR2DL1 | WPEYFQHWGQGTLTVSSGGGGSGGGGSGGGGSQSVLTQPPSVSAAPGQK |
| | iCAR | VTISCSGSSSNIGNNYVSWYQQLPGTAPKLLIYDHTNRPAGVPDRFSGSKSGT |
| | | SASLAISGFRSEDEADYYCASWDYTLSGWVFGGGTKLTVLGGKPIPNPLLGL |
| | | DSTNGAATTTPAPRPPTPAPTIALQPLSLRPEACRPAAGGAVHTRGLDFACDI |
| | | LIGTSVVIILFILLFFLLHRWCSNKKNAAVMDQESAGNRTANSEDSDEQDPQE |
| | | VTYTQLNHCVFTQRKITRPSQRPKTPPTDIIVYTELPNAESRSKVVSCP |
| 96 | Anti-HER2- | MALPVTALLLPLALLLHAARPKYLLPTAAAGLLLLAAQPAMAQVQLVQSGA |
| | CD8 hinge- | EVKKPGESLKISCKGSGYSFTSYWIAWVRQMPGKGLEYMGLIYPGDSDTKY |
| | LIR1 TM- | SPSFQGQVTISVDKSVSTAYLQWSSLKPSDSAVYFCARHDVGYCTDRTCAK |
| | KLRG1 iCAR | WPEYFQHWGQGTLVTVSSGGGGSGGGGSGGGGSQSVLTQPPSVSAAPGQK |
| | | VTISCSGSSSNIGNNYVSWYQQLPGTAPKLLIYDHTNRPAGVPDRFSGSKSGT |
| | | SASLAISGFRSEDEADYYCASWDYTLSGWVFGGGTKLTVLGGKPIPNPLLGL |
| | | DSTNGAATTTPAPRPPTPAPTIALQPLSLRPEACRPAAGGAVHTRGLDFACDV |
| | | IGILVAVILLLLLLLLLFLIMTDSVIYSMLELPTATQAQNDYGPQQKSSSSRPSC |
| | | SCLGSG |
| 97 | Anti-HER2- | MALPVTALLLPLALLLHAARPKYLLPTAAAGLLLLAAQPAMAQVQLVQSGA |
| | CD8 hinge- | EVKKPGESLKISCKGSGYSFTSYWIAWVRQMPGKGLEYMGLIYPGDSDTKY |
| | KLRG1 TM- | SPSFQGQVTISVDKSVSTAYLQWSSLKPSDSAVYFCARHDVGYCTDRTCAK |
| | KLRG1 iCAR | WPEYFQHWGQGTLVTVSSGGGGSGGGGSGGGGSQSVLTQPPSVSAAPGQK |
| | | VTISCSGSSSNIGNNYVSWYQQLPGTAPKLLIYDHTNRPAGVPDRFSGSKSGT |
| | | SASLAISGFRSEDEADYYCASWDYTLSGWVFGGGTKLTVLGGKPIPNPLLGL |
| | | DSTNGAATTTPAPRPPTPAPTIALQPLSLRPEACRPAAGGAVHTRGLDFACDV |
| | | AIALGLLTAVLLSVLLYQWIMTDSVIYSMLELPTATQAQNDYGPQQKSSSSR |
| | | PSCSCLGSG |
| 98 | Anti-HER2- | MALPVTALLLPLALLLHAARPKYLLPTAAAGLLLLAAQPAMAQVQLVQSGA |
| | CD8 hinge- | EVKKPGESLKISCKGSGYSFTSYWIAWVRQMPGKGLEYMGLIYPGDSDTKY |
| | LAIR1 TM- | SPSFQGQVTISVDKSVSTAYLQWSSLKPSDSAVYFCARHDVGYCTDRTCAK |
| | LAIR1 iCAR | WPEYFQHWGQGTLVTVSSGGGGSGGGGSGGGGSQSVLTQPPSVSAAPGQK |
| | | VTISCSGSSSNIGNNYVSWYQQLPGTAPKLLIYDHTNRPAGVPDRFSGSKSGT |
| | | SASLAISGFRSEDEADYYCASWDYTLSGWVFGGGTKLTVLGGKPIPNPLLGL |
| | | DSTNGAATTTPAPRPPTPAPTIALQPLSLRPEACRPAAGGAVHTRGLDFACDI |
| | | LIGVSVVFLFCLLLLVLFCLHRQNQIKQGPPRSKDEEQKPQQRPDLAVDVLER |
| | | TADKATVNGLPEKDRETDTSALAAGSSQEVTYAQLDHWALTQRTARAVSP |
| | | QSTKPMAESITYAAVARH |
| 99 | Anti-HER2- | MALPVTALLLPLALLLHAARPKYLLPTAAAGLLLLAAQPAMAQVQLVQSGA |
| | CD8 hinge- | EVKKPGESLKISCKGSGYSFTSYWIAWVRQMPGKGLEYMGLIYPGDSDTKY |
| | LIR2 TM- | SPSFQGQVTISVDKSVSTAYLQWSSLKPSDSAVYFCARHDVGYCTDRTCAK |
| | LIR2 iCAR | WPEYFQHWGQGTLVTVSSGGGGSGGGGSGGGGSQSVLTQPPSVSAAPGQK |
| | | VTISCSGSSSNIGNNYVSWYQQLPGTAPKLLIYDHTNRPAGVPDRFSGSKSGT |
| | | SASLAISGFRSEDEADYYCASWDYTLSGWVFGGGTKLTVLGGKPIPNPLLGL |
| | | DSTNGAATTTPAPRPPTPAPTIALQPLSLRPEACRPAAGGAVHTRGLDFACDV |
| | | IGILVAVVLLLLLLLLLFLILRHRRQGKHWTSTQRKADFQHPAGAVGPEPTDR |
| | | GLQWRSSPAADAQEENLYAAVKDTQPEDGVEMDTRAAASEAPQDVTYAQL |
| | | HSLTLRRKATEPPPSQEREPPAEPSIYATLAIH |
| 100 | Anti-HER2- | MALPVTALLLPLALLLHAARPKYLLPTAAAGLLLLAAQPAMAQVQLVQSGA |
| | CD8 hinge- | EVKKPGESLKISCKGSGYSFTSYWIAWVRQMPGKGLEYMGLIYPGDSDTKY |
| | LIR3 TM- | SPSFQGQVTISVDKSVSTAYLQWSSLKPSDSAVYFCARHDVGYCTDRTCAK |
| | LIR3 iCAR | WPEYFQHWGQGTLVTVSSGGGGSGGGGSGGGGSQSVLTQPPSVSAAPGQK |
| | | VTISCSGSSSNIGNNYVSWYQQLPGTAPKLLIYDHTNRPAGVPDRFSGSKSGT |
| | | SASLAISGFRSEDEADYYCASWDYTLSGWVFGGGTKLTVLGGKPIPNPLLGL |
| | | DSTNGAATTTPAPRPPTPAPTIALQPLSLRPEACRPAAGGAVHTRGLDFACDV |
| | | LIGVSVAFVLLLFLLLFLLLRRQRHSKHRTSDQRKTDFQRPAGAAETEPKDR |
| | | GLLRRSSPAADVQEENLYAAVKDTQSEDRVELDSQSPHDEDPQAVTYAPVK |
| | | HSSPRREMASPPSSLSGEFLDTKDRQVEEDRQMDTEAAASEASQDVTYAQL |
| | | HSLTLRRKATEPPPSQEGEPPAEPSIYATLAIH |
| 101 | Anti-HER2- | MALPVTALLLPLALLLHAARPKYLLPTAAAGLLLLAAQPAMAQVQLVQSGA |
| | CD8 hinge- | EVKKPGESLKISCKGSGYSFTSYWIAWVRQMPGKGLEYMGLIYPGDSDTKY |
| | LIR5 TM- | SPSFQGQVTISVDKSVSTAYLQWSSLKPSDSAVYFCARHDVGYCTDRTCAK |
| | LIR5 iCAR | WPEYFQHWGQGTLVTVSSGGGGSGGGGSGGGGSQSVLTQPPSVSAAPGQK |
| | | VTISCSGSSSNIGNNYVSWYQQLPGTAPKLLIYDHTNRPAGVPDRFSGSKSGT |
| | | SASLAISGFRSEDEADYYCASWDYTLSGWVFGGGTKLTVLGGKPIPNPLLGL |
| | | DSTNGAATTTPAPRPPTPAPTIALQPLSLRPEACRPAAGGAVHTRGLDFACDV |
| | | LIGVLVVSILLLSLLLFLLLQHWRQGKHRTLAQRQADFQRPPGAAEPEPKDG |
| | | GLQRRSSPAADVQGENFCAAVKNTQPEDGVEMDTRQSPHDEDPQAVTYAK |
| | | VKHSRPRREMASPPSPLSGEFLDTKDRQAEEDRQMDTEAAASEAPQDVTYA |
| | | QLHSFTLRQKATEPPPSQEGASPAEPSVYATLAIH |
| 102 | Anti-HER2- | MALPVTALLLPLALLLHAARPKYLLPTAAAGLLLLAAQPAMAQVQLVQSGA |
| | CD8 hinge- | EVKKPGESLKISCKGSGYSFTSYWIAWVRQMPGKGLEYMGLIYPGDSDTKY |
| | SIGLEC-2 | SPSFQGQVTISVDKSVSTAYLQWSSLKPSDSAVYFCARHDVGYCTDRTCAK |
| | TM-SIGLEC- | WPEYFQHWGQGTLVTVSSGGGGSGGGGSGGGGSQSVLTQPPSVSAAPGQK |
| | 2 iCAR | VTISCSGSSSNIGNNYVSWYQQLPGTAPKLLIYDHTNRPAGVPDRFSGSKSGT |
| | | SASLAISGFRSEDEADYYCASWDYTLSGWVFGGGTKLTVLGGKPIPNPLLGL |
| | | DSTNGAATTTPAPRPPTPAPTIALQPLSLRPEACRPAAGGAVHTRGLDFACDV |
| | | AVGLGSCLAILILAICGLKLQRRWKRTQSQQGLQENSSGQSFFVRNKKVRRA |
| | | PLSEGPHSLGCYNPMMEDGISYTTLRFPEMNIPRTGDAESSEMQRPPPDCDDT |
| | | VTYSALHKRQVGDYENVIPDFPEDEGIHYSELIQFGVGERPQAQENVDYVIL |
| | | KH |
| 103 | Anti-HER2- | MALPVTALLLPLALLLHAARPKYLLPTAAAGLLLLAAQPAMAQVQLVQSGA |
| | CD8 hinge- | EVKKPGESLKISCKGSGYSFTSYWIAWVRQMPGKGLEYMGLIYPGDSDTKY |
| | SIGLEC-10 | SPSFQGQVTISVDKSVSTAYLQWSSLKPSDSAVYFCARHDVGYCTDRTCAK |
| | TM-SIGLEC- | WPEYFQHWGQGTLVTVSSGGGGSGGGGSGGGGSQSVLTQPPSVSAAPGQK |
| | 10 iCAR | VTISCSGSSSNIGNNYVSWYQQLPGTAPKLLIYDHTNRPAGVPDRFSGSKSGT |
| | | SASLAISGFRSEDEADYYCASWDYTLSGWVFGGGTKLTVLGGKPIPNPLLGL |
| | | DSTNGAATTTPAPRPPTPAPTIALQPLSLRPEACRPAAGGAVHTRGLDFACDG |
| | | AFLGIGITALLFLCLALIIMKILPKRRTQTETPRPRFSRHSTILDYINVVPTAGPL |
| | | AQKRNQKATPNSPRTPLPPGAPSPESKKNQKKQYQLPSFPEPKSSTQAPESQE |
| | | SQEELHYATLNFPGVRPRPEARMPKGTQADYAEVKFQ |
| 59 | anti-CD20- | MALPVTALLLPLALLLHAARPQVQLVQSGAEVKKPGASVKVSCKASGYTFT |
| | CD28/CD3 ξ | NYWMHWVRQAPGQGLEWMGFITPTTGYPEYNQKFKDRVTMTADKSTSTA |
| | aCAR | YMELSSLRSEDTAVYYCARRKVGKGVYYALDYWGQGTTVTVSSGGGGSGG |
| | | GGSGGGGSDIQMTQSPSSLSASVGDRVTITCRASGNIHNYLAWYQQKPGKVP |
| | | KLLIYNTKTLADGVPSRFSGSGSGTDYTLTISSLQPEDVATYYCQHFWSSPWT |
| | | FGGGTKVEIKEQKLISEEDLNGAATTTPAPRPPTPAPTIALQPSLRPEACRPA |
| | | AGGAVHTRGLDFACDFWVLVVVGGVLACYSLLVTVAFIIFWVRSKRSRLLH |
| | | SDYMNMTPRRPGPTRKHYQPYAPPRDFAAYRSRVKFSRSADAPAYKQGQN |
| | | QLYNELNLGRREEYDVLDKRRGRDPEMGGKPRRKNPQEGLYNELQKDKMA |
| | | EAYSEIGMKGERRRGKGHDGLYQGLSTATKDTYDALHMQALPPR |

### T Cell Transduction

On day 1, 1×10⁶ purified CD4+/CD8+ T-cells are thawed and stimulated with 3×10⁶ Dynabeads, then cultured in 1 mL Optimizer CTS T-cell expansion media (Gibco) with 0.2 ug/mL IL-2. T cells are singly or co-transduced on day 2 with lentivirus (100K each, as quantified by GoStix (Tekara)) encoding constitutive expression of the anti-CD20 activating CAR (aCAR) or the anti-HER2 inhibitory CAR (iCAR).

On day 3, the Dynabeads are removed by magnet. The T-cells are counted and passaged (0.5×10⁶ cells/mL). An aliquot of these cells is stained with PE conjugated anti-MYC and BV421 conjugated anti-V5 antibodies (corresponding to the aCAR and the iCAR, respectively), and their transgene expression quantified using an LX CytoFlex Flow Cytometry machine. During subsequent expansion, cells are passaged every two days (0.5×10⁶ cells/mL).

### T Cell Co-Culture Assay

On day 8, the T-cells are counted and distributed into a 96-well plate for co-culture assays. Two populations of Raji cells are tested: a parental line, which endogenously expresses CD20+, and an exogenous HER overexpressing Raji line (CD20+Her2+). Each well contained 5×10⁴ Raji target cells stained with cell trace violet dye (Invitrogen) and 5×10⁴ aCAR expressing T cells. Co-cultures are incubated (37° C., 5% CO₂) for 18 hrs.

On day 9, the co-culture supernatant is collected and cytokines in the media are measured using a Human magnetic Luminex assay (R&D systems) and MAGPIX analyzer (Millipore Sigma).

### Results

The ability of an iCAR to reduce or inhibit T cell activation in a T cell expressing an iCAR and an aCAR that each bind different antigens is assessed.

The anti-HER2 iCARs having KIR2DL1, KLRG1, LAIR, LIR2, LIR3, LIR5, SIGLEC-2, or SIGLEC-10 derived inhibitory intracellular signaling domains suppress the T cell cytokine production induced by the anti-CD20 aCAR (aCD20-28z) after co-culture with Raji cells expressing HER2 and CD20. Co-culture of the Raji cells with anti-CD20 aCAR T cells induced TNF-α, IFN-γ, and IL-2 production. However, T cells expressing both the anti-CD20 aCAR and the anti-HER2 iCARs having KIR2DL1, KLRG1, LAIR, LIR2, LIR3, LIR5, SIGLEC-2, or SIGLEC-10 derived inhibitory intracellular signaling domains have significantly reduced TNF-α, IFN-γ, and IL-2 production after co-culture with the Raji target cells. Thus, binding of the iCAR to its cognate ligand on the target cell successfully reduces the aCAR-induced cytokine production.

Anti-HER2 iCARs having KIR2DL1, KLRG1, LAIR, LIR2, LIR3, LIR5, SIGLEC-2, or SIGLEC-10 derived inhibitory intracellular signaling domains are expressed at high levels in lentivirus transduced CD4+ and CD8+ T-cells without subsequent enrichment. High levels of co-expression of iCAR and aCAR are observed after co-transduction. In addition, the anti-HER2 iCARs having KIR2DL1, KLRG1, LAIR, LIR2, LIR3, LIR5, SIGLEC-2, or SIGLEC-10 derived inhibitory intracellular signaling domains suppress T-cell activation responses (production of the cytokines TNF-α, IFN-γ, and IL-2) when the iCAR and aCAR target different cell surface ligands (HER2 and CD20, respectively).

### Example 3: Inhibitory Chimeric Receptors with KIR2DL1, KLRG1, LAIR, LIR2, LIR3, LIR5, SIGLEC-2, or SIGLEC-10 Signaling Domains Reduce NK Cell Activation

### Methods and Materials

### NK Cell Transduction and Expansion

Inhibitory chimeric receptors (iCARs) with KIR2DL1, KLRG1, LAIR, LIR2, LIR3, LIR5, SIGLEC-2, or SIGLEC-10 intracellular signaling domains are synthesized as described in Example 2 above.

NK cells are expanded for 10 days with mitomycin C-treated K562 feeder cells, followed by transduction with 7.5×10⁵ pg of each lentivirus for aCAR and iCAR constructs. Sequences for the constructs to be assessed are shown in Table 11 above. After 4 days, puromycin is added to cells for selection.

### NK Cell Cytotoxicity Assay

After an additional 3 days, cytotoxicity assays are performed by co-incubating engineered NK cells and target cells: parental Raji cells (WT) or Raji cells engineered to overexpress Her2 antigens. Engineered NK cells are incubated either with (1) each target cell type separately at a ratio of 25,000 NK cells to 50,000 Raji cells in triplicate; or (2) as a mixture of 25,000 Raji Her2 only and 25,000 dual antigen Her2+ Raji cells co-incubated with 25,000 NK cells of the indicated type in a 1:1:1 ratio (dual antigen targets were stained with different membrane dyes, allowing them to be distinguished by flow). After overnight incubation, cells are stained with viability dyes and counted via flow cytometry. The target cell reduction is quantified as 100%×(1−No. Targets/No. Targets (NV)).

### Results

The ability of an iCAR to reduce or inhibit NK cell activation in an NK cell expressing an iCAR and an aCAR that each bind different antigens is assessed.

The anti-HER2 iCARs having KIR2DL1, KLRG1, LAIR, LIR2, LIR3, LIR5, SIGLEC-2, or SIGLEC-10 derived inhibitory intracellular signaling domains suppress the NK cell-mediated cytotoxicity of the anti-CD20 aCAR (aCD20-28z) after co-culture with Raji cells expressing HER2 and CD20. Co-culture of the Raji target cells with anti-CD20 aCAR NK cells induced cytotoxicity of parental target cells. However, NK cells expressing both the anti-CD20 aCAR and the anti-HER2 iCARs having KIR2DL1, KLRG1, LAIR, LIR2, LIR3, LIR5, SIGLEC-2, or SIGLEC-10 derived inhibitory intracellular signaling domains have reduced cytotoxicity after co-culture with the Raji target cells. Thus, binding of the iCAR to its cognate ligand on the target cell successfully reduces aCAR-induced cytotoxicity.

Anti-HER2 iCARs having KIR2DL1, KLRG1, LAIR, LIR2, LIR3, LIR5, SIGLEC-2, or SIGLEC-10 derived inhibitory intracellular signaling domains are expressed at high levels in lentivirus transduced NK cell without subsequent enrichment. High levels of co-expression of iCAR and aCAR are observed after co-transduction. In addition, the anti-HER2 iCARs having KIR2DL1, KLRG1, LAIR, LIR2, LIR3, LIR5, SIGLEC-2, or SIGLEC-10 derived inhibitory intracellular signaling domains suppress NK cell activity (NK cell-mediated cytotoxicity) when the iCAR and aCAR target different cell surface ligands (HER2 and CD20, respectively).

### Example 4: Assessment of Various Inhibitory Chimeric Receptors in Reducing NK Cell Activation

### Methods and Materials

Individual iCAR and aCAR constructs were packaged into lentiviral particles and used to transduce primary NK cells after 10 d expansion with K562 feeder cells with 500 U/mL IL-2 and 20 ng/uL IL-15. Virus amounts were set by p24 titer (750,000 pg per transduction). iCAR constructs contained puroR cassettes, so puromycin was added to NK cell cultures from day 4 to 7 post transduction, at which time expression was assessed by flow cytometry and NK cells were transferred to a microwell plate for killing assays with 12,500 NK cells and 50,000 total tumor cells. NK cells were cultured with (1) tumor cells expressing aCAR antigen FLT3 only, (2) tumor cells expressing both aCAR antigen FLT3 and iCAR antigen EMCN, or (3) both tumor cell types mixed. After 16-18 hrs, cultures were analyzed by flow cytometry and remaining live targets cells of each type were counted. aCAR-mediated killing (basal subtracted) of a given NK cell type was quantified by first calculating total killing (reduction of targets compared to a target-only condition), and then subtracting total killing by control (iCAR-only) NK cells. iCAR-mediated protection was quantified as the change in aCAR-mediated killing between targets with or without iCAR antigen. Killing assay supernatant was analyzed for TNFa secretion, and aCAR and iCAR performance metrics were calculated analogously to killing. For expression analysis, iCARs were stained with aV5-Alexafluor 647 and aCARs with aFLAG-BV-421. Cells were assigned to 4 quadrants based on iCAR+/− and aCAR+/− expression states, allowing us to assess “% aCAR+iCAR+” and “% not aCAR+iCAR−” (aCAR+iCAR− are ungated and potentially toxic CAR-NK cells and are to be avoided). To further analyze expression level, we measured median fluorescence intensity (MFI) of aCAR and iCAR of the aCAR+iCAR+ subpopulation, which we normalized by the MFI of untransduced NK cells in the respective fluorescence channels. For each iCAR, 1-3 biological replicates were performed (shown as different points with the same marker type). X and Y error lines (where applicable): +/− standard error of the mean.

The anti-EMCN iCAR constructs assessed used the formats shown in Table 12 with reference to the intracellular domain. The anti-FLT3 aCAR construct assessed is also shown in Table 12.

TABLE 12
Inhibitory chimeric receptors and tumor-targeting chimeric receptor sequences

| SEQ ID NO | Name | Sequence |
|---|---|---|
| 104 | Anti-EMCN- | MALPVTALLLPLALLLHAARPQVQLKESGPGLVQPSQTLSLTCTVSGFSLSRY |
| | CD8 hinge- | DMHWVRQPPGQGLEWMGVIWGNGNTHYHSALKSRLSISRDTSKSQVFLKMNSL |
| | KIR2DL1 TM- | QTEDTAIYFCTLRIKDWGPGTMVTVSSGGGGSGGGGSGGGGSDIVMTQTPPSLS |
| | KIR2DL1 | VALGQSVSISCKSSQSLVASDENTYLNWLLQSPGRSPKRLIYQVSKLDSGVPDRFSG |
| | iCAR (CD8 SS | SGSEKDFTLKISRVEAEDLGVYYCLQGIHLPWTFGGGTKLELKQ [symbol] |
| | bold; EMCN | [symbol] TTTPAPRPPTPAPTIALQPLSLRPEACRPAAGGAVHTRGLDFACDILI |
| | scFv italics; | GTSVVIILFILLFFLLHRWCSNKKNAAVMDQESAGNRTANSEDSDEQDPQEV |
| | V5 + NGAA | TYTQLNHCVFTQRKITRPSQRPKTPPTDIIVYTELPNAESRSKVVSCP |
| | linker bold | |
| | italics) | |
| 105 | Anti-EMCN- | MALPVTALLLPLALLLHAARPQVQLKESGPGLVQPSQTLSLTCTVSGFSLSRY |
| | CD8 hinge- | DMHWVRQPPGQGLEWMGVIWGNGNTHYHSALKSRLSISRDTSKSQVFLKM |
| | LIR1 TM- | NSLQTEDTAIYFCTLRIKDWGPGTMVTVSSGGGGSGGGGSGGGGSDIVMTQ |
| | KLRG1 iCAR | TPPSLSVALGQSVSISCKSSQSLVASDENTYLNWLLQSPGRSPKRLIYQVSKL |
| | | DSGVPDRFSGSGSEKDFTLKISRVEAEDLGVYYCLQGIHLPWTFGGGTKLEL |
| | | KGKPIPNPLLGLDSTNGAATTTPAPRPPTPAPTIALQPLSLRPEACRPAAGGAV |
| | | HTRGLDFACDVIGILVAVILLLLLLLLLFLIMTDSVIYSMLELPTATQAQNDYG |
| | | PQQKSSSSRPSCSCLGSG |
| 106 | Anti-EMCN- | MALPVTALLLPLALLLHAARPQVQLKESGPGLVQPSQTLSLTCTVSGFSLSRY |
| | CD8 hinge- | DMHWVRQPPGQGLEWMGVIWGNGNTHYHSALKSRLSISRDTSKSQVFLKM |
| | KLRG1 TM- | NSLQTEDTAIYFCTLRIKDWGPGTMVTVSSGGGGSGGGGSGGGGSDIVMTQ |
| | KLRG1 iCAR | TPPSLSVALGQSVSISCKSSQSLVASDENTYLNWLLQSPGRSPKRLIYQVSKL |
| | | DSGVPDRFSGSGSEKDFTLKISRVEAEDLGVYYCLQGIHLPWTFGGGTKLEL |
| | | KGKPIPNPLLGLDSTNGAATTTPAPRPPTPAPTIALQPLSLRPEACRPAAGGAV |
| | | HTRGLDFACDVAIALGLLTAVLLSVLLYQWIMTDSVIYSMLELPTATQAQND |
| | | YGPQQKSSSSRPSCSCLGSG |
| 107 | Anti-EMCN- | MALPVTALLLPLALLLHAARPQVQLKESGPGLVQPSQTLSLTCTVSGFSLSRY |
| | CD8 hinge- | DMHWVRQPPGQGLEWMGVIWGNGNTHYHSALKSRLSISRDTSKSQVFLKM |
| | LAIR1 TM- | NSLQTEDTAIYFCTLRIKDWGPGTMVTVSSGGGGSGGGGSGGGGSDIVMTQ |
| | LAIR1 iCAR | TPPSLSVALGQSVSISCKSSQSLVASDENTYLNWLLQSPGRSPKRLIYQVSKL |
| | | DSGVPDRFSGSGSEKDFTLKISRVEAEDLGVYYCLQGIHLPWTFGGGTKLEL |
| | | KGKPIPNPLLGLDSTNGAATTTPAPRPPTPAPTIALQPLSLRPEACRPAAGGAV |
| | | HTRGLDFACDILIGVSVVFLFCLLLLVLFCLHRQNQIKQGPPRSKDEEQKPQQ |
| | | RPDLAVDVLERTADKATVNGLPEKDRETDTSALAAGSSQEVTYAQLDHWA |
| | | LTQRTARAVSPQSTKPMAESITYAAVARH |
| 108 | Anti-EMCN- | MALPVTALLLPLALLLHAARPQVQLKESGPGLVQPSQTLSLTCTVSGFSLSRY |
| | CD8 hinge- | DMHWVRQPPGQGLEWMGVIWGNGNTHYHSALKSRLSISRDTSKSQVFLKM |
| | LIR2 TM- | NSLQTEDTAIYFCTLRIKDWGPGTMVTVSSGGGGSGGGGSGGGGSDIVMTQ |
| | LIR2 iCAR | TPPSLSVALGQSVSISCKSSQSLVASDENTYLNWLLQSPGRSPKRLIYQVSKL |
| | | DSGVPDRFSGSGSEKDFTLKISRVEAEDLGVYYCLQGIHLPWTFGGGTKLEL |
| | | KGKPIPNPLLGLDSTNGAATTTPAPRPPTPAPTIALQPLSLRPEACRPAAGGAV |
| | | HTRGLDFACDVIGILVAVVLLLLLLLLLFLILRHRRQGKHWTSTQRKADFQH |
| | | PAGAVGPEPTDRGLQWRSSPAADAQEENLYAAVKDTQPEDGVEMDTRAAA |
| | | SEAPQDVTYAQLHSLTLRRKATEPPPSQEREPPAEPSIYATLAIH |
| 109 | Anti-EMCN- | MALPVTALLLPLALLLHAARPQVQLKESGPGLVQPSQTLSLTCTVSGFSLSRY |
| | CD8 hinge- | DMHWVRQPPGQGLEWMGVIWGNGNTHYHSALKSRLSISRDTSKSQVFLKM |
| | LIR3 TM- | NSLQTEDTAIYFCTLRIKDWGPGTMVTVSSGGGGSGGGGSGGGGSDIVMTQ |
| | LIR3 iCAR | TPPSLSVALGQSVSISCKSSQSLVASDENTYLNWLLQSPGRSPKRLIYQVSKL |
| | | DSGVPDRFSGSGSEKDFTLKISRVEAEDLGVYYCLQGIHLPWTFGGGTKLEL |
| | | KGKPIPNPLLGLDSTNGAATTTPAPRPPTPAPTIALQPLSLRPEACRPAAGGAV |
| | | HTRGLDFACDVLIGVSVAFVLLLFLLLFLLLRRQRHSKHRTSDQRKTDFQRP |
| | | AGAAETEPKDRGLLRRSSPAADVQEENLYAAVKDTQSEDRVELDSQSPHDE |
| | | DPQAVTYAPVKHSSPRREMASPPSSLSGEFLDTKDRQVEEDRQMDTEAAASE |
| | | ASQDVTYAQLHSLTLRRKATEPPPSQEGEPPAEPSIYATLAIH |
| 110 | Anti-EMCN- | MALPVTALLLPLALLLHAARPQVQLKESGPGLVQPSQTLSLTCTVSGFSLSRY |
| | CD8 hinge- | DMHWVRQPPGQGLEWMGVIWGNGNTHYHSALKSRLSISRDTSKSQVFLKM |
| | LIR5 TM- | NSLQTEDTAIYFCTLRIKDWGPGTMVTVSSGGGGSGGGGSGGGGSDIVMTQ |
| | LIR5 iCAR | TPPSLSVALGQSVSISCKSSQSLVASDENTYLNWLLQSPGRSPKRLIYQVSKL |
| | | DSGVPDRFSGSGSEKDFTLKISRVEAEDLGVYYCLQGIHLPWTFGGGTKLEL |
| | | KGKPIPNPLLGLDSTNGAATTTPAPRPPTPAPTIALQPLSLRPEACRPAAGGAV |
| | | HTRGLDFACDVLIGVLVVSILLLSLLLFLLLQHWRQGKHRTLAQRQADFQRP |
| | | PGAAEPEPKDGGLQRRSSPAADVQGENFCAAVKNTQPEDGVEMDTRQSPHD |
| | | EDPQAVTYAKVKHSRPRREMASPPSPLSGEFLDTKDRQAEEDRQMDTEAAA |
| | | SEAPQDVTYAQLHSFTLRQKATEPPPSQEGASPAEPSVYATLAIH |
| 111 | Anti-EMCN- | MALPVTALLLPLALLLHAARPQVQLKESGPGLVQPSQTLSLTCTVSGFSLSRY |
| | CD8 hinge- | DMHWVRQPPGQGLEWMGVIWGNGNTHYHSALKSRLSISRDTSKSQVFLKM |
| | SIGLEC-2 | NSLQTEDTAIYFCTLRIKDWGPGTMVTVSSGGGGSGGGGSGGGGSDIVMTQ |
| | TM-SIGLEC- | TPPSLSVALGQSVSISCKSSQSLVASDENTYLNWLLQSPGRSPKRLIYQVSKL |
| | 2 iCAR | DSGVPDRFSGSGSEKDFTLKISRVEAEDLGVYYCLQGIHLPWTFGGGTKLEL |
| | | KGKPIPNPLLGLDSTNGAATTTPAPRPPTPAPTIALQPLSLRPEACRPAAGGAV |
| | | HTRGLDFACDVAVGLGSCLAILILAICGLKLQRRWKRTQSQQGLQENSSGQS |
| | | FFVRNKKVRRAPLSEGPHSLGCYNPMMEDGISYTTLRFPEMNIPRTGDAESSE |
| | | MQRPPPDCDDTVTYSALHKRQVGDYENVIPDFPEDEGIHYSELIQFGVGERP |
| | | QAQENVDYVILKH |
| 112 | Anti-EMCN- | MALPVTALLLPLALLLHAARPQVQLKESGPGLVQPSQTLSLTCTVSGFSLSRY |
| | CD8 hinge- | DMHWVRQPPGQGLEWMGVIWGNGNTHYHSALKSRLSISRDTSKSQVFLKM |
| | SIGLEC-10 | NSLQTEDTAIYFCTLRIKDWGPGTMVTVSSGGGGSGGGGSGGGGSDIVMTQ |
| | TM- SIGLEC- | TPPSLSVALGQSVSISCKSSQSLVASDENTYLNWLLQSPGRSPKRLIYQVSKL |
| | 10 iCAR | DSGVPDRFSGSGSEKDFTLKISRVEAEDLGVYYCLQGIHLPWTFGGGTKLEL |
| | | KGKPIPNPLLGLDSTNGAATTTPAPRPPTPAPTIALQPLSLRPEACRPAAGGAV |
| | | HTRGLDFACDGAFLGIGITALLFLCLALIIMKILPKRRTQTETPRPRFSRHSTIL |
| | | DYINVVPTAGPLAQKRNQKATPNSPRTPLPPGAPSPESKKNQKKQYQLPSFPE |
| | | PKSSTQAPESQESQEELHYATLNFPGVRPRPEARMPKGTQADYAEVKFQ |
| 113 | Anti-EMCN | CAGGTGCAGCTGAAAGAGTCTGGACCTGGACTGGTGCAGCCCAGCCAAA |
| | scFv | CACTGAGCCTGACCTGTACCGTGTCCGGCTTCAGCCTGAGCAGATACGAC |
| | nucleotide | ATGCACTGGGTCCGACAGCCTCCAGGACAAGGCTTGGAATGGATGGGCG |
| | sequence with | TGATCTGGGGCAACGGCAACACACACTATCACAGCGCCCTGAAGTCCCG |
| | (G4S)3 linker | GCTGAGCATCAGCAGAGATACCAGCAAGAGCCAGGTGTTCCTGAAGATG |
| | | AACAGCCTGCAGACCGAGGACACCGCCATCTATTTCTGCACCCTGCGGAT |
| | | CAAGGATTGGGGCCCTGGCACAATGGTCACCGTTTCTAGCGGAGGCGGA |
| | | GGATCTGGTGGCGGAGGAAGTGGCGGAGGCGGTTCTGATATCGTGATGA |
| | | CCCAGACACCTCCTAGCCTGTCTGTGGCTCTGGGCCAGTCTGTGTCCATC |
| | | AGCTGCAAGAGCAGCCAGAGCCTGGTGGCCTCCGACGAGAACACCTACC |
| | | TGAATTGGCTGCTGCAGAGCCCCGGCAGAAGCCCCAAGAGACTGATCTA |
| | | CCAGGTGTCCAAGCTGGACAGCGGCGTGCCCGATAGATTTTCTGGCAGCG |
| | | GCAGCGAGAAGGACTTCACCCTGAAGATCTCCAGAGTGGAAGCCGAGGA |
| | | CCTGGGCGTGTACTACTGTCTGCAAGGCATCCATCTGCCTTGGACCTTTG |
| | | GAGGCGGCACAAAGCTGGAACTGAAA |
| 114 | anti-FLT3 | EVQLVQSGAEVKKPGSSVKVSCKASGGTFSSYAISWVRQAPGQGLEWMGGI |
| | scFv | IPIFGTANYAQKFQGRVTITADKSTSTAYMELSSLRSEDTAVYYCATFALFGF |
| | | REQAFDIWGQGTTVTVSSGGGGSGGGGSGGGGSDIQMTQSPSSLSASVGDR |
| | | VTITCRASQSISSYLNWYQQKPGKAPKLLIYAASSLQSGVPSRFSGSGSGTDF |
| | | TLTISSLQPEDLATYYCQQSYSTPFTFGPGTKVDIK |
| 115 | anti-FLT3- | METDTLLLWVLLLWVPGSTGAGGSDYKDDDDKGGSEVQLVQSGAEVKKP |
| | CD28/CD3 ξ | GSSVKVSCKASGGTFSSYAISWVRQAPGQGLEWMGGIIPIFGTANYAQKFQG |
| | aCAR (IgK | RVTITADKSTSTAYMELSSLRSEDTAVYYCATFALFGFREQAFDIWGQGTTV |
| | signal | TVSSGGGGSGGGGSGGGGSDIQMTQSPSSLSASVGDRVTITCRASQSISSYLN |
| | sequence bold; | WYQQKPGKAPKLLIYAASSLQSGVPSRFSGSGSGTDFTLTISSLQPEDLATYY |
| | AGGS-Flag | CQQSYSTPFTFGPGTKVDIKTTTPAPRPPTPAPTIALQPLSLRPEACRPAAGGA |
| | italic) | VHTRGLDFACDFWVLVVVGGVLACYSLLVTVAFIIFWVRSKRSRLLHSDYM |
| | | NMTPRRPGPTRKHYQPYAPPRDFAAYRSRVKFSRSADAPAYKQGQNQLYNE |
| | | LNLGRREEYDVLDKRRGRDPEMGGKPRRKNPQEGLYNELQKDKMAEAYSE |
| | | IGMKGERRRGKGHDGLYQGLSTATKDTYDALHMQALPPR |
| 116 | SS (IgK)-Flag- | [symbol] ATGGAAACCGACACACTGCTGCTGTGGGTGCTGCTTCTTTG |
| | anti-FLT3- | GGTGCCCGGATCTACAGGTGCCGGCGGAAGCGACTACAAGGACGACGAT |
| | CD28/ CD3 & | GACAAAGGCGGCAGCGAGGTTCAACTGGTACAAAGCGGAGCCGAGGTAA |
| | aCAR | AGAAACCAGGGAGTAGCGTCAAAGTGTCCTGCAAAGCCTCAGGCGGCAC |
| | nucleotide | ATTCAGTAGCTATGCTATTTCATGGGTACGCCAAGCACCAGGACAGGGGC |
| | sequence (with | TGGAGTGGATGGGCGGGATTATCCCCATCTTCGGTACGGCAAACTATGCA |
| | Kozak bold | CAAAAGTTCCAGGGACGAGTCACCATCACGGCTGATAAGTCCACCTCCAC |
| | italics) | CGCCTATATGGAGCTGAGTTCCCTTCGGAGCGAGGATACTGCTGTGTATT |
| | | ATTGTGCCACGTTCGCACTGTTCGGTTTTCGGGAGCAGGCGTTTGATATTT |
| | | GGGGACAAGGCACAACGGTCACGGTCAGTTCAGGCGGAGGGGGATCAGG |
| | | GGGTGGGGGGTCAGGTGGCGGTGGAAGTGACATTCAGATGACCCAGAGT |
| | | CCCTCTTCATTGAGTGCGAGCGTCGGTGATCGGGTTACGATAACCTGTAG |
| | | GGCCTCCCAAAGTATATCATCATATTTGAACTGGTACCAACAGAAACCTG |
| | | GGAAAGCGCCGAAGCTCCTTATCTATGCTGCCAGCTCTTTGCAAAGCGGT |
| | | GTGCCCTCACGGTTCTCCGGTAGTGGGTCCGGGACCGACTTCACTTTGAC |
| | | CATCAGCAGCCTTCAGCCAGAGGATCTTGCCACTTATTACTGCCAGCAAT |
| | | CTTATAGCACACCGTTTACATTCGGTCCAGGCACAAAGGTAGACATTAAG |
| | | ACCACCACACCAGCTCCTAGACCTCCAACTCCTGCTCCTACAATCGCCCT |
| | | GCAGCCACTGAGTCTGAGGCCAGAGGCTTGTAGACCTGCTGCAGGCGGA |
| | | GCCGTGCATACAAGAGGACTGGATTTCGCCTGCGACTTCTGGGTGCTCGT |
| | | GGTTGTTGGCGGAGTGCTGGCCTGTTACAGCCTGCTGGTTACCGTGGCCT |
| | | TCATCATCTTTTGGGTCCGAAGCAAGCGGAGCCGGCTGCTGCACAGCGAT |
| | | TACATGAACATGACCCCTCGGAGGCCCGGACCTACCAGAAAGCACTACC |
| | | AGCCTTACGCTCCTCCTAGAGATTTCGCCGCCTACCGGTCCAGAGTGAAG |
| | | TTCAGCAGATCCGCCGATGCTCCCGCCTATAAGCAGGGCCAGAACCAGCT |
| | | GTACAACGAGCTGAACCTGGGGAGAAGAGAAGAGTACGACGTGCTGGAC |
| | | AAGCGGAGAGGCAGAGATCCTGAAATGGGCGGCAAGCCCAGACGGAAG |
| | | AATCCTCAAGAGGGCCTGTATAATGAGCTGCAGAAAGACAAGATGGCCG |
| | | AGGCCTACAGCGAGATCGGAATGAAGGGCGAGCGCAGAAGAGGCAAGG |
| | | GACACGATGGACTGTACCAGGGACTGAGCACCGCCACCAAGGATACCTA |
| | | TGACGCCCTGCACATGCAGGCCCTGCCTCCAAGATAA |

### Results

NK cells were engineered to express activating chimeric receptors (aCARS) and inhibitory chimeric receptors (iCARs) having various inhibitory domain formats derived from different inhibitory receptors. NK cells were virally transduced with aCAR only or in combination with iCARs having the various inhibitory domains indicated.

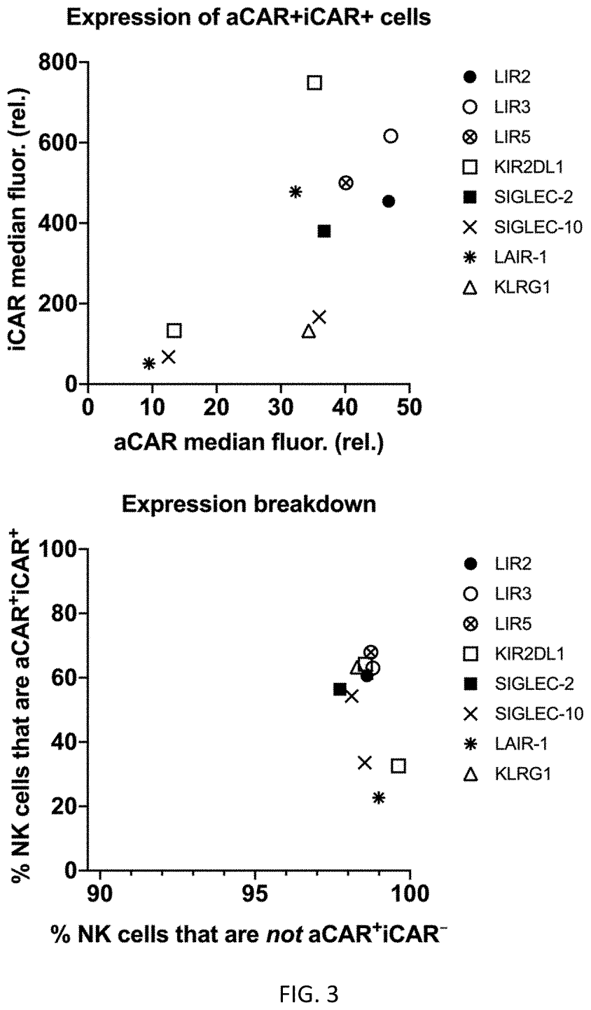

Engineered NK cells were assessed for CAR expression. As shown in FIG. 3, among aCAR+iCAR+ NK cells (top panel), anti-FLT3 aCAR expression was generally greater than 10-fold above background and anti-EMCN iCAR expression was generally greater than 100-fold. LIR family constructs demonstrated notably high expression relative to other constructs. The profile of CAR expressing populations was also assessed (bottom panel) and demonstrated the total population contained fewer than 5% aCAR+iCAR− cells and had varying percentages of aCAR+iCAR+ populations for the various iCAR formats, with KLRG1, LIR2, LIR3, LIR5, and SIGLEC-2 formats having consistently greater than 50% of cells being aCAR+iCAR+. Again, LIR family iCARs notably generally demonstrated a greater proportion of aCAR+iCAR+ cells relative to other constructs.

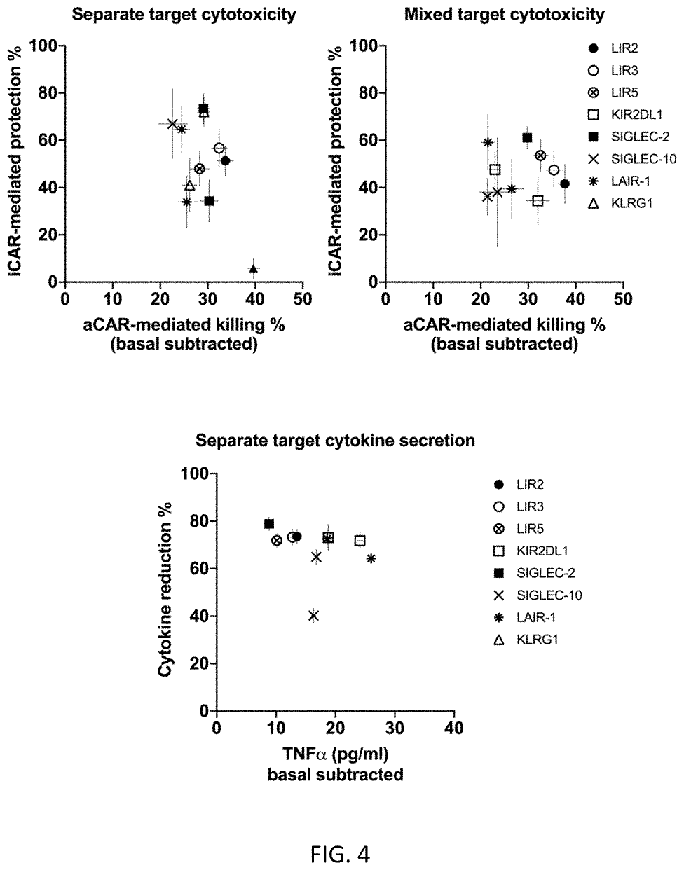

Next, anti-EMCN iCAR reduction of anti-FLT3 aCAR-induced NK cell mediated killing of target cells and NK cell cytokine production was assessed. Reduction was determined for each of the target SEM cells separately (“Separate”: aCAR antigen FLT3 only SEM cells and aCAR/iCAR antigen FLT3/EMCN co-expressing SEM cells separately) or in the context of a mixed population of target and non-target cells (“Mixed”: aCAR antigen FLT3 only SEM cells and aCAR/iCAR antigen FLT3/EMCN co-expressing SEM cells together in the same culture). As shown in FIG. 4, NK cells expressing LIR2, LIR3, LIR5, KIR2DL1, LAIR1, and SIGLEC-2 anti-EMCN iCAR formats demonstrated consistent aCAR-mediated performance in killing (top panels) and iCAR-mediated protection in both killing (top panels) and cytokine reduction (bottom panel), with SIGLEC-10 and KLRG1 constructs varying more in their performance.

The results demonstrate NK cells were successfully engineered to co-express aCARs and iCARs, successfully kill target cells and produce cytokines in the absence of an iCAR ligand in an aCAR ligand dependent manner, and various iCAR formats successfully reduced NK-mediated killing and cytokine production in an iCAR ligand dependent manner.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific embodiments have been illustrated and described, the above specification is not restrictive. It will be appreciated that various changes can be made without departing from the spirit and scope of the present disclosure(s). Many variations will become apparent to those skilled in the art upon review of this specification.

1.-15. (canceled)

16. A chimeric inhibitory receptor comprising:
(a) an extracellular protein binding domain;
(b) a transmembrane domain, wherein the transmembrane domain is operably linked to the extracellular protein binding domain; and
(c) one or more intracellular signaling domains, wherein the one or more intracellular signaling domains are operably linked to the transmembrane domain, and
wherein each of the one or more intracellular signaling domains is derived from a protein selected from the group consisting of SLAP1, SLAP2, Dok-1, Dok-2, GRB-2, CD200R, SIRPα, HAVR, GITR, PD-L1, KIR2DL2, KIR2DL3, KIR3DL2, CD94, KLRG-1, CEACAM1, LIR2, LIR3, LIR5, SIGLEC-2, and SIGLEC-10, and
wherein at least one of the one or more intracellular signaling domains is capable of preventing, attenuating, or inhibiting activation of an immunomodulatory cell.

17. The chimeric inhibitory receptor of claim 16, wherein:
(a) the transmembrane domain and one of the one or more intracellular signaling domains are derived from the same protein, optionally wherein the transmembrane domain further comprises at least a portion of an extracellular domain of the same protein; or
(b) the transmembrane domain is derived from a first protein and each of the one or more intracellular signaling domains is derived from a second protein that is distinct from the first protein.

18. The chimeric inhibitory receptor of claim 16, wherein:
(a) one of the one or more intracellular signaling domains is derived from SLAP1, optionally wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to PAPAERPLPNPEGLDSDFLAVLSDYPSPDISPPIFRRGEKLRVISDEGGWWKAISLSTGRE SYIPGICVARVYHGWLFEGLGRDKAEELLQLPDTKVGSFMIRESETKKGFYSLSVRHRQ VKHYRIFRLPNNVVYYISPRLTFQCLEDLVNHYSEVADGLCCVLTTPCLTQSTAAPAVRA SSSPVTLRQKTVDWRRVSRLQEDPEGTENPLGVDESLFSYGLRESIASYLSLTSEDNTSF DRKKKSISLMYGGSKRKSSFFSSPPYFED (SEQ ID NO: 4), or wherein the intracellular signaling domain comprises the amino acid sequence of PAPAERPLPNPEGLDSDFLAVLSDYPSPDISPPIFRRGEKLRVISDEGGWWKAISLSTGRE SYIPGICVARVYHGWLFEGLGRDKAEELLQLPDTKVGSFMIRESETKKGFYSLSVRHRQ VKHYRIFRLPNNVVYYISPRLTFQCLEDLVNHYSEVADGLCCVLTTPCLTQSTAAPAVRA SSSPVTLRQKTVDWRRVSRLQEDPEGTENPLGVDESLFSYGLRESIASYLSLTSEDNTSF DRKKKSISLMYGGSKRKSSFFSSPPYFED (SEQ ID NO: 4); or
(b) one of the one or more intracellular signaling domains is derived from SLAP1, optionally wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to PAPAERPLPNPEGLDSDFLAVLSDYPSPDISPPIFRRGEKLRVISDEGGWWKAISLSTGRE SYIPGICVARVYHGWLFEGLGRDKAEELLQLPDTKVGSFMIRESETKKGFYSLSVRHRQ VKHYRIFRLPNNVVYYISPRLTFQCLEDLVNHYSEVADGLCCVLTTPCLTQSTAAPAVRA SSSPVTLRQKTVDWRRVSRLQEDPEGTENPLGVDESLFSYGLRESIASYLSLTSEDNTSF (SEQ ID NO: 5), or wherein the intracellular signaling domain comprises the amino acid sequence of PAPAERPLPNPEGLDSDFLAVLSDYPSPDISPPIFRRGEKLRVISDEGGWWKAISLSTGRE SYIPGICVARVYHGWLFEGLGRDKAEELLQLPDTKVGSFMIRESETKKGFYSLSVRHRQ VKHYRIFRLPNNVVYYISPRLTFQCLEDLVNHYSEVADGLCCVLTTPCLTQSTAAPAVRA SSSPVTLRQKTVDWRRVSRLQEDPEGTENPLGVDESLFSYGLRESIASYLSLTSEDNTSF (SEQ ID NO: 5); or
(c) one of the one or more intracellular signaling domains is derived from SLAP2, optionally wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RKSLPSPSLSSSVQGQGPVTMEAERSKATAVALGSFPAGGPAELSLRLGEPLTIVSEDGD WWTVLSEVSGREYNIPSVHVAKVSHGWLYEGLSREKAEELLLLPGNPGGAFLIRESQTR RGSYSLSVRLSRPASWDRIRHYRIHCLDNGWLYISPRLTFPSLQALVDHYSELADDICCL LKEPCVLQRAGPLPGKDIPLPVTVQRTPLNWKELDSSLLFSEAATGEESLLSEGLRESLSF YISLNDEAVSLDDA (SEQ ID NO: 6), or wherein the intracellular signaling domain comprises the amino acid sequence of RKSLPSPSLSSSVQGQGPVTMEAERSKATAVALGSFPAGGPAELSLRLGEPLTIVSEDGD WWTVLSEVSGREYNIPSVHVAKVSHGWLYEGLSREKAEELLLLPGNPGGAFLIRESQTR RGSYSLSVRLSRPASWDRIRHYRIHCLDNGWLYISPRLTFPSLQALVDHYSELADDICCL LKEPCVLQRAGPLPGKDIPLPVTVQRTPLNWKELDSSLLFSEAATGEESLLSEGLRESLSF YISLNDEAVSLDDA (SEQ ID NO: 6); or
(d) one of the one or more intracellular signaling domains is derived from KLRG-1, optionally wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to MTDSVIYSMLELPTATQAQNDYGPQQKSSSSRPSCSCLGSG (SEQ ID NO: 61), or wherein the intracellular signaling domain comprises the amino acid sequence of MTDSVIYSMLELPTATQAQNDYGPQQKSSSSRPSCSCLGSG (SEQ ID NO: 61); or
(e) one of the one or more intracellular signaling domains is derived from LIR2, optionally wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LRHRRQGKHWTSTQRKADFQHPAGAVGPEPTDRGLQWRSSPAADAQEENLYAAVKDT QPEDGVEMDTRAAASEAPQDVTYAQLHSLTLRRKATEPPPSQEREPPAEPSIYATLATH (SEQ ID NO: 63), or wherein the intracellular signaling domain comprises the amino acid sequence of LRHRRQGKHWTSTQRKADFQHPAGAVGPEPTDRGLQWRSSPAADAQEENLYAAVKDT QPEDGVEMDTRAAASEAPQDVTYAQLHSLTLRRKATEPPPSQEREPPAEPSIYATLATH (SEQ ID NO: 63); or
(f) one of the one or more intracellular signaling domains is derived from LIR3, optionally wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RRQRHSKHRTSDQRKTDFQRPAGAAETEPKDRGLLRRSSPAADVQEENLYAAVKDTQS EDRVELDSQSPHDEDPQAVTYAPVKHSSPRREMASPPSSLSGEFLDTKDRQVEEDRQMD TEAAASEASQDVTYAQLHSLTLRRKATEPPPSQEGEPPAEPSIYATLAIH (SEQ ID NO: 64), or wherein the intracellular signaling domain comprises the amino acid sequence of RRQRHSKHRTSDQRKTDFQRPAGAAETEPKDRGLLRRS SPAADVQEENLYAAVKDTQS EDRVELDSQSPHDEDPQAVTYAPVKHSSPRREMASPPSSLSGEFLDTKDRQVEEDRQMD TEAAASEASQDVTYAQLHSLTLRRKATEPPPSQEGEPPAEPSIYATLAIH (SEQ ID NO: 64); or
(g) one of the one or more intracellular signaling domains is derived from LIR5, optionally wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to QHWRQGKHRTLAQRQADFQRPPGAAEPEPKDGGLQRRSSPAADVQGENFCAAVKNTQ PEDGVEMDTRQSPHDEDPQAVTYAKVKHSRPRREMASPPSPLSGEFLDTKDRQAEEDR QMDTEAAASEAPQDVTYAQLHSFTLRQKATEPPPSQEGASPAEPSVYATLAIH (SEQ ID NO: 65), or wherein the intracellular signaling domain comprises the amino acid sequence of QHWRQGKHRTLAQRQADFQRPPGAAEPEPKDGGLQRRSSPAADVQGENFCAAVKNTQ PEDGVEMDTRQSPHDEDPQAVTYAKVKHSRPRREMASPPSPLSGEFLDTKDRQAEEDR QMDTEAAASEAPQDVTYAQLHSFTLRQKATEPPPSQEGASPAEPSVYATLAIH (SEQ ID NO: 65); or
(h) one of the one or more intracellular signaling domains is derived from SIGLEC-2, optionally wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to KLQRRWKRTQSQQGLQENSSGQSFFVRNKKVRRAPLSEGPHSLGCYNPMMEDGISYTT LRFPEMNIPRTGDAESSEMQRPPPDCDDTVTYSALHKRQVGDYENVIPDFPEDEGIHYSE LIQFGVGERPQAQENVDYVILKH (SEQ ID NO: 66), or wherein the intracellular signaling domain comprises the amino acid sequence of KLQRRWKRTQSQQGLQENSSGQSFFVRNKKVRRAPLSEGPHSLGCYNPMMEDGISYTT LRFPEMNIPRTGDAESSEMQRPPPDCDDTVTYSALHKRQVGDYENVIPDFPEDEGIHYSE LIQFGVGERPQAQENVDYVILKH (SEQ ID NO: 66); or
(i) one of the one or more intracellular signaling domains is derived from SIGLEC-10, optionally wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to KILPKRRTQTETPRPRFSRHSTILDYINVVPTAGPLAQKRNQKATPNSPRTPLPPGAPSPES KKNQKKQYQLPSFPEPKSSTQAPESQESQEELHYATLNFPGVRPRPEARMPKGTQADYA EVKFQ (SEQ ID NO: 67), or wherein the intracellular signaling domain comprises the amino acid sequence of KILPKRRTQTETPRPRFSRHSTILDYINVVPTAGPLAQKRNQKATPNSPRTPLPPGAPSPES KKNQKKQYQLPSFPEPKSSTQAPESQESQEELHYATLNFPGVRPRPEARMPKGTQADYA EVKFQ (SEQ ID NO: 67), or
(j) one of the one or more intracellular signaling domains is derived from SIRPα, optionally wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RIRQKKAQGSTSSTRLHEPEKNAREITQDTNDITYADLNLPKGKKPAPQAAEPNNHTEY ASIQTSPQPASEDTLTYADLDMVHLNRTPKQPAPKPEPSFSEYASVQVPRK (SEQ ID NO: 8), or wherein the intracellular signaling domain comprises the amino acid sequence of
(SEQ ID NO: 8)
RIRQKKAQGSTSSTRLHEPEKNAREITQDTNDITYADLNLPKGKKPAPQA
AEPNNHTEYASIQTSPQPASEDTLTYADLDMVHLNRTPKQPAPKPEPSFS
EYASVQVPRK.

19. The chimeric inhibitory receptor of claim 16, wherein:
(a) the transmembrane domain is derived from a protein selected from the group consisting of: CD8, CD28, CD3, CD4, 4-IBB, OX40, ICOS, 2B4, CD25, CD7, LAX, LAT, LAIR′, GRB-2, Dok-1, Dok-2, SLAP1, SLAP2, CD200R, SIRPα, HAVR, GITR, PD-L1, KIR2DL1, KIR2DL2, KIR2DL3, KIR3DL2, CD94, KLRG-1, CEACAM1, LIR2, LIR3, LIR5, SIGLEC-2, and SIGLEC-10; or
(b) the transmembrane domain is derived from CD28, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to FWVLVVVGGVLACYSLLVTVAFIIFWV (SEQ ID NO: 20), or wherein the transmembrane domain comprises the amino acid sequence of FWVLVVVGGVLACYSLLVTVAFIIFWV (SEQ ID NO: 20); or
(c) the transmembrane domain is derived from KIR2DL1, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to ILIGTSVVIILFILLFFLL (SEQ ID NO: 76), or wherein the transmembrane domain comprises the amino acid sequence of ILIGTSVVIILFILLFFLL (SEQ ID NO: 76); or
(d) the transmembrane domain is derived from KLRG-1, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VAIALGLLTAVLLSVLLYQWI (SEQ ID NO: 78), or wherein the transmembrane domain comprises the amino acid sequence of VAIALGLLTAVLLSVLLYQWI (SEQ ID NO: 78); or
(e) the transmembrane domain is derived from LAIR1, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to ILIGVSVVFLFCLLLLVLFCL (SEQ ID NO: 79), or wherein the transmembrane domain comprises the amino acid sequence of ILIGVSVVFLFCLLLLVLFCL (SEQ ID NO: 79); or
(f) the transmembrane domain is derived from LIR2, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VIGILVAVVLLLLLLLLLFLI (SEQ ID NO: 80), or wherein the transmembrane domain comprises the amino acid sequence of VIGILVAVVLLLLLLLLLFLI (SEQ ID NO: 80); or
(g) the transmembrane domain is derived from LIR3, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VLIGVSVAFVLLLFLLLFLLL (SEQ ID NO: 81), or wherein the transmembrane domain comprises the amino acid sequence of VLIGVSVAFVLLLFLLLFLLL (SEQ ID NO: 81); or
(h) the transmembrane domain is derived from LIR5, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VLIGVLVVSILLLSLLLFLLL (SEQ ID NO: 82), or wherein the transmembrane domain comprises the amino acid sequence of VLIGVLVVSILLLSLLLFLLL (SEQ ID NO: 82); or
(i) the transmembrane domain is derived from SIGLEC-2, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VAVGLGSCLAILILAICGL (SEQ ID NO: 83), or wherein the transmembrane domain comprises the amino acid sequence of VAVGLGSCLAILILAICGL (SEQ ID NO: 83); or
(j) the transmembrane domain is derived from SIGLEC-10, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to GAFLGIGITALLFLCLALIIM (SEQ ID NO: 84), or wherein the transmembrane domain comprises the amino acid sequence of GAFLGIGITALLFLCLALIIM (SEQ ID NO: 84); or
(k) the transmembrane domain is derived from SIRPα, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to IVVGVVCTLLVALLMAALYLV (SEQ ID NO: 21), or wherein the transmembrane domain comprises the amino acid sequence of
(SEQ ID NO: 21)
IVVGVVCTLLVALLMAALYLV.

20. The chimeric inhibitory receptor of claim 16, wherein:
(a) the chimeric inhibitory receptor comprises a first intracellular signaling domain derived from KIR2DL1 and a second intracellular signaling domain derived from LIR2; or
(b) the chimeric inhibitory receptor comprises a first intracellular signaling domain derived from KIR2DL1 and a second intracellular signaling domain derived from LIR3; or
(c) the chimeric inhibitory receptor comprises a first intracellular signaling domain derived from KIR2DL1 and a second intracellular signaling domain derived from LIR5; or
(d) the chimeric inhibitory receptor comprises a first intracellular signaling domain derived from LIR2 and a second intracellular signaling domain derived from KIR2DL1; or
(e) the chimeric inhibitory receptor comprises a first intracellular signaling domain derived from LIR3 and a second intracellular signaling domain derived from KIR2DL1; or
(f) the chimeric inhibitory receptor comprises a first intracellular signaling domain derived from LIR5 and a second intracellular signaling domain derived from KIR2DL1; or
(g) the chimeric inhibitor receptor comprises an intracellular signaling domain derived from SIRPα and a transmembrane domain derived from SIRPα, optionally wherein the intracellular signaling domain comprises the amino acid sequence RIRQKKAQGSTSSTRLHEPEKNAREITQDTNDITYADLNLPKGKKPAPQAAEPNNHTEY ASIQTSPQPASEDTLTYADLDMVHLNRTPKQPAPKPEPSFSEYASVQVPRK (SEQ ID NO: 8) and wherein the transmembrane domain comprises the amino acid sequence
(SEQ ID NO: 21)
IVVGVVCTLLVALLMAALYLV.

21. The chimeric inhibitory receptor of claim 16, wherein:
(a) the protein binding domain binds a protein that is not expressed on the target tumor, or the protein binding domain binds a protein that is expressed on a non-tumor cell, optionally the non-tumor cell is derived from a tissue selected from the group consisting of brain, neuronal tissue, endocrine, endothelial, bone, bone marrow, immune system, muscle, lung, liver, gallbladder, pancreas, gastrointestinal tract, kidney, urinary bladder, male reproductive organs, female reproductive organs, adipose, soft tissue, and skin; and
(b) the extracellular protein binding domain comprises a ligand-binding domain, or the extracellular protein binding domain comprises a receptor-binding domain, or the extracellular protein binding domain comprises an antigen-binding domain, optionally wherein when the extracellular protein binding domain comprises an antigen-binding domain, wherein the antigen-binding domain comprises an antibody, an antigen-binding fragment of an antibody, a F(ab) fragment, a F(ab′) fragment, a single chain variable fragment (scFv), or a single-domain antibody (sdAb), and optionally wherein when the antigen-binding domain comprises an scFv, the scFv comprises a heavy chain variable domain (VH) and a light chain variable domain (VL) and the VH and VL are separated by a peptide linker, and optionally wherein the peptide linker comprises an amino acid sequence selected from the group consisting of: GGS (SEQ ID NO: 23), GGSGGS (SEQ ID NO: 24), GGSGGSGGS (SEQ ID NO: 25), GGSGGSGGSGGS (SEQ ID NO: 26), GGSGGSGGSGGSGGS (SEQ ID NO: 27), GGGS (SEQ ID NO: 28), GGGSGGGS (SEQ ID NO: 29), GGGSGGGSGGGS (SEQ ID NO: 30), GGGSGGGSGGGSGGGS (SEQ ID NO: 31), GGGSGGGSGGGSGGGSGGGS (SEQ ID NO: 32), GGGGS (SEQ ID NO: 33), GGGGSGGGGS (SEQ ID NO: 34), GGGGSGGGGSGGGGS (SEQ ID NO: 35), GGGGSGGGGSGGGGSGGGGS (SEQ ID NO: 36), GGGGSGGGGSGGGGSGGGGSGGGGS (SEQ ID NO: 37), and TTTPAPRPPTPAPTIALQPLSLRPEACRPAAGGAVHTRGLDFACDQTTPGERSSLPAFYPG TSGSCSGCGSLSLP (SEQ ID NO: 94).

22. The chimeric inhibitory receptor of claim 16, wherein the chimeric inhibitory receptor further comprises a spacer region positioned between the extracellular protein binding domain and the transmembrane domain and operably linked, or physically linked, to each of the extracellular protein binding domain and the transmembrane domain,
optionally wherein the chimeric inhibitory receptor further comprises an intracellular spacer region positioned between the transmembrane domain and one of the one or more intracellular signaling domains and operably linked, or physically linked, to each of the transmembrane domain and the one of the one or more intracellular signaling domains,
optionally wherein the spacer region is derived from a protein selected from the group consisting of: CD8α, CD4, CD7, CD28, IgG1, IgG4, FcγRIIIα, LNGFR, and PDGFR, or wherein the spacer region comprises an amino acid sequence selected from the group consisting of:
(SEQ ID NO: 45)
TTTPAPRPPTPAPTIALQPLSLRPEACRPAAGGAVHTRGLDFACD,
(SEQ ID NO: 39)
AAAIEVMYPPPYLDNEKSNGTIIHVKGKHLCPSPLFPGPSKP,
(SEQ ID NO: 40)
ESKYGPPCPSCP,
(SEQ ID NO: 41)
ESKYGPPAPSAP,
(SEQ ID NO: 42)
ESKYGPPCPPCP,
(SEQ ID NO: 43)
EPKSCDKTHTCP,
(SEQ ID NO: 44)
AAAFVPVFLPAKPTTTPAPRPPTPAPTIASQPLSLRPEACRPAAGGAV
HTRGLDFACDIYIWAPLAGTCGVLLLSLVITLYCNHRN,
(SEQ ID NO: 46)
ACPTGLYTHSGECCKACNLGEGVAQPCGANQTVCEPCLDSVTFSDVVS
ATEPCKPCTECVGLQSMSAPCVEADDAVCRCAYGYYQDETTGRCEACR
VCEAGSGLVFSCQDKQNTVCEECPDGTYSDEADAEC,
(SEQ ID NO: 47)
ACPTGLYTHSGECCKACNLGEGVAQPCGANQTVC,
and
(SEQ ID NO: 48)
AVGQDTQEVIVVPHSLPFKV.

23. The chimeric inhibitory receptor of claim 16, wherein the immunomodulatory cell expresses a tumor-targeting chimeric receptor, and wherein the tumor-targeting chimeric receptor is a chimeric antigen receptor (CAR) or an engineered T cell receptor (TCR).

24. The chimeric inhibitory receptor of claim 16, wherein the immunomodulatory cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell.

25. An engineered nucleic acid encoding the chimeric inhibitory receptor of claim 16.

26. An expression vector comprising the engineered nucleic acid of claim 25.

27. An isolated immunomodulatory cell comprising the chimeric inhibitory receptor of claim 16, optionally wherein the cell further comprises a tumor-targeting chimeric receptor expressed on the surface of the cell, and optionally wherein upon binding of the protein to the chimeric inhibitory receptor, the chimeric inhibitory receptor prevents, attenuates, or inhibits activation of the tumor-targeting chimeric receptor relative to an otherwise identical cell lacking a chimeric inhibitory receptor.

28. A composition comprising:
(a) the isolated immunomodulatory cell of claim 27; and
(b) a pharmaceutically acceptable carrier, pharmaceutically acceptable excipient, or a combination thereof.

29. A method of preventing, attenuating, or inhibiting a cell-mediated immune response of an immunomodulatory cell, comprising:
engineering the immunomodulatory cell to express a chimeric inhibitory receptor comprising:
(a) an extracellular protein binding domain;
(b) a transmembrane domain, wherein the transmembrane domain is operably linked to the extracellular protein binding domain; and
(c) one or more intracellular signaling domains, wherein the one or more intracellular signaling domains are operably linked to the transmembrane domain, and
wherein each of the one or more intracellular signaling domains is derived from a protein selected from the group consisting of SLAP1, SLAP2, Dok-1, Dok-2, GRB-2, CD200R, SIRPα, HAVR, GITR, PD-L1, KIR2DL2, KIR2DL3, KIR3DL2, CD94, KLRG-1, CEACAM1, LIR2, LIR3, LIR5, SIGLEC-2, and SIGLEC-10,
wherein upon binding of a cognate antigen to the chimeric inhibitory receptor, the intracellular signaling domain prevents, attenuates, or inhibits activation of the immunomodulatory cell.

30. A method of preventing, attenuating, or inhibiting an immune response of an engineered immunomodulatory cell to a healthy cell of a subject, comprising:
providing an engineered immunomodulatory cell to the subject, the engineered immunomodulatory cell comprising:
(a) an extracellular protein binding domain;
(b) a transmembrane domain, wherein the transmembrane domain is operably linked to the extracellular protein binding domain; and
(c) one or more intracellular signaling domains, wherein the one or more intracellular signaling domains are operably linked to the transmembrane domain, and
wherein each of the one or more intracellular signaling domains is derived from a protein selected from the group consisting of SLAP1, SLAP2, Dok-1, Dok-2, GRB-2, CD200R, SIRPα, HAVR, GITR, PD-L1, KIR2DL2, KIR2DL3, KIR3DL2, CD94, KLRG-1, CEACAM1, LIR2, LIR3, LIR5, SIGLEC-2, and SIGLEC-10,
wherein upon binding of the chimeric inhibitory receptor to its cognate antigen, the intracellular signaling domain prevents, attenuates, or inhibits activation of the engineered immunomodulatory cell to a healthy cell of the subject.

31. The method of claim 30, wherein the engineered immunomodulatory cell further comprises a tumor-targeting chimeric receptor, optionally wherein the tumor-targeting chimeric receptor is a chimeric antigen receptor (CAR) or an engineered T cell receptor, and optionally wherein the CAR binds one or more antigens expressed on the surface of a tumor cell.

32. The chimeric inhibitory receptor of claim 16, wherein the one or more intracellular signaling domains is a SIRPα intracellular domain.

33. The chimeric inhibitory receptor of claim 16, wherein the transmembrane domain is a SIRPα transmembrane domain.

34. The chimeric inhibitory receptor of claim 16, wherein the transmembrane domain is derived from SIRPα, the one or more intracellular signaling domains is derived from SIRPα, and the chimeric inhibitory receptor further comprises a spacer region derived from CD8α positioned between the extracellular protein binding domain and the transmembrane domain.

35. The method of claim 29, wherein the engineering further comprises engineering the cell to express a tumor targeting chimeric receptor, optionally wherein the tumor-targeting chimeric receptor is a chimeric antigen receptor (CAR) or an engineered T cell receptor, and optionally wherein the CAR binds one or more antigens expressed on the surface of a tumor cell.


SEQUENCE LISTING (116 sequences)

SEQ ID NO: 1 - protein, 217 aa, synthetic construct [sequence omitted]

SEQ ID NO: 2 - protein, 481 aa, synthetic construct [sequence omitted]

SEQ ID NO: 3 - protein, 412 aa, synthetic construct
MGDGAVKQGFLYLQQQQTFGKKWRRFGASLYGGSDCALARLELQEGPEKPRRCEAARKVIRLSDCLRVAEAGGEASSPRDTSAFFLETKERLYLLAAPAAERGDWVQAICLLAFPGQRKELSGPEGKQSRPCMEENELYSSAVTVGPHKEFAVTMRPTEASERCHLRGSYTLRAGESALELWGGPEPGTQLYDWPYRFLRRFGRDKVTFSFEAGRRCVSGEGNFEFETRQGNEIFLALEEAISAQKNAAPATPQPQPATIPASLPRPDSPYSRPHDSLPPPSPTTPVPAPRPRGQEGEYAVPFDAVARSLGKNFRGILAVPPQLLADPLYDSIEETLPPRPDHIYDEPEGVAALSLYDSPQEPRGEAWRRQATADRDPAGLQHVQPAGQDFSASGWQPGTEYDNVVLKKGPK

SEQ ID NO: 4 - protein, 268 aa, synthetic construct
PAPAERPLPNPEGLDSDFLAVLSDYPSPDISPPIFRRGEKLRVISDEGGWWKAISLSTGRESYIPGICVARVYHGWLFEGLGRDKAEELLQLPDTKVGSFMIRESETKKGFYSLSVRHRQVKHYRIFRLPNNWYYISPRLTFQCLEDLVNHYSEVADGLCCVLTTPCLTQSTAAPAVRASSSPVTLRQKTVDWRRVSRLQEDPEGTENPLGVDESLFSYGLRESIASYLSLTSEDNTSFDRKKKSISLMYGGSKRKSSFFSSPPYFED

SEQ ID NO: 5 - protein, 239 aa, synthetic construct
PAPAERPLPNPEGLDSDFLAVLSDYPSPDISPPIFRRGEKLRVISDEGGWWKAISLSTGRESYIPGICVARVYHGWLFEGLGRDKAEELLQLPDTKVGSFMIRESETKKGFYSLSVRHRQVKHYRIFRLPNNWYYISPRLTFQCLEDLVNHYSEVADGLCCVLTTPCLTQSTAAPAVRASSSPVTLRQKTVDWRRVSRLQEDPEGTENPLGVDESLFSYGLRESIASYLSLTSEDNTSF

SEQ ID NO: 6 - protein, 254 aa, synthetic construct
RKSLPSPSLSSSVQGQGPVTMEAERSKATAVALGSFPAGGPAELSLRLGEPLTIVSEDGDWWTVLSEVSGREYNIPSVHVAKVSHGWLYEGLSREKAEELLLLPGNPGGAFLIRESQTRRGSYSLSVRLSRPASWDRIRHYRIHCLDNGWLYISPRLTFPSLQALVDHYSELADDICCLLKEPCVLQRAGPLPGKDIPLPVTVQRTPLNWKELDSSLLFSEAATGEESLLSEGLRESLSFYISLNDEAVSLDDA

SEQ ID NO: 7 - protein, 61 aa, synthetic construct
KVNGCRKYKLNKTESTPVVEEDEMQPYASYTEKNNPLYDTTNKVKASEALQSEVDTDLHTL

SEQ ID NO: 8 - protein, 110 aa, synthetic construct
RIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 9 - DNA, 651 bp, synthetic construct
  source 1..651: mol_type = other DNA
atggaagccattgccaaatacgacttcaaggccaccgccgacgacgagctgagcttcaagagaggcgacatcctgaaggtgctgaacgaggaatgcgaccagaactggtacaaggccgagctgaacggcaaggacggcttcatccccaagaactacatcgagatgaagccccatccatggttcttcggcaagatccccagagccaaggccgaagagatgctgagcaagcagagacacgacggcgcctttctgatccgggaatctgaatctgcccctggcgacttcagcctgagcgtgaagttcggcaacgacgtgcagcacttcaaggtcctgagagatggcgccggaaagtacttcctgtgggtcgtgaagtttaacagcctgaacgagctggtggactaccacagatccaccagcgtgtcccggaaccagcagatcttcctgcgggacatcgaacaggtgccacagcagccaacatacgtgcaggccctgttcgacttcgaccctcaagaggatggcgagctgggctttagacggggcgatttcatccacgtgatggacaacagcgaccccaactggtggaagggcgcttgtcatggacagaccggcatgttccccagaaactacgtgacccctgtgaaccggaacgtg

SEQ ID NO: 10 - DNA, 1443 bp, synthetic construct
  source 1..1443: mol_type = other DNA
atggatggcgccgtgatggaaggccctctgtttctccagagccagagattcggcaccaagcggtggcggaaaacatgggccgttctgtaccctgcctctcctcatggcgtggcccggctggaatttttcgatcacaagggctctagcagcggcggaggcagaggatctagtagacggctggactgcaaagtgatccggctggccgagtgtgtgtctgtggctcctgtgaccgtggaaacccctcctgaacctggcgccacagccttcagactggatacagcccagagaagccatctgctggccgccgatgctccttcttctgctgcttgggtgcagaccctgtgccggaacgcttttcctaaaggcagctggacactggcccctaccgacaatcctcctaagctgagcgccctggaaatgctggaaaacagcctgtacagccccacctgggagggctctcagttttgggtcaccgtgcagagaacagaggccgccgaaagatgtggcctgcacggctcttatgtgctgagagtggaagccgagagactgaccctgctgacagtgggagcccagtctcagatcctggaacctctgctgagctggccctacacactgctgagaagatacggccgggacaaagtgatgttcagcttcgaggccggcagaagatgtccttctggccctggcacattcacatttcagacagcccagggcaacgacatcttccaggctgtggaaaccgccatccacagacagaaggcccagggaaaagccggccagggacacgatgttctgagagccgattctcacgagggcgaagtggccgagggaaagcttccttctccacctggacctcaagagctgctggatagccctcctgctctgtatgccgagcctctggacagcctgagaatcgccccttgtccaagccaggactctctgtacagcgatcccctggatagcacatctgcccaagctggcgaaggcgtgcagaggaagaagcccctgtactgggatctgtacgagcacgctcagcagcaactgctgaaggccaagctgacagaccccaaagaggaccccatctacgacgagcctgaaggacttgctccagtgccacctcagggcctgtacgatctgcctagagagcctaaagacgcctggtggtgtcaggccagagtgaaagaggaaggctacgagctgccttacaaccccgccaccgatgattatgccgtgcctcctccaagaagcaccaaaccactgctggccccaaagcctcagggacctgcttttcctgagcctggaacagccacaggcagcggcatcaagagccacaatagcgccctgtatagccaggtgcagaaaagcggcgccagcggctcttgggattgtggacttagcagagtgggcaccgacaagaccggcgtgaagtctgagggaagcaca

SEQ ID NO: 11 - DNA, 1236 bp, synthetic construct
  source 1..1236: mol_type = other DNA
atgggagatggcgccgtgaagcagggctttctgtatctccagcagcagcagaccttcggcaagaagtggcggagatttggcgcctctctgtacggcggctctgattgtgctctggcccgactggaactgcaagagggacctgagaagcccagaagatgcgaggccgccagaaaagtgatccggctgagcgattgtctgagagtggctgaagcaggcggcgaagccagctctcctagagataccagcgcattcttcctggaaacaaaagagcggctgtacctgctggccgctcctgctgctgaaagaggcgattgggtccaagccatctgcctgctggcttttcccggccagagaaaagagctgtctggccctgagggcaagcagagcaggccttgcatggaagagaacgagctgtactccagcgccgtgacagtgggccctcacaaagaatttgccgtgaccatgaggcccaccgaggccagcgaaagatgtcacctgagaggcagctacaccctgagagccggcgaatctgctctggaactttggggaggacctgagcctggcacacagctgtacgactggccctacagattcctgcggagattcggccgggacaaagtgaccttcagctttgaggctggccgcagatgtgtgtccggcgagggcaatttcgagttcgagacaagacagggcaacgagatcttcctggctctggaagaggccatcagcgcccagaaaaatgctgcccctgctacacctcagcctcagcctgctacaatccctgcctctctgcccagacctgacagcccttatagcagaccccacgactctctgcctccaccttctccaacaacacccgtgcctgctcctagacctagaggacaagagggcgagtacgccgtgccttttgatgccgtggctagaagcctgggcaagaacttcagaggcatcctggctgtgcctccacagctgctggctgaccctctgtacgacagcatcgaggaaaccctgcctccaagacctgaccacatctacgacgagcctgaaggcgtggcagccctgtctctgtatgactcccctcaagagcctagaggcgaagcctggcgtagacaggctaccgccgatagagatcctgccggactgcaacatgtgcagccagccggccaggatttttctgcctctggatggcagccaggcaccgagtacgataacgtggtgctgaagaagggccccaag

SEQ ID NO: 12 - DNA, 804 bp, synthetic construct
  source 1..804: mol_type = other DNA
ccagccccagcggagcgaccgctgccaaaccctgaagggctcgacagtgactttttggctgtcctctccgactatcctagtcccgatatcagccccccgatattcaggcgcggtgaaaaactccgagtcatcagcgatgaggggggttggtggaaagccatcagcctgagtaccggacgagagtcatacattcctggaatatgtgtagcgagggtgtaccacggttggctgttcgagggtctgggaagagataaggccgaggaacttctccaactcccagatacaaaagtcggtagttttatgattcgggaaagtgaaactaaaaaggggttctatagcctctcagttcggcataggcaggtcaagcattatcggatatttcgcttgcctaacaactggtactacataagtccccgactcacattccaatgcctggaggacctcgtgaatcactattcagaagttgcagatggcctctgctgcgtactcacgacaccctgccttacccagagtacagcggccccggctgttcgggcatcttccagcccagtaacactcaggcaaaaaactgtggattggcgcagagtctcacgccttcaggaagatcctgagggtacggaaaatccgctcggtgtggacgagtcactgttctcctatggcttgagggaatctatagcgtcttacctttccttgacgtctgaagataatacgtctttcgatcgcaaaaaaaaatcaatatctcttatgtatggcggaagcaaaaggaaatcatcattcttttcctctccaccatatttcgaagat

SEQ ID NO: 13 - DNA, 717 bp, synthetic construct
  source 1..717: mol_type = other DNA
ccagccccagcggagcgaccgctgccaaaccctgaagggctcgacagtgactttttggctgtcctctccgactatcctagtcccgatatcagccccccgatattcaggcgcggtgaaaaactccgagtcatcagcgatgaggggggttggtggaaagccatcagcctgagtaccggacgagagtcatacattcctggaatatgtgtagcgagggtgtaccacggttggctgttcgagggtctgggaagagataaggccgaggaacttctccaactcccagatacaaaagtcggtagttttatgattcgggaaagtgaaactaaaaaggggttctatagcctctcagttcggcataggcaggtcaagcattatcggatatttcgcttgcctaacaactggtactacataagtccccgactcacattccaatgcctggaggacctcgtgaatcactattcagaagttgcagatggcctctgctgcgtactcacgacaccctgccttacccagagtacagcggccccggctgttcgggcatcttccagcccagtaacactcaggcaaaaaactgtggattggcgcagagtctcacgccttcaggaagatcctgagggtacggaaaatccgctcggtgtggacgagtcactgttctcctatggcttgagggaatctatagcgtcttacctttccttgacgtctgaagataatacgtctttc

SEQ ID NO: 14 - DNA, 762 bp, synthetic construct
  source 1..762: mol_type = other DNA
agaaaatccctccccagtccaagcctgtctagtagcgttcagggtcagggcccagttactatggaagcggaacgatccaaggctaccgcagttgctctgggttcattcccggctggagggccagcggaactctccttgcgcctgggtgagccacttaccatcgtttctgaggacggagattggtggacggttctttctgaagtatctgggagagaatacaacataccgtcagttcatgtcgcaaaagtttcacacggttggctttacgagggactcagcagggaaaaagcggaggaattgttgcttttgcccggcaatcctggcggagcatttttgataagagagagtcagacccggagaggcagttattccctgtcagttagactcagcagacctgcgagttgggataggattcggcactacaggattcactgccttgataacggctggctgtatatatctcctcgcctgacattccctagcctccaagcgttggttgatcattactctgaactggcagacgatatctgctgcctgctcaaggaaccgtgcgtcctccagagggcaggcccacttcctggcaaggatattccacttccagtcacggttcaaagaacccccctcaattggaaggagctggatagctctctcctcttttccgaggccgctacaggtgaagaatctctgttgtctgaaggattgagagagagtctctccttctacatctctctgaatgatgaagcagtgtcattggacgacgca

SEQ ID NO: 15 - DNA, 183 bp, synthetic construct
  source 1..183: mol_type = other DNA
aaagtgaacggctgccggaagtacaagctgaacaagaccgagagcacccctgtggtggaagaggacgagatgcagccttacgccagctacaccgagaagaacaaccctctgtacgacaccaccaacaaagtgaaggccagcgaggccctccagagcgaggttgacacagatctgcacaccctg

SEQ ID NO: 16 - protein, 21 aa, synthetic construct
IFSGFAGLLAILLVVAVFCIL

SEQ ID NO: 17 - protein, 19 aa, synthetic construct
VAVAGCVFLLISVLLLSGL

SEQ ID NO: 18 - protein, 21 aa, synthetic construct
AALAVISFLLGLGLGVACVLA

SEQ ID NO: 19 - protein, 36 aa, synthetic construct
MEADALSPVGLGLLLLPFLVTLLAALAVRARELPVS

SEQ ID NO: 20 - protein, 27 aa, synthetic construct
FWVLVVVGGVLACYSLLVTVAFIIFWV

SEQ ID NO: 21 - protein, 21 aa, synthetic construct
IVVGVVCTLLVALLMAALYLV

SEQ ID NO: 22 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 23 - skipped

SEQ ID NO: 24 - protein, 6 aa, synthetic construct
GGSGGS

SEQ ID NO: 25 - protein, 9 aa, synthetic construct
GGSGGSGGS

SEQ ID NO: 26 - protein, 12 aa, synthetic construct
GGSGGSGGSGGS

SEQ ID NO: 27 - protein, 15 aa, synthetic construct
GGSGGSGGSGGSGGS

SEQ ID NO: 28 - protein, 4 aa, synthetic construct
GGGS

SEQ ID NO: 29 - protein, 8 aa, synthetic construct
GGGSGGGS

SEQ ID NO: 30 - protein, 12 aa, synthetic construct
GGGSGGGSGGGS

SEQ ID NO: 31 - protein, 16 aa, synthetic construct
GGGSGGGSGGGSGGGS

SEQ ID NO: 32 - protein, 20 aa, synthetic construct
GGGSGGGSGGGSGGGSGGGS

SEQ ID NO: 33 - protein, 5 aa, synthetic construct
GGGGS

SEQ ID NO: 34 - protein, 10 aa, synthetic construct
GGGGSGGGGS

SEQ ID NO: 35 - protein, 15 aa, synthetic construct
GGGGSGGGGSGGGGS

SEQ ID NO: 36 - protein, 20 aa, synthetic construct
GGGGSGGGGSGGGGSGGGGS

SEQ ID NO: 37 - protein, 25 aa, synthetic construct
GGGGSGGGGSGGGGSGGGGSGGGGS

SEQ ID NO: 38 - DNA, 45 bp, synthetic construct
  source 1..45: mol_type = other DNA
ggaggcggaggatctggtggcggaggaagtggcggaggcggttct

SEQ ID NO: 39 - protein, 42 aa, synthetic construct
AAAIEVMYPPPYLDNEKSNGTIIHVKGKHLCPSPLFPGPSKP

SEQ ID NO: 40 - protein, 12 aa, synthetic construct
ESKYGPPCPSCP

SEQ ID NO: 41 - protein, 12 aa, synthetic construct
ESKYGPPAPSAP

SEQ ID NO: 42 - protein, 12 aa, synthetic construct
ESKYGPPCPPCP

SEQ ID NO: 43 - protein, 12 aa, synthetic construct
EPKSCDKTHTCP

SEQ ID NO: 44 - protein, 86 aa, synthetic construct
AAAFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 45 - protein, 45 aa, synthetic construct
TTTPAPRPPTPAPTIALQPLSLRPEACRPAAGGAVHTRGLDFACD

SEQ ID NO: 46 - protein, 132 aa, synthetic construct
ACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEADAEC

SEQ ID NO: 47 - protein, 34 aa, synthetic construct
ACPTGLYTHSGECCKACNLGEGVAQPCGANQTVC

SEQ ID NO: 48 - protein, 20 aa, synthetic construct
AVGQDTQEVIVVPHSLPFKV

SEQ ID NO: 49 - DNA, 126 bp, synthetic construct
  source 1..126: mol_type = other DNA
gcagcagctatcgaggtgatgtatcctccgccctacctggataatgaaaagagtaatgggactatcattcatgtaaaagggaagcatctttgtccttctccccttttccccggtccgtctaaacct

SEQ ID NO: 50 - DNA, 36 bp, synthetic construct
  source 1..36: mol_type = other DNA
gaaagcaagtacggtccaccttgccctagctgtccg

SEQ ID NO: 51 - DNA, 36 bp, synthetic construct
  source 1..36: mol_type = other DNA
gaatccaagtacggccccccagcgcctagtgcccca

SEQ ID NO: 52 - DNA, 36 bp, synthetic construct
  source 1..36: mol_type = other DNA
gaatctaaatatggcccgccatgcccgccttgccca

SEQ ID NO: 53 - DNA, 36 bp, synthetic construct
  source 1..36: mol_type = other DNA
gaaccgaagtcttgtgataaaactcatacgtgcccg

SEQ ID NO: 54 - DNA, 258 bp, synthetic construct
  source 1..258: mol_type = other DNA
gctgctgctttcgtacccgtgttcctccctgctaagcctacgactacccccgcaccgagaccacccacgccagcacccacgattgctagccagccccttagtttgcgaccagaagcttgtcggcctgctgctggtggcgcggtacatacccgcggccttgattttgcttgcgatatatatatctgggcgcctctggccggaacatgcggggtcctcctcctttctctggttattactctctactgtaatcacaggaat

SEQ ID NO: 55 - DNA, 396 bp, synthetic construct
  source 1..396: mol_type = other DNA
gcctgcccgaccgggctctacactcatagcggggaatgttgtaaggcatgtaacttgggtgagggcgtcgcacagccctgcggagctaaccaaacagtgtgcgaaccctgcctcgatagtgtgacgttctctgatgttgtatcagctacagagccttgcaaaccatgtactgagtgcgttggacttcagtcaatgagcgctccatgtgtggaggcagatgatgcggtctgtcgatgtgcttacggatactaccaagacgagacaacagggcggtgcgaggcctgtagagtttgtgaggcgggctccgggctggtgttttcatgtcaagacaagcaaaatacggtctgtgaagagtgccctgatggcacctactcagacgaagcagatgcagaatgc

SEQ ID NO: 56 - DNA, 102 bp, synthetic construct
  source 1..102: mol_type = other DNA
gcctgccctacaggactctacacgcatagcggtgagtgttgtaaagcatgcaacctcggggaaggtgtagcccagccatgcggggctaaccaaaccgtttgc

SEQ ID NO: 57 - DNA, 60 bp, synthetic construct
  source 1..60: mol_type = other DNA
gctgtgggccaggacacgcaggaggtcatcgtggtgccacactccttgccctttaaggtg

SEQ ID NO: 58 - protein, 588 aa, synthetic construct
METDTLLLWVLLLWVPGSTGAGGSDYKDDDDKGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITTTTPAPRPPTPAPTIALQPLSLRPEACRPAAGGAVHTRGLDFACDFWVLVVVGGVLACYSLLVTVAFIIFWVPAPAERPLPNPEGLDSDFLAVLSDYPSPDISPPIFRRGEKLRVISDEGGWWKAISLSTGRESYIPGICVARVYHGWLFEGLGRDKAEELLQLPDTKVGSFMIRESETKKGFYSLSVRHRQVKHYRIFRLPNNWYYISPRLTFQCLEDLVNHYSEVADGLCCVLTTPCLTQSTAAPAVRASSSPVTLRQKTVDWRRVSRLQEDPEGTENPLGVDESLFSYGLRESIASYLSLTSEDNTSF

SEQ ID NO: 59 - protein, 504 aa, synthetic construct
MALPVTALLLPLALLLHAARPQVQLVQSGAEVKKPGASVKVSCKASGYTFTNYWMHWVRQAPGQGLEWMGFITPTTGYPEYNQKFKDRVTMTADKSTSTAYMELSSLRSEDTAVYYCARRKVGKGVYYALDYWGQGTTVTVSSGGGGSGGGGSGGGGSDIQMTQSPSSLSASVGDRVTITCRASGNIHNYLAWYQQKPGKVPKLLIYNTKTLADGVPSRFSGSGSGTDYTLTISSLQPEDVATYYCQHFWSSPWTFGGGTKVEIKEQKLISEEDLNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 84 aa, synthetic construct
HRWCSNKKNAAVMDQESAGNRTANSEDSDEQDPQEVTYTQLNHCVFTQRKITRPSQRPKTPPTDIIVYTELPNAESRSKVVSCP

SEQ ID NO: 61 - protein, 41 aa, synthetic construct
MTDSVIYSMLELPTATQAQNDYGPQQKSSSSRPSCSCLGSG

SEQ ID NO: 62 - protein, 101 aa, synthetic construct
HRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 63 - protein, 116 aa, synthetic construct
LRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 64 - protein, 167 aa, synthetic construct
RRQRHSKHRTSDQRKTDFQRPAGAAETEPKDRGLLRRSSPAADVQEENLYAAVKDTQSEDRVELDSQSPHDEDPQAVTYAPVKHSSPRREMASPPSSLSGEFLDTKDRQVEEDRQMDTEAAASEASQDVTYAQLHSLTLRRKATEPPPSQEGEPPAEPSIYATLAIH

SEQ ID NO: 65 - protein, 168 aa, synthetic construct
QHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYATLAIH

SEQ ID NO: 66 - protein, 141 aa, synthetic construct
KLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 67 - protein, 126 aa, synthetic construct
KILPKRRTQTETPRPRFSRHSTILDYINVVPTAGPLAQKRNQKATPNSPRTPLPPGAPSPESKKNQKKQYQLPSFPEPKSSTQAPESQESQEELHYATLNFPGVRPRPEARMPKGTQADYAEVKFQ

SEQ ID NO: 68 - DNA, 252 bp, synthetic construct
  source 1..252: mol_type = other DNA
caccggtggtgcagcaacaagaaaaacgccgccgtgatggaccaagagagcgccggaaatagaaccgccaacagcgaggatagcgacgagcaggaccctcaagaagtgacctacacacagctgaaccactgcgtgttcacccagcggaagatcaccagacctagccagcggcctaagacaccacctaccgacatcatcgtgtacaccgagctgcccaacgccgagagcagatccaaggtcgtgtcctgtcct

SEQ ID NO: 69 - DNA, 123 bp, synthetic construct
  source 1..123: mol_type = other DNA
atgaccgacagcgtgatctacagcatgctcgagctgcctacagccacacaggcccagaatgattacggccctcagcagaagtccagctccagcagacctagctgtagctgtcttggctccggc

SEQ ID NO: 70 - DNA, 303 bp, synthetic construct
  source 1..303: mol_type = other DNA
caccggcagaaccagatcaagcagggccctcctagaagcaaggacgaggaacagaagcctcagcagaggcctgatctggccgtggacgtgctggaaagaacagccgataaggccaccgtgaacggcctgcctgagaaggacagagagacagacacatctgccctggccgctggcagctcccaagaagtgacatacgcccagctggaccactgggccctgacacagagaactgccagagctgtgtcccctcagagcaccaaacctatggccgagagcatcacctatgccgccgtggccagacat

SEQ ID NO: 71 - DNA, 348 bp, synthetic construct
  source 1..348: mol_type = other DNA
ctgcggcacagaaggcagggcaagcactggacaagcacccagagaaaggccgattttcagcaccctgctggcgccgttggacctgagcctacagatagaggactgcagtggcggtctagccctgctgccgatgctcaagaggaaaacctgtacgccgccgtgaaggacacccaacctgaagatggcgtggaaatggacaccagagccgccgcatctgaagcccctcaggatgtgacatacgcccagctgcatagcctgacactgaggcggaaagccacagagcctccacctagccaagagagagagcctcctgccgagcctagcatctatgccacactggccattcac

SEQ ID NO: 72 - DNA, 501 bp, synthetic construct
  source 1..501: mol_type = other DNA
cggagacagagacacagcaagcacagaaccagcgaccagagaaagaccgacttccagaggcctgctggcgccgctgagacagagcctaaagatagaggactgctgcggagaagcagccctgctgccgatgtgcaagaggaaaatctgtacgccgccgtgaaggacacccagagcgaggatagagtggaactggacagccagtctcctcacgacgaagatcctcaggccgtgacatacgcccctgtgaagcacagcagccctcggagagaaatggcctctccaccttctagcctgagcggcgagttcctggacaccaaggacagacaggtggaagaggaccggcagatggatacagaagctgccgcctctgaagccagccaggatgtgacatatgcccagctgcatagcctgacactgcggcggaaagctacagagcctcctccatctcaagagggcgagcctccagccgagcctagcatctatgccacactggccattcac

SEQ ID NO: 73 - DNA, 504 bp, synthetic construct
  source 1..504: mol_type = other DNA
cagcattggagacagggaaagcacagaacactggcccagcggcaggccgatttccaaagacctccaggtgccgccgaacctgagcctaaagatggtggcctgcagcggagatcttctcctgctgccgatgtgcagggcgagaatttttgtgccgccgtgaagaacacccagcctgaggatggcgtggaaatggacaccagacagagccctcacgacgaggatccacaggccgtgacatacgccaaagtgaagcacagccggcctcggagagaaatggctagccctccaagtcctctgagcggcgagttcctggacaccaaagacagacaggccgaagaggaccggcagatggatacagaagctgccgcctctgaagcccctcaggatgtgacatatgcccagctgcatagcttcaccctgcggcagaaagccacagagcctccaccttctcaagagggcgcttctccagccgagccatctgtgtatgccacactggccattcac

SEQ ID NO: 74 - DNA, 423 bp, synthetic construct
  source 1..423: mol_type = other DNA
aagctgcagcggagatggaagagaacacagagccagcagggcctgcaagagaatagcagcggccagagcttcttcgtgcggaacaagaaagtgcggagagcccctctgtctgagggacctcatagcctgggctgttacaaccccatgatggaagatggcatcagctacaccacactgcggttccccgagatgaacatccctagaacaggcgacgccgagagcagcgaaatgcaaagacctcctcctgactgcgacgacaccgtgacatatagcgccctgcacaagagacaagtgggcgactacgagaacgtgatccctgacttccctgaggacgagggcatccactacagcgagctgatccagtttggcgtgggcgaaagaccccaggctcaagagaatgtggactacgtgatcctgaagcac

SEQ ID NO: 75 - DNA, 378 bp, synthetic construct
  source 1..378: mol_type = other DNA
aagatcctgcctaagcggcgcacccagaccgagactcctagacctagattcagccggcacagcaccatcctggactacatcaacgtggtgcctaccgccggaccactggctcagaagagaaaccagaaggccacacctaacagccccagaacacctcttccacctggcgcaccttctccagagagcaagaagaaccagaagaagcagtaccagctgcctagcttccccgagcctaagagcagtacacaggcccctgagagccaagagtcccaagaggaactgcactacgccacactgaacttccccggcgtcagacctagacctgaggccagaatgcctaagggcacccaggccgattacgccgaagtgaagtttcaa

SEQ ID NO: 76 - protein, 19 aa, synthetic construct
ILIGTSVVIILFILLFFLL

SEQ ID NO: 77 - protein, 21 aa, synthetic construct
VIGILVAVILLLLLLLLLFLI

SEQ ID NO: 78 - protein, 21 aa, synthetic construct
VAIALGLLTAVLLSVLLYQWI

SEQ ID NO: 79 - protein, 21 aa, synthetic construct
ILIGVSVVFLFCLLLLVLFCL

SEQ ID NO: 80 - protein, 21 aa, synthetic construct
VIGILVAVVLLLLLLLLLFLI

SEQ ID NO: 81 - protein, 21 aa, synthetic construct
VLIGVSVAFVLLLFLLLFLLL

SEQ ID NO: 82 - protein, 21 aa, synthetic construct
VLIGVLVVSILLLSLLLFLLL

SEQ ID NO: 83 - protein, 19 aa, synthetic construct
VAVGLGSCLAILILAICGL

SEQ ID NO: 84 - protein, 21 aa, synthetic construct
GAFLGIGITALLFLCLALIIM

SEQ ID NO: 85 - DNA, 57 bp, synthetic construct
  source 1..57: mol_type = other DNA
atcctgatcggcaccagcgtggtcatcatcctgtttatcctgctgttcttcctgctg

SEQ ID NO: 86 - DNA, 63 bp, synthetic construct
  source 1..63: mol_type = other DNA
gtgatcggcattctggtggccgtgattctgctgctcctgctgttgctgctgctgttcctgatc

SEQ ID NO: 87 - DNA, 63 bp, synthetic construct
  source 1..63: mol_type = other DNA
gtggccattgctctgggactgcttacagccgtgctgctgagtgtgctgctgtaccagtggatc

SEQ ID NO: 88 - DNA, 63 bp, synthetic construct
  source 1..63: mol_type = other DNA
atcctgatcggagtgtccgtggtgtttctgttctgcctgctcctgctggtgctgttctgtctg

SEQ ID NO: 89 - DNA, 63 bp, synthetic construct
  source 1..63: mol_type = other DNA
gtgatcggaattctggtggccgtggtgctgctcctgctgcttctccttctgctgttcctgatc

SEQ ID NO: 90 - DNA, 63 bp, synthetic construct
  source 1..63: mol_type = other DNA
gtgctgatcggagtgtctgtggctttcgtgctgctcctgttcctcctgctgttcctgctcctg

SEQ ID NO: 91 - DNA, 63 bp, synthetic construct
  source 1..63: mol_type = other DNA
gtgctgattggcgtgctggtggtgtctatcctgctcctgtcactgctgctgtttctgctgctc

SEQ ID NO: 92 - DNA, 57 bp, synthetic construct
  source 1..57: mol_type = other DNA
gtggccgttggcctgggatcttgtctggccattctgatcctggccatctgcggcctg

SEQ ID NO: 93 - DNA, 63 bp, synthetic construct
  source 1..63: mol_type = other DNA
ggcgcctttctcggcatcggaattacagccctgctgttcctgtgcctggctctgatcatcatg

SEQ ID NO: 94 - protein, 75 aa, synthetic construct
TTTPAPRPPTPAPTIALQPLSLRPEACRPAAGGAVHTRGLDFACDQTTPGERSSLPAFYPGTSGSCSGCGSLSLP

SEQ ID NO: 95 - protein, 463 aa, synthetic construct
MALPVTALLLPLALLLHAARPKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDILIGTSVVIILFILLFFLLHRWCSNKKNAAVMDQESAGNRTANSEDSDEQDPQEVTYTQLNHCVFTQRKITRPSQRPKTPPTDIIVYTELPNAESRSKVVSCP

SEQ ID NO: 96 - protein, 422 aa, synthetic construct
MALPVTALLLPLALLLHAARPKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDVIGILVAVILLLLLLLLLFLIMTDSVIYSMLELPTATQAQNDYGPQQKSSSSRPSCSCLGSG

SEQ ID NO: 97 - protein, 422 aa, synthetic construct
MALPVTALLLPLALLLHAARPKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDVAIALGLLTAVLLSVLLYQWIMTDSVIYSMLELPTATQAQNDYGPQQKSSSSRPSCSCLGSG

SEQ ID NO: 98 - protein, 482 aa, synthetic construct
MALPVTALLLPLALLLHAARPKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 99 - protein, 497 aa, synthetic construct
MALPVTALLLPLALLLHAARPKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 100 - protein, 548 aa, synthetic construct
MALPVTALLLPLALLLHAARPKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDVLIGVSVAFVLLLFLLLFLLLRRQRHSKHRTSDQRKTDFQRPAGAAETEPKDRGLLRRSSPAADVQEENLYAAVKDTQSEDRVELDSQSPHDEDPQAVTYAPVKHSSPRREMASPPSSLSGEFLDTKDRQVEEDRQMDTEAAASEASQDVTYAQLHSLTLRRKATEPPPSQEGEPPAEPSIYATLAIH

SEQ ID NO: 101 - protein, 549 aa, synthetic construct
MALPVTALLLPLALLLHAARPKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDVLIGVLVVSILLLSLLLFLLLQHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYATLAIH

SEQ ID NO: 102 - protein, 520 aa, synthetic construct
MALPVTALLLPLALLLHAARPKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 103 - protein, 507 aa, synthetic construct
MALPVTALLLPLALLLHAARPKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDGAFLGIGITALLFLCLALIIMKILPKRRTQTETPRPRFSRHSTILDYINVVPTAGPLAQKRNQKATPNSPRTPLPPGAPSPESKKNQKKQYQLPSFPEPKSSTQAPESQESQEELHYATLNFPGVRPRPEARMPKGTQADYAEVKFQ

SEQ ID NO: 104 - protein, 426 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDILIGTSVVIILFILLFFLLHRWCSNKKNAAVMDQESAGNRTANSEDSDEQDPQEVTYTQLNHCVFTQRKITRPSQRPKTPPTDIIVYTELPNAESRSKVVSCP

SEQ ID NO: 105 - protein, 385 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDVIGILVAVILLLLLLLLLFLIMTDSVIYSMLELPTATQAQNDYGPQQKSSSSRPSCSCLGSG

SEQ ID NO: 106 - protein, 385 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDVAIALGLLTAVLLSVLLYQWIMTDSVIYSMLELPTATQAQNDYGPQQKSSSSRPSCSCLGSG

SEQ ID NO: 107 - protein, 445 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 108 - protein, 460 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 109 - protein, 511 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDVLIGVSVAFVLLLFLLLFLLLRRQRHSKHRTSDQRKTDFQRPAGAAETEPKDRGLLRRSSPAADVQEENLYAAVKDTQSEDRVELDSQSPHDEDPQAVTYAPVKHSSPRREMASPPSSLSGEFLDTKDRQVEEDRQMDTEAAASEASQDVTYAQLHSLTLRRKATEPPPSQEGEPPAEPSIYATLAIH

SEQ ID NO: 110 - protein, 512 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDVLIGVLVVSILLLSLLLFLLLQHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYATLAIH

SEQ ID NO: 111 - protein, 483 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 112 - protein, 470 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDGAFLGIGITALLFLCLALIIMKILPKRRTQTETPRPRFSRHSTILDYINVVPTAGPLAQKRNQKATPNSPRTPLPPGAPSPESKKNQKKQYQLPSFPEPKSSTQAPESQESQEELHYATLNFPGVRPRPEARMPKGTQADYAEVKFQ

SEQ ID NO: 113 - DNA, 717 bp, synthetic construct
  source 1..717: mol_type = other DNA
caggtgcagctgaaagagtctggacctggactggtgcagcccagccaaacactgagcctgacctgtaccgtgtccggcttcagcctgagcagatacgacatgcactgggtccgacagcctccaggacaaggcttggaatggatgggcgtgatctggggcaacggcaacacacactatcacagcgccctgaagtcccggctgagcatcagcagagataccagcaagagccaggtgttcctgaagatgaacagcctgcagaccgaggacaccgccatctatttctgcaccctgcggatcaaggattggggccctggcacaatggtcaccgtttctagcggaggcggaggatctggtggcggaggaagtggcggaggcggttctgatatcgtgatgacccagacacctcctagcctgtctgtggctctgggccagtctgtgtccatcagctgcaagagcagccagagcctggtggcctccgacgagaacacctacctgaattggctgctgcagagccccggcagaagccccaagagactgatctaccaggtgtccaagctggacagcggcgtgcccgatagattttctggcagcggcagcgagaaggacttcaccctgaagatctccagagtggaagccgaggacctgggcgtgtactactgtctgcaaggcatccatctgccttggacctttggaggcggcacaaagctggaactgaaa

SEQ ID NO: 114 - protein, 244 aa, synthetic construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIK

SEQ ID NO: 115 - protein, 504 aa, synthetic construct
METDTLLLWVLLLWVPGSTGAGGSDYKDDDDKGGSEVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIKTTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - DNA, 1524 bp, synthetic construct
  source 1..1524: mol_type = other DNA
gccgccaccatggaaaccgacacactgctgctgtgggtgctgcttctttgggtgcccggatctacaggtgccggcggaagcgactacaaggacgacgatgacaaaggcggcagcgaggttcaactggtacaaagcggagccgaggtaaagaaaccagggagtagcgtcaaagtgtcctgcaaagcctcaggcggcacattcagtagctatgctatttcatgggtacgccaagcaccaggacaggggctggagtggatgggcgggattatccccatcttcggtacggcaaactatgcacaaaagttccagggacgagtcaccatcacggctgataagtccacctccaccgcctatatggagctgagttcccttcggagcgaggatactgctgtgtattattgtgccacgttcgcactgttcggttttcgggagcaggcgtttgatatttggggacaaggcacaacggtcacggtcagttcaggcggagggggatcagggggtggggggtcaggtggcggtggaagtgacattcagatgacccagagtccctcttcattgagtgcgagcgtcggtgatcgggttacgataacctgtagggcctcccaaagtatatcatcatatttgaactggtaccaacagaaacctgggaaagcgccgaagctccttatctatgctgccagctctttgcaaagcggtgtgccctcacggttctccggtagtgggtccgggaccgacttcactttgaccatcagcagccttcagccagaggatcttgccacttattactgccagcaatcttatagcacaccgtttacattcggtccaggcacaaaggtagacattaagaccaccacaccagctcctagacctccaactcctgctcctacaatcgccctgcagccactgagtctgaggccagaggcttgtagacctgctgcaggcggagccgtgcatacaagaggactggatttcgcctgcgacttctgggtgctcgtggttgttggcggagtgctggcctgttacagcctgctggttaccgtggccttcatcatcttttgggtccgaagcaagcggagccggctgctgcacagcgattacatgaacatgacccctcggaggcccggacctaccagaaagcactaccagccttacgctcctcctagagatttcgccgcctaccggtccagagtgaagttcagcagatccgccgatgctcccgcctataagcagggccagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaagataa